(19) United States
(12) Patent Application Publication
(10) Pub. No.: US 2019/0264285 A1
(43) Pub. Date: Aug. 29, 2019

(54) POLYMORPHISMS FOR PREDICTING TREATMENT RESPONSE TO ANTIPSYCHOTIC DRUGS AND IDENFYING NEW DRUG TARGETS

(71) Applicant: Northwestern University, Evanston, IL (US)

(72) Inventors: Jiang Li, Chicago, IL (US); Herbert Y. Meltzer, Chicago, IL (US)

(73) Assignee: Northwestern University, Evanston, IL (US)

(21) Appl. No.: 16/284,423

(22) Filed: Feb. 25, 2019

Related U.S. Application Data
(60) Provisional application No. 62/634,390, filed on Feb. 23, 2018.

(51) Int. Cl. C12Q 1/6883; C12Q 1/6827; C12Q 1/682; G16H 50/20; G16H 50/30; G16B 20/20; G16B 20/10; G01N 33/68

(52) U.S. Cl. CPC C12Q 1/6883; C12Q 1/6827; C12Q 1/682; G16H 50/20; G01N 2800/302; G16B 20/20; G16B 20/10; G01N 33/6893; G16H 50/30

(57) ABSTRACT

Disclosed are methods, kits, and devices for diagnosing and treating psychiatric disorders and the symptoms thereof. The methods, kits, and devices relate to identifying genetic markers that may be utilized to diagnose and/or prognose a subject and treat the diagnosed and/or prognosed subject by administering a drug the subject based on the genetic marker having been identified. Genetic markers identified in the methods may include a polymorphism in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions. The disclosed methods, kits, and devices have implications for developing new antipsychotic drugs that target the activity of proteins associated with the identified genetic markers and for diagnosing/prognosing a response to an antipsychotic drug based on the genetic markers.

### CROSS-REFERENCE TO RELATED PATENT APPLICATIONS

The present application claims the benefit of priority under 35 U.S.C. § 119(e) to U.S. Provisional Patent Application No. 62/634,390, filed on Feb. 23, 2018, the content of which is incorporated herein by reference in its entirety.

### BACKGROUND

The invention relates to methods for diagnosing and treating psychiatric disorders. In particular, the intention relates to methods for identifying genetic markers that are associated with treatment response for psychiatric disorders in a subject and administering drugs to the subject based on identifying the genetic markers. The genetic markers may include polymorphisms such as single nucleotide polymorphisms (SNPs) and the drugs may include typical and atypical antipsychotic drugs (APDs).

Antipsychotic drugs (APDs) are more effective to treat positive (psychotic) than negative symptoms or cognitive impairment in schizophrenia (SCZ). Psychotic symptoms respond to APDs in approximately 70% of subjects with SCZ who may be classified as non-treatment resistant SCZ (non-TRS). The other ˜30% have moderate-severe positive symptoms after two or more trials with APDs and are referred to as treatment resistant SCZ (TRS) (Meltzer, 2012). Individual genetic, epigenetic, adherence, and other factors which affect drug absorption, metabolism, and interaction with various concomitant treatments account for the large variation in extent and time course of clinical response to APDs. Identifying multiple genetic and other biomarkers which contribute to these differences would facilitate optimal drug choice and might also lead to novel targets for APDs.

Here, the inventors disclose GWAS which analyzed data from two clinical trials of an atypical APD in acutely psychotic SCZ subjects with European or African Ancestry (AA) (Meltzer et al., 2011b; Nasrallah et al., 2013b). The inventors identified SNPs and pathways associated with change in PANSS total (ΔPANSS-T) and PANSS subscales which predicted efficacy and identified possible novel drug targets.

### SUMMARY

Disclosed are methods, kits, and devices for diagnosing and treating psychiatric disorders and the symptoms thereof. The methods, kits, and devices relate to identifying genetic markers that may be utilized to diagnose and/or prognose a subject and treat the diagnosed and/or prognosed subject by administering a drug the subject based on the genetic marker having been identified. In some embodiments, genetic markers identified in the methods may include polymorphic alleles of polymorphisms in genes encoding proteins associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions. Based on the polymorphic alleles being identified in the subject, the subject may be identified as having responsiveness to an antipsychotic drug (APD), such as a typical APD or an atypical APD. As such, the subject may be treated by administering the APD to treat the psychiatric disorder and/or the symptoms thereof after the polymorphic alleles has been identified.

In some embodiments, the disclosed methods are related to methods for treating a psychiatric disorder or the symptoms thereof in a subject. The disclosed methods may comprise the following steps: (a) determining whether the subject has a polymorphic alleles, or receiving the results of test indicating that a subject has a polymorphic alleles; and (b) administering an antipsychotic drug (APD) if the subject has the polymorphic alleles. In the disclosed methods, the psychiatric disorder may include, but is not limited to schizophrenia (e.g., schizophrenia characterized by positive symptoms, negative symptoms, and/or cognitive symptoms), bipolar disorder, and psychiatric depression with psychotic features. In some embodiments, suitable polymorphisms detected in the methods or indicated in the test results utilized in the methods may include, but are not limited to a polymorphism in one or more genes selected from the group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

In some embodiments, the polymorphism is selected from one or more of the following: rs17596267, rs4854914, rs11223651, rs732381, rs133051, rs3759111, rs1222385, rs10897829, rs1402992, rs2237326, rsrs11120818, rs10484399, rs13207082, rs13198716, rs9467714, rs7749823, rs6940698, rs12058768, rs7551092, rs6720194, rs12479448, rs7581086, rs11898081, rs10169158, rs11919644, rs11713798, rs7615029, rs6787048, rs3904759, rs7666965, rs12513242, rs2169595, rs2575635, rs2077246, rs13106118, rs200031, rs4495147, rs17311549, rs17139218, rs17145587, rs3849067, rs2085575, rs6923772, rs11154344, rs10440955, rs2906199, rs1537593, rs17153105, rs2729547, rs13278546, rs17595134, rs6999334, rs9644441, rs1500318, rs10959577, rs7899847, rs17229781, rs12766218, rs12768287, rs1416924, rs11596919, rs7101596, rs11219139, rs11062907, rs7954760, rs9579835, rs12859383, rs7330675, rs12881028, rs16952671, rs11634382, rs16940273, rs16940448, rs7166706, rs7166722, rs294267, rs30012, rs4889551, rs16955317, rs225284, rs225255, rs7208758, rs1893243, rs10502610, rs585811, rs2819956, rs234324, rs2767607, rs17755028, rs17755054, rs6019817, rs2830909, rs742002, rs2160409, rs1531802, rs4027073, rs10069504, rs4865610, rs10474643, rs4712608, rs1005886, rs7743963, rs12208773, rs1168055, rs12719654, rs10091071, rs1495074, rs16879886, rs2512434, rs10505506, rs7010421, rs7017126, rs2468720, rs2169623, rs2447553, rs2093483, rs16909902, rs1805155, rs2282040, rs2282041, rs10512249, rs16909927, rs10512247, rs7042032, rs12255425, rs9971172, rs11187065, rs7919740, rs12241284, rs11192261, rs9783155, rs1507642, rs10768525, rs10837219, rs10837221, rs11035428, rs11035429, rs1676667, rs1702585, rs1676664, rs12364216, rs1940751, rs11600281, rs11110065, rs10860532, rs1387082, rs11110270, rs11110297, rs3887427, rs3884623, rs292462, rs4901072, rs8010726, rs12917416, rs2202979, rs28405182, rs9933246, rs9935875, rs9935962, rs9924951, rs9936248, rs10459843, rs11649628, rs726476, rs8048158, rs11077179, rs10468333, rs8045750, rs8057315, rs11641748, rs17674225, rs2965886, rs8048077, rs7195330, rs12597561, rs12950365, rs10512467, rs11653010, rs9960395, rs8093330, rs8110501, rs8131774, rs9980586, rs1699695, rs2828827, rs2828835, rs2832478, rs12835711, rs11120818, rs7101596, rs9644441, rs11596919, rs7551092, rs7239345, rs7166706, rs7166722, rs17595134, rs11588846, rs1816382, rs225255, rs2819166, rs11919644, rs9376913, rs16838, rs10440955, rs153479, rs11898081, rs12768287, rs1416924, rs9557996, rs10502610, rs1893243, rs41446249, rs2945908, rs2077246, rs6814341, rs7827390, rs17106441, rs10749378, rs11713798, rs6787048, rs2215381, rs1356374, rs12510684, rs7330675, rs7208758, rs7644745, rs12766218, rs11133186, rs2430807, rs9824811, rs321601, rs225284, rs13271251, rs6813301, rs1407066, rs2319068, rs503562, rs4748050, rs4750396, rs1500318, rs250585, rs9579835, rs403904, rs6500606, rs7243239, rs2575635, rs1980945, rs1676664, rs1702585, rs10837219, rs10837221, rs11035429, rs10512247, rs2169623, rs10512249, rs16909902, rs2282040, rs1676667, rs2447553, rs11035428, rs2282041, rs292462, rs1805155, rs292459, rs16975933, rs9524948, rs4601698, rs2468717, rs8110501, rs11110297, rs17118088, rs2160409, rs10768525, rs1507642, rs12126638, rs11192261, rs9783155, rs6717347, rs7204304, rs12929401, rs8010726, rs11591402, rs12364216, rs2391376, rs11110270, rs3887427, rs2003990, rs2745822, rs7313402, rs4076584, rs9341130, rs11808980, rs12241284, rs7919740, rs899073, rs17655606, rs3744635, rs2093483, rs2325882, rs402914, rs7946725, rs1908159, rs10171741, rs11649628, rs9935875, rs9935962, rs7302443, rs1206069, rs3884623, rs1032932, rs4964658, rs678697, rs4140729, rs6742598, rs3847178, rs1168055, rs10204599, rs2512434, rs4772812, rs2290273, rs11856774, rs16969710, rs11712608, rs16975932, rs10143206, rs7144971, rs6700661, rs10474643, rs12233091, rs8045750, rs17152573, rs17674225, rs10860532, rs11110065, rs2201331, rs976368, rs2832478, rs7973562, rs1387082, rs12447542, rs10500355, rs1057521725, rs1064794750, rs11643447, rs11645781, rs11866781, rs12444931, rs12446308, rs12921846, rs12926282, rs1478693, rs17139207, rs17139244, rs17648524, rs1906060, rs3785234, rs4124065, rs4146812, rs4786816, rs4787008, rs6500742, rs6500744, rs6500818, rs6500882, rs6500963, rs716508, rs7191721, rs7403856, rs7498702, rs870288, rs889699, rs9302841, rs9924951, rs1478697, rs4736253, rs10180106, rs511841, rs10895475, rs7017126, rs3857923, rs13270196, rs964441, rs13394481, rs1541947, rs16879886, rs11922361, rs2093483, rs524045, rs4733373, rs16879886, rs2239037, rs2007044, rs9636107, rs971215, and any combination thereof.

Suitable polymorphisms may be polymorphisms associated with the RBFOX1 (A2BP1) gene. In some embodiments, the polymorphism is selected from one or more of the following: rs17674225 (e.g., where the allele is G/T), rs8057315 (e.g., where the allele is C/A/G/T), rs726476 (e.g., where the allele is G/A/C/T), rs8045750 (e.g., where the allele is G/A), rs9924951 (e.g., where the allele is G/A), rs10468333 (e.g., where the allele is C/G/T), rs9933246 (e.g., where the allele is G/C/T), rs8048158 (e.g., where the allele is C/G), rs11077179 (e.g., where the allele is T/C), rs9936248 (e.g., where the allele is C/A), rs11641748 (e.g., where the allele is G/A), rs10459843 (e.g., where the allele is G/A/C), rs9935875 (e.g., where the allele is G/A/C), rs9935962 (e.g., where the allele is C/A), rs11649628 (e.g., where the allele is C/T), rs28405182 (e.g., where the allele is C/A/G/T), rs8048519 (e.g., where the allele is A/G), rs2159535 (e.g., where the allele is G/C), rs11077183 (e.g., where the allele is C/A), rs11077184 (e.g., where the allele is A/C/G), rs7198769 (e.g., where the allele is G/A/T), rs4786173 (e.g., where the allele is G/A), rs4141146 (e.g., where the allele is G/A), rs9935875 (e.g., where the allele is G/A), rs9935962 (e.g., where the allele is C/A), rs8057315 (e.g., where the allele is C/A/G/T), rs8045750 (e.g., where the allele is A/G), rs17674225 (e.g., where the allele is C/G/T), rs12447542 (e.g., where the allele is A/G), rs10500355 (e.g., where the allele is A/T), rs1057521725 (e.g., where the allele is A/G), rs1064794750 (e.g., where the allele is G/C), rs11643447 (e.g., where the allele is A/T), rs11645781 (e.g., where the allele is A/G), rs11866781 (e.g., where the allele is C/T), rs12444931 (e.g., where the allele is A/G), rs12446308 (e.g., where the allele is A/G), rs12921846 (e.g., where the allele is A/T), rs12926282 (e.g., where the allele is A/C), rs1478693 (e.g., where the allele is A//C), rs17139207 (e.g., where the allele is A/G), rs17139244 (e.g., where the allele is A/G), rs17648524 (e.g., where the allele is C/G), rs1906060 (e.g., where the allele is C/T), rs3785234 (e.g., where the allele is C/T), rs4124065 (e.g., where the allele is G/T), rs4146812 (e.g., where the allele is C/T), rs4786816 (e.g., where the allele is A/G), rs4787008 (e.g., where the allele is A/G), rs6500742 (e.g., where the allele is C/T), rs6500744 (e.g., where the allele is C/T), rs6500818 (e.g., where the allele is C/T), rs6500882 (e.g., where the allele is G/T), rs6500963 (e.g., where the allele is C/T), rs716508 (e.g., where the allele is C/T), rs7191721 (e.g., where the allele is A/G), rs7403856 (e.g., where the allele is A/G), rs7498702 (e.g., where the allele is C/T), rs870288 (e.g., where the allele is A/G), rs889699 (e.g., where the allele is A/G), rs9302841 (e.g., where the allele is A/T), rs9924951 (e.g., where the allele is A/G), rs1478697 (e.g., where the allele is A/G/T), and combinations thereof.

In some embodiments, the disclosed methods include administering an antipsychotic drug (APD). The APD may exhibit a number of biological activities including, but not limited to antagonism of one or more of the following sites, α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor. In some embodiments, the APD may exhibit at least partial agonism of the 5-HT_{1A} receptor. In some embodiments, the APD may exhibit negligible or no biological activity as a ligand for the H₁ receptor and/or the mACh receptor.

In some embodiments of the disclosed methods, the subject may have undergone treatment prior to the disclosed methods being performed and the subject may have been diagnosed with a treatment resistant psychiatric disorder prior to the method being performed. Accordingly, the methods contemplated herein include methods for determining treatment responsiveness and treating subjects with the appropriate APD.

In some embodiments, the presently disclosed methods relate to polymorphisms and detecting polymorphic alleles. The disclosed methods may include determining or detecting a nucleotide sequence associated with the polymorphisms in a nucleic acid sample from a subject and/or determining or detecting whether the subject comprises one or more of an A-allele, a C-allele, a G-allele, and/or a T-allele. In some embodiments, the methods including determining whether the subject has one or more polymorphic alleles by sequencing a nucleic acid sample obtained from the subject. In other embodiments, the methods may include determining whether the subject has one or more polymorphic alleles by treating a nucleic acid sample obtained from the subject with a nucleic acid probe (e.g. a probe that hybridizes specifically to a nucleic acid sequence comprising the polymorphic allele). The methods may include determining whether the subject is homozygous or heterozygous for a polymorphic allele. Further, the methods may include administering a pharmaceutical agent if the subject is found to be homozygous or heterozygous for the polymorphic allele.

The disclosed methods typically include treating a subject based on the genotype of the subject with respect to the polymorphism. For example, the methods typically include administering a pharmaceutical agent to the subject if the subject is homozygous or heterozygous for a polymorphic allele of a polymorphism.

Also disclosed herein are kits and devices for performing the disclosed methods, and systems comprising the disclosed kits and devices. For example, the disclosed kits and devices may include and/or utilize reagents for diagnosing, prognosing, and/or treating a psychiatric disease or disorder or the symptoms thereof in a subject. The presently disclosed kits and devices may include and/or utilize reagents such as: (a) reagents for detecting the genotype of a subject in regard to a polymorphism. The kits and devices may include and/or utilize reagents for amplifying and or sequencing nucleic acid comprising one or more polymorphic alleles of one or more polymorphisms and/or reagents for probing nucleic acid comprising one or more polymorphic alleles of one or more polymorphisms; and optionally (b) a pharmaceutical agent comprising an atypical drug for treating a psychiatric disease or disorder (e.g., lurasidone, ziprasidone, clozapine, olanzapine, risperidone, perphenazine, and sertindole). The reagents in the kit may include nucleic acid reagents (e.g., primers and/or probes that hybridize to a polymorphic allele and that may be utilized to amplify, sequence, and/or probe the polymorphic allele or an RNA expressed from the polymorphic allele) and non-nucleic acid reagents (e.g., polymerases and buffers). The pharmaceutical agent of the kits and devices may include a typical or atypical APD for treating the psychiatric disease or disorder formulated for administration to the subject.

Also disclosed are methods for treating psychiatric diseases or disorders in a subject in need thereof. The methods may include administering a therapeutic agent that increases expression and/or activity of RBFOX1, which may include a small molecule therapeutic agent.

Also disclosed are methods for identifying a therapeutic agent for treating psychiatric diseases or disorders. The methods may include screening a library of therapeutic agents for a therapeutic agent that increases expression and/or activity of RBFOX1, and identifying a therapeutic agent that increases expression and/or activity of RBFOX1 as the therapeutic agent for treating psychiatric diseases or disorders. The therapeutic thus identified may be formulated as a pharmaceutical composition for treating psychiatric diseases or disorders.

### BRIEF DESCRIPTION OF THE FIGURES

FIG. 1. Manhattan and QQ plots for GWAS results associated with ΔPANSS-T. 1a and 1c. Caucasians; 1b and 1d. African-American. Manhattan plot of results of GWAS in association with ΔPANSS-T. Linear regression adjusted for covariates including race (PCA1-3), gender, and lurasidone dosage. Genes annotated for the genomic loci which are associated with ΔPANSS-T with unadjusted p-value<10⁻⁵ are labelled. The unadjusted p-value is the raw p-value without correction for multiple testing. All SNPs were genotyped, not imputed. λ=1.00 for both Caucasians (1c) and African-Americans (1d). There was no evidence for systematic inflation of genome-wide test statistics, as assessed by the genomic inflation factor (λ).

FIG. 2. Forest plot of the estimated fold change (FC) of gene expression with 95% confidence interval of several genes of interest when comparing post-mortem DLPFC (BA46) of SCZ subjects and controls in two independent studies. We only listed genes annotated to the genomic loci associated with ΔPANSS-T with unadjusted p<10⁻⁴ and demonstrated significant differential gene expression (p<0.05). Linear model and empirical Bayes method (LIMMA) was applied for assessment of differential gene expression in two independent datasets (GSE21138 and GSE12649) deposited in NCBI Gene Expression Omnibus (GEO). Log FC>0 suggests decreased gene expression in the schizophrenic patients. Gene name, Study Name and probeset ID were labelled in each row.

FIG. 3. Polygenic modeling using SNPs from the PGCGWAS of schizophrenia to predict treatment response to lurasidone vs placebo in Caucasians. 3a. Prediction of treatment response to lurasidone vs placebo using the GCTA-GREML model. V(G)/V(P) % represent the estimate of the phenotypic variance in Δchange in PANSS_TOT or subscales explained by the subsets of SNPs with different levels of p-values from the PGC GWAS for SCZ. “*” represent the level of significance for this association. 4c. Polygenic modeling by Polygenic Risk Scores (PRS) calculated using SNPs and log of odds ratios derived from PGC GWAS for SCZ to predict treatment response to lurasidone. Variance explained (R²) and regression coefficient of PRS and their corresponding p-values were calculated by a linear regression model adjusted for the covariates in association with A change in PANSS_POS/NEG/TOT.

FIG. 4. Illustration of the samples and analytical pipeline. PCA yielded 368 EUR, 264 and 104 in the lurasidone- and placebo-treated groups, respectively. There were 219 AFRs, 158 and 61 in the lurasidone- and placebo groups, respectively. We compared the results from placebo groups to the results from the meta-analysis and mega-analysis of lurasidone groups to determine which genetic risk factors were associated with treatment response in the lurasidone not the placebo group. These were considered to be drug-specific.

FIG. 5. Manhattan plot and QQ plot for the summary statistics of the meta-analysis in patients with European or African Ancestry. 1A and 1B. EUR; 1C and 1D. AFR. Manhattan plots for the results of GWAS in ΔPANSS-TOT_{LOCF6WK}. Linear regression adjusted for covariates including race (PCs 1-5) and lurasidone dosage. The −log 10 (p value) of each SNP was shown as a function of genomic position on the autosomes (hg19). Genome-wide significance level was denoted (dotted line, p=5×10⁻⁸); Genes annotated for the genomic loci which were associated with ΔPANSS-TOT_{LOCF6WK} with uncorrected p-value<5×10⁻⁷ were labelled. The uncorrected p-value was the raw p-value without correction for multiple testing. All SNPs were genotyped, not imputed. The genomic inflation factor λ_{GC} for pearl 12 and pearl 3 from both Caucasians (1C) and African-Americans (1D) were listed in Table 1 with each p<1.03. There was also no evidence for systematic inflation of genome-wide statistics of meta-analyses assessed by QQplot.

FIG. 6. Meta-analysis of patients with European or African Ancestry: top variants with p value<5×10⁻⁷ with LD clumping (−clump-r² 0.5).

FIG. 7. Polygenic modeling using SNPs from the PGC GWAS of schizophrenia to predict treatment response to lurasidone in EUR. Polygenic modeling by Polygenic Risk Scores (PRS) calculated using SNPs and log of odds ratios derived from PGC GWAS for SCZ to predict treatment response to lurasidone. Regression coefficients of PRS and their corresponding p-values were calculated by a linear regression model adjusted for the dosage, study, and five PCs, and present as “Regression Coefficients/P value”. The number of SNP left after LD pruning to build the polygenic model was listed at 17 consecutive levels of significant association with SCZ.

### DETAILED DESCRIPTION

Disclosed are methods, kits, and devices for diagnosing and treating psychiatric disorders and the symptoms thereof. The methods, kits, and devices are described herein using several definitions, as set forth below and throughout the application.

As used in this specification and the claims, the singular forms “a,” “an,” and “the” include plural forms unless the context clearly dictates otherwise. For example, “a polymorphism” and “a polymorphic allele” should be interpreted to mean “one or more polymorphisms” and “one or more polymorphic alleles,” respectively, unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The presently disclosed methods, kits, and devices relate to identifying genetic markers that may be utilized to diagnose and/or prognose a subject, and optionally treat the diagnosed and/or prognosed subject by administering a drug to the subject based on the genetic marker having been identified.

As used herein, the term “subject,” which may be used interchangeably with the terms “patient” or “individual,” refers to one who receives medical care, attention or treatment and may encompass a human subject. As used herein, the term “subject” is meant to encompass a person who has a psychiatric disorder or is at risk for developing a psychiatric disorder, which includes but is not limited to schizophrenia, bipolar disorder, and psychotic depression (e.g., depression with psychotic features). For example, the term “subject” is meant to encompass a person at risk for developing schizophrenia or a person diagnosed with schizophrenia (e.g., a person who may be symptomatic for schizophrenia but who has not yet been diagnosed). As used herein, “schizophrenia” may include schizophrenia characterized by positive symptoms, negative symptoms, cognitive symptoms, or any combination thereof. The term “subject” also is meant to encompass a person at risk for developing bipolar disorder or a person diagnosed with bipolar disorder (e.g., a person who may be symptomatic for bipolar disorder but who has not yet been diagnosed). The term “subject” further is meant to encompass a person at risk for developing depression (e.g., depression with psychotic features). As such, the term “subject” further is meant to encompass a person at risk for developing depression with psychotic features or a person diagnosed with depression with psychotic features (e.g., a person who may be symptomatic for depression with psychotic features but who has not yet been diagnosed).

The disclosed methods may include: (a) detecting a polymorphic allele in a nucleic acid sample from a subject having a psychiatric disease or disorder; and (b) administering an antipsychotic drug (APD) to the subject after the polymorphic allele is detected. In some embodiments, the polymorphic allele may be detected by a step that includes amplifying at least a portion of a polymorphic allele from the nucleic acid sample and detecting the polymorphic allele in the amplified portion. In further embodiments, the polymorphic allele may be detected by a step that includes sequencing at least a portion of a polymorphic allele from the nucleic acid sample or from an amplicon obtained by amplifying at least a portion of the polymorphic allele from the nucleic acid sample. In even further embodiments, the polymorphic allele may be detected by a step that includes contacting nucleic acid comprising the polymorphic allele with a nucleic acid probe that hybridizes specifically to nucleic acid comprising the polymorphic allele.

Genetic markers identified in the methods may include a variety of polymorphisms, including polymorphisms in genes encoding proteins associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions include polymorphisms. Suitable polymorphisms for the disclosed methods are disclosed throughout this application including polymorphisms disclosed in Example 1; Example 2; Example 3; Li et al., “Genetic predictors of antipsychotic response to lurasidone identified in a genome wide association study and by schizophrenia risk genes,” Schizophr. Res., 192 (2018) 194-204, 19 Apr. 2017; and Li et al., “Identifying the genetic risk factors for treatment response to lurasidone by genome-wide association study: A meta-analysis of samples from three independent clinical trials,” Schizophr. Res. 2018 September; 198: 203-213, epub May 2, 2018; the contents of which are incorporated herein by reference in their entireties.

Exemplary polymorphisms detected in the disclosed methods may include, but are not limited to a polymorphism in a gene selected from a group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2. The disclosed methods may include determining whether a subject is homozygous or heterozygous for the polymorphism (e.g., by determining whether a nucleic acid sample from the subject is homozygous or heterozygous for the polymorphic allele). Methods, compositions, and kits for diagnosing, prognosing, and treating psychiatric disorders that include steps of detecting genetic polymorphisms are disclosed in U.S. Published Application No. 2015/0099741, the content of which is incorporated herein by reference in its entirety.

The disclosed methods may include detecting a polymorphic allele in a nucleic acid sample from a subject having a psychiatric disease or disorder. As used herein, a “psychiatric disease or disorder” may include, but is not limited to schizophrenia, bipolar disorder, and psychiatric depression.

Based on the polymorphic allele being identified in the subject, the subject may be identified as having responsiveness to an antipsychotic drug (APD), such as a typical APD or an atypical APD. As such, the subject may be treated by administering the APD to treat the psychiatric disorder and/or the symptoms thereof after the polymorphic allele has been identified.

Accordingly, the disclosed methods, kits, and devices optionally may utilize or include an antipsychotic drug (APD). Suitable APDs may include typical APDs and atypical APDs. APDs for use in the disclosed methods, kits, and devices, may include, but are not limited to Lurasidone (Latuda®), Clozapine (Clozaril®), Benperidol (Anguil®, Benguil®, Frenactil®, Glianimon®), Bromperidol (Bromodol®, Impromen®), Droperidol (Droleptan®, Inapsine®), Haloperidol (Haldol®, Serenace®), Moperone (Luvatren®), Pipamperone (Dipiperon®, Piperonil®), Timiperone (Celmanil®, Tolopelon®), Diphenylbutylpiperidine, Fluspirilene (Imap®), Penfluridol (Semap®), Pimozide (Orap®), Acepromazine (Plegicil®), Chlorpromazine (Largactil®, Thorazine®), Cyamemazine (Tercian®), Dixyrazine (Esucos®), Fluphenazine (Modecate®, Permitil®, Prolixin®), Levomepromazine (Levinan®, Levoprome®, Nozinan®), Mesoridazine (Lidanil®, Serentil®), Perazine (Peragal®, Perazin®, Pemazinum®, Taxilan®), Pericyazine (Neulactil®, Neuleptil®), Perphenazine (Trilafon®), Pipotiazine (Lonseren®, Piportil®), Prochlorperazine (Compazine®), Promazine (Prozine®, Sparine®), Promethazine (Avomine®, Phenergan®), Prothipendyl (Dominal®), Thioproperazine (Majeptil®), Thioridazine (Aldazine®, Mellaril®, Melleril®), Trifluoperazine (Stelazine®), Triflupromazine (Vesprin®), Chlorprothixene (Cloxan®, Taractan®, Truxal®), Clopenthixol (Sordinol®), Flupentixol (Depixol®, Fluanxol®), Tiotixene (Navane®, Thixit®), Zuclopenthixol (Acuphase®, Cisordinol®, Clopixol®), Clotiapine (Entumine®, Etomine®, Etumine®), Loxapine (Adasuve®, Loxitane®), Prothipendyl (Dominal®), Carpipramine (Defekton®, Prazinil®), Clocapramine (Clofekton®, Padrasen®), Molindone (Moban®), Mosapramine (Cremin®), Sulpiride (Meresa®), Sultopride (Bametil®, Topral®), Veralipride (Agreal®), Amisulpride (Solian®), Amoxapine (Asendin®), Aripiprazole (Abilify®), Asenapine (Saphris®, Sycrest®), Blonanserin (Lonasen®), Iloperidone (Fanapt®, Fanapta®, Zomaril®), Melperone (Buronil®, Buronon®, Eunerpan®, Melpax®, Neuril®), Olanzapine (Zyprexa®), Paliperidone (Invega®), Perospirone (Lullan®), Quetiapine (Seroquel®), Remoxipride (Roxiam®), Risperidone (Risperdal®), Sertindole (Serdolect®, Serlect®), Trimipramine (Surmontil®), Ziprasidone (Geodon®, Zeldox®), and Zotepine (Lodopin®, Losizopilon®, Nipolept®, Setous®). The APD may exhibit a number of biological activities including, but not limited to antagonism of one or more of the following sites, α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor. In some embodiments, the APD may exhibit at least partial agonism of the 5-HT_{1A} receptor. In some embodiments, the APD may exhibit negligible or no biological activity as a ligand for the H₁ receptor and/or mACh receptor (e.g., a Kᵢ>5 μM, 10 μM, 50 μM, 100 μM, or 500 μM). Suitable APD may include lurasidone, ziprasidone, clozapine, olanzapine, risperidone, perphenazine, and sertindole.

The disclosed methods, kits, and devices may utilize or include a reagent that is utilized for detecting a polymorphic allele. Suitable reagents may include nucleic acid reagents. For example, nucleic acid reagents may include reagents comprising a DNA oligonucleotide that hybridizes specifically to the polymorphic allele or that hybridizes specifically to a polymorphism in the polymorphic allele. In some embodiments, the methods, kits, and device may utilize or include nucleic acid reagents that comprise one or more primers for sequencing at least a portion of the polymorphic allele. In further embodiments, the methods, kits, and device may utilize or include nucleic acid reagents that comprise one or more primer pairs for amplifying at least a portion of the polymorphic allele.

The disclosed kits and/or devices disclosed herein may be assembled into systems for performing the methods disclosed herein. Manual and/or automated systems comprising the contemplated kits and/or devices are contemplated herein.

As used herein the terms “diagnose” or “diagnosis” or “diagnosing” refer to distinguishing or identifying a disease, syndrome or condition or distinguishing or identifying a person having or at risk for developing a particular disease, syndrome or condition. As used herein the terms “prognose” or “prognosis” or “prognosing” refer to predicting an outcome of a disease, syndrome or condition. The methods contemplated herein include diagnosing a psychiatric disorder in a subject that is associated with polymorphisms as disclosed herein. The methods contemplated herein also include determining a prognosis for a subject having a psychiatric disorder that is associated with the polymorphisms disclosed herein.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. In particular, the methods contemplated herein include treating a subject having or at risk for developing a psychiatric disorder that is associated with the polymorphisms disclosed herein.

The present methods may include detecting a polymorphism in a subject sample (e.g., a sample comprising nucleic acid). The term “sample” or “subject sample” is meant to include biological samples such as tissues and bodily fluids. “Bodily fluids” may include, but are not limited to, blood, serum, plasma, saliva, cerebral spinal fluid, pleural fluid, tears, lactal duct fluid, lymph, sputum, and semen. A sample may include nucleic acid, protein, or both.

The detected polymorphism is present in nucleic acid. The term “nucleic acid” or “nucleic acid sequence” refers to an oligonucleotide, nucleotide or polynucleotide, and fragments or portions thereof, which may be single or double stranded, and represents the sense or antisense strand. A nucleic acid may include DNA or RNA, and may be of natural or synthetic origin. For example, a nucleic acid may include mRNA or cDNA. Nucleic acid may include nucleic acid that has been amplified (e.g., using polymerase chain reaction). Nucleic acid may include genomic nucleic acid.

As used herein, the term “assay” or “assaying” means qualitative or quantitative analysis or testing.

As used herein the term “sequencing,” as in determining the sequence of a polynucleotide, refers to methods that determine the base identity at multiple base positions or that determine the base identity at a single position.

The term “amplification” or “amplifying” refers to the production of additional copies of a nucleic acid sequence. Amplification is generally carried out using polymerase chain reaction (PCR) technologies known in the art.

The term “oligonucleotide” is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

The present methods and kits may utilize or contain primers, probes, or both. The term “primer” refers to an oligonucleotide that hybridizes to a target nucleic acid and is capable of acting as a point of initiation of synthesis when placed under conditions in which primer extension is initiated (e.g., primer extension associated with an application such as PCR). For example, primers contemplated herein may hybridize to one or more polynucleotide sequences comprising the polymorphisms disclosed herein. A “probe” refers to an oligonucleotide that interacts with a target nucleic acid via hybridization. A primer or probe may be fully complementary to a target nucleic acid sequence or partially complementary. The level of complementarity will depend on many factors based, in general, on the function of the primer or probe. For example, probes contemplated herein may hybridize to one or more polynucleotide sequences comprising the polymorphisms disclosed herein. A primer or probe may specifically hybridize to a target nucleic acid (e.g., hybridize under stringent conditions as discussed herein). In particular, primers and probes contemplated herein may hybridize specifically to one or more polynucleotide sequences that comprise the polymorphisms disclosed herein and may be utilized to distinguish a polynucleotide sequence comprising a minor allele from a polynucleotide sequence comprising the major allele.

An “oligonucleotide array” refers to a substrate comprising a plurality of oligonucleotide primers or probes. The arrays contemplated herein may be used to detect the polymorphisms disclosed herein.

As used herein, the term “specific hybridization” indicates that two nucleic acid sequences share a high degree of complementarity. Specific hybridization complexes form under stringent annealing conditions and remain hybridized after any subsequent washing steps. Stringent conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art.

As used herein, a “target nucleic acid” refers to a nucleic acid molecule containing a sequence that has at least partial complementarity with a probe oligonucleotide, a primer oligonucleotide, or both. A primer or probe may specifically hybridize to a target nucleic acid.

The present methods may be performed to detect the presence or absence of the disclosed polymorphisms. Methods of determining the presence or absence of a polymorphism may include a variety of steps known in the art, including one or more of the following steps: reverse transcribing mRNA that comprises the polymorphism to cDNA, amplifying nucleic acid that comprises the polymorphism (e.g., amplifying genomic DNA that comprises the polymorphism), hybridizing a probe or a primer to nucleic acid that comprises the polymorphism (e.g., hybridizing a probe to mRNA, cDNA, or amplified genomic DNA that comprises the polymorphism), and sequencing nucleic acid that comprises the polymorphism (e.g., sequencing cDNA, genomic DNA, or amplified DNA that comprises the polymorphism).

A “polymorphism” refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism (SNP) is the replacement of one nucleotide by another nucleotide at the polymorphic site. Deletion of a single nucleotide or insertion of a single nucleotide also gives rise to single nucleotide polymorphisms. “Single nucleotide polymorphism” preferably refers to a single nucleotide substitution. Typically, between different individuals, the polymorphic site can be occupied by two different nucleotides which results in two different alleles with the most common allele in the population (i.e., the ancestral allele) being referred to as the “major allele” and the less common allele in the population being referred to as the “minor allele.” An individual may be homozygous or heterozygous for an allele of the polymorphism. Exemplary SNPs disclosed herein may include, but are not limited to SNPs present within a gene selected from the group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2. Polymorphisms can also encompass deletions and/or insertions, for example deletions and/or insertions within a gene selected from the group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

In the methods and kits, the minor allele and/or the major allele associated with a polymorphism may be detected. The methods may include and the kits and devices may be used for determining whether a subject is homozygous or heterozygous for a minor allele and/or major allele associated with a polymorphism (e.g., a SNP). The term “heterozygous” refers to having different alleles at one or more genetic loci in homologous chromosome segments. As used herein “heterozygous” may also refer to a sample, a cell, a cell population or a subject in which different alleles (e.g., major or minor alleles of SNPs) at one or more genetic loci may be detected. Heterozygous samples may also be determined via methods known in the art such as, for example, nucleic acid sequencing. For example, if a sequencing electropherogram shows two peaks at a single locus and both peaks are roughly the same size, the sample may be characterized as heterozygous. Or, if one peak is smaller than another, but is at least about 25% the size of the larger peak, the sample may be characterized as heterozygous. In some embodiments, the smaller peak is at least about 15% of the larger peak. In other embodiments, the smaller peak is at least about 10% of the larger peak. In other embodiments, the smaller peak is at least about 5% of the larger peak. In other embodiments, a minimal amount of the smaller peak is detected.

As used herein, the term “homozygous” refers to having identical alleles (e.g., major or minor alleles of SNPs) at one or more genetic loci in homologous chromosome segments. “Homozygous” may also refer to a sample, a cell, a cell population, or a subject in which the same alleles at one or more genetic loci may be detected. Homozygous samples may be determined via methods known in the art, such as, for example, nucleic acid sequencing. For example, if a sequencing electropherogram shows a single peak at a particular locus, the sample may be termed “homozygous” with respect to that locus.

The present methods may detect the polymorphism directly by analyzing chromosomal nucleic acid having the polymorphic variant sequence. Alternatively, the present method may detect the polymorphism indirectly by detecting an isoform nucleic acid expressed from the polymorphic variant sequence, by detecting an isoform polypeptide expressed from the polymorphic variant sequence, or by analyzing the expression of another nucleic acid or protein whose expression is regulated by the polymorphic sequence.

### ILLUSTRATIVE EMBODIMENTS

The following embodiments are illustrative and should not be interpreted to limit the scope of the claims subject matter.

### Embodiment 1

A method comprising: (a) detecting a polymorphic allele of polymorphism in a sample from a subject and/or receiving results of a test indicating that a subject has a polymorphic allele of a polymorphism, wherein the polymorphism is present in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions, and (b) administering an atypical antipsychotic drug to the subject after detecting the polymorphic allele and/or after receiving the results of the test.

### Embodiment 2

The method of embodiment 1, wherein the gene is selected from a group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

### Embodiment 3

The method of embodiment 1 or 2, wherein detecting and/or the test comprises amplifying at least a portion of the gene from the nucleic acid sample and detecting the polymorphism in the amplified portion.

### Embodiment 4

The method of any of the foregoing embodiments, wherein detecting and/or the test comprises sequencing at least a portion of the gene from the nucleic acid sample or from an amplicon obtained by amplifying at least a portion of the gene from the nucleic acid sample.

### Embodiment 5

The method of any of the foregoing embodiments, wherein detecting and/or the test comprises contacting nucleic acid comprising the polymorphism with a nucleic acid probe that hybridizes specifically to nucleic acid comprising the polymorphism.

### Embodiment 6

The method of any of the foregoing embodiments, wherein detecting and/or the test comprises determining whether the nucleic acid sample is homozygous for the polymorphism.

### Embodiment 7

The method of any of the foregoing embodiments, wherein detecting and/or the test comprises determining whether the nucleic acid sample is heterozygous for the polymorphism.

### Embodiment 8

The method of any of the foregoing embodiments, wherein the nucleic acid sample is obtained from blood or a blood product.

### Embodiment 9

The method of any of the foregoing embodiments, wherein the subject has a psychiatric disease or disorder selected from the group consisting of schizophrenia, bipolar disorder, and psychiatric depression.

### Embodiment 10

The method of any of the foregoing embodiments, wherein the subject has schizophrenia and is exhibited symptoms selected from the group consisting of positive symptoms, negative symptoms, cognitive symptoms, and any combination thereof.

### Embodiment 11

The method of any of the foregoing embodiments, wherein the APD is an atypical APD.

### Embodiment 12

The method of any of the foregoing embodiments, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

### Embodiment 13

The method of any of the foregoing embodiments, wherein the APD is an agonist or partial agonist for the 5-HT_{1A} receptor.

### Embodiment 14

The method of any of the foregoing embodiments, wherein the APD has negligible or no biological activity as a ligand for the H₁ receptor and/or mACh receptor (e.g., where the Kᵢ is >about 5 μM, 10 μM, 50 μM, 100 μM, or 500 μM).

### Embodiment 15

The method of any of the foregoing embodiments, wherein the atypical APD comprises lurasidone, ziprasidone, clozapine, olanzapine, risperidone, perphenazine, or serindole.

### Embodiment 16

A kit or combination comprising: (a) a nucleic acid reagent that hybridizes specifically to a polymorphic allele of a polymorphism in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions; and (b) an antipsychotic drug (APD).

### Embodiment 17

The kit or combination of embodiment 16, wherein the gene is selected from the group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

### Embodiment 18

The kit or combination of embodiment 16 or 17, wherein the kit further comprises one or more primer pairs for amplifying at least a portion of the gene.

### Embodiment 19

The kit or combination of embodiments 16-18, wherein the APD comprises an atypical APD.

### Embodiment 20

The kit or combination of any of embodiments 16-19, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

### Embodiment 21

The kit or combination of any of embodiments 16-20, wherein the APD is an agonist or partial agonist for the 5-HT_{1A} receptor.

### Embodiment 22

The kit or combination of any of embodiments 16-21, wherein the APD has negligible or no biological activity as a ligand for the H₁ receptor and/or mACh receptor (e.g., where the Ki is >about 5 μM, 10 μM, 50 μM, 100 μM, or 500 μM).

### Embodiment 23

The kit or combination of any of embodiments 16-22, wherein the atypical APD comprises lurasidone, ziprasidone, clozapine, olanzapine, risperidone, perphenazine, or serindole.

### Embodiment 24

A system comprising: (a) a device comprising a nucleic acid reagent that hybridizes specifically to a polymorphic allele of a polymorphism in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions; and (b) an antipsychotic drug (APD).

### Embodiment 25

The system of embodiment 24, wherein the gene is selected from the group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

### Embodiment 26

The system of embodiment 24 or 25, wherein the kit further comprises one or more primer pairs for amplifying at least a portion of the gene.

### Embodiment 27

The system of embodiments 24-26, wherein the APD comprises an atypical APD.

### Embodiment 28

The system of any of embodiments 24-27, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

### Embodiment 29

The system of any of embodiments 24-28, wherein the APD is an agonist or partial agonist for the 5-HT_{1A} receptor.

### Embodiment 30

The system of any of embodiments 24-29, wherein the APD has negligible or no biological activity as a ligand for the H₁ receptor and/or mACh receptor (e.g., where the Ki is >about 5 μM, 10 μM, 50 μM, 100 μM, or 500 μM).

### Embodiment 31

The system of any of embodiments 24-30, wherein the atypical APD comprises lurasidone, ziprasidone, clozapine, olanzapine, risperidone, perphenazine, or serindole.

### Embodiment 32

A method of treating schizophrenia in a subject in need thereof, the method comprising administering a therapeutic agent that increases expression and/or activity of RBFOX1.

### Embodiment 33

A method for identifying a therapeutic agent for treating schizophrenia, the method comprising screening a library of therapeutic agents for a therapeutic agent that increases expression and/or activity of RBFOX1 (A2BP1), and identifying a therapeutic agent that increases expression and/or activity of RBFOX1 (A2BP1) as the therapeutic agent for treating schizophrenia.

### Embodiment 34

A method comprising administering an antipsychotic drug (APD) to a subject having a psychiatric disease or disorder and the subject having a polymorphic allele in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions.

### Embodiment 35

The method of embodiment 34, wherein the subject has a psychiatric disease or disorder selected from the group consisting of schizophrenia, bipolar disorder, and psychiatric depression.

### Embodiment 36

The method of embodiment 34 or 35, wherein the subject has schizophrenia and is exhibited symptoms selected from the group consisting of positive symptoms, negative symptoms, cognitive symptoms, and any combination thereof.

### Embodiment 37

The method of any of embodiments 34-36, wherein the APD is an atypical APD.

### Embodiment 38

The method of any of embodiment s 34-37, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

### Embodiment 39

The method of any of embodiments 34-38, wherein the APD is an agonist or partial agonist for the 5-HT_{1A} receptor.

### Embodiment 40

The method of any of embodiments 34-39, wherein the APD has negligible or no biological activity as a ligand for the H₁ receptor and/or mACh receptor (e.g., where the Kᵢ is >about 5 μM, 10 μM, 50 μM, 100 μM, or 500 μM).

### Embodiment 41

The method of any of embodiments 34-40, wherein the atypical APD comprises lurasidone, ziprasidone, clozapine, olanzapine, risperidone, perphenazine, or serindole.

### Embodiment 42

The method of any of embodiments 34-41, wherein the subject has a polymorphic allele of a gene selected from RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

### Embodiment 43

The method of any claims 34-41, wherein the subject has a polymorphic allele of a polymorphism selected from: rs17596267, rs4854914, rs11223651, rs732381, rs133051, rs3759111, rs1222385, rs10897829, rs1402992, rs2237326, rsrs11120818, rs10484399, rs13207082, rs13198716, rs9467714, rs7749823, rs6940698, rs12058768, rs7551092, rs6720194, rs12479448, rs7581086, rs11898081, rs10169158, rs11919644, rs11713798, rs7615029, rs6787048, rs3904759, rs7666965, rs12513242, rs2169595, rs2575635, rs2077246, rs13106118, rs200031, rs4495147, rs17311549, rs17139218, rs17145587, rs3849067, rs2085575, rs6923772, rs11154344, rs10440955, rs2906199, rs1537593, rs17153105, rs2729547, rs13278546, rs17595134, rs6999334, rs9644441, rs1500318, rs10959577, rs7899847, rs17229781, rs12766218, rs12768287, rs1416924, rs11596919, rs7101596, rs11219139, rs11062907, rs7954760, rs9579835, rs12859383, rs7330675, rs12881028, rs16952671, rs11634382, rs16940273, rs16940448, rs7166706, rs7166722, rs294267, rs30012, rs4889551, rs16955317, rs225284, rs225255, rs7208758, rs1893243, rs10502610, rs585811, rs2819956, rs234324, rs2767607, rs17755028, rs17755054, rs6019817, rs2830909, rs742002, rs2160409, rs1531802, rs4027073, rs10069504, rs4865610, rs10474643, rs4712608, rs1005886, rs7743963, rs12208773, rs1168055, rs12719654, rs10091071, rs1495074, rs16879886, rs2512434, rs10505506, rs7010421, rs7017126, rs2468720, rs2169623, rs2447553, rs2093483, rs16909902, rs1805155, rs2282040, rs2282041, rs10512249, rs16909927, rs10512247, rs7042032, rs12255425, rs9971172, rs11187065, rs7919740, rs12241284, rs11192261, rs9783155, rs1507642, rs10768525, rs10837219, rs10837221, rs11035428, rs11035429, rs1676667, rs1702585, rs1676664, rs12364216, rs1940751, rs11600281, rs11110065, rs10860532, rs1387082, rs11110270, rs11110297, rs3887427, rs3884623, rs292462, rs4901072, rs8010726, rs12917416, rs2202979, rs28405182, rs9933246, rs9935875, rs9935962, rs9924951, rs9936248, rs10459843, rs11649628, rs726476, rs8048158, rs11077179, rs10468333, rs8045750, rs8057315, rs11641748, rs17674225, rs2965886, rs8048077, rs7195330, rs12597561, rs12950365, rs10512467, rs11653010, rs9960395, rs8093330, rs8110501, rs8131774, rs9980586, rs1699695, rs2828827, rs2828835, rs2832478, rs12835711, rs11120818, rs7101596, rs9644441, rs11596919, rs7551092, rs7239345, rs7166706, rs7166722, rs17595134, rs11588846, rs1816382, rs225255, rs2819166, rs11919644, rs9376913, rs16838, rs10440955, rs153479, rs11898081, rs12768287, rs1416924, rs9557996, rs10502610, rs1893243, rs41446249, rs2945908, rs2077246, rs6814341, rs7827390, rs17106441, rs10749378, rs11713798, rs6787048, rs2215381, rs1356374, rs12510684, rs7330675, rs7208758, rs7644745, rs12766218, rs11133186, rs2430807, rs9824811, rs321601, rs225284, rs13271251, rs6813301, rs1407066, rs2319068, rs503562, rs4748050, rs4750396, rs1500318, rs250585, rs9579835, rs403904, rs6500606, rs7243239, rs2575635, rs1980945, rs1676664, rs1702585, rs10837219, rs10837221, rs11035429, rs10512247, rs2169623, rs10512249, rs16909902, rs2282040, rs1676667, rs2447553, rs11035428, rs2282041, rs292462, rs1805155, rs292459, rs16975933, rs9524948, rs4601698, rs2468717, rs8110501, rs11110297, rs17118088, rs2160409, rs10768525, rs1507642, rs12126638, rs11192261, rs9783155, rs6717347, rs7204304, rs12929401, rs8010726, rs11591402, rs12364216, rs2391376, rs11110270, rs3887427, rs2003990, rs2745822, rs7313402, rs4076584, rs9341130, rs11808980, rs12241284, rs7919740, rs899073, rs17655606, rs3744635, rs2093483, rs2325882, rs402914, rs7946725, rs1908159, rs10171741, rs11649628, rs9935875, rs9935962, rs7302443, rs1206069, rs3884623, rs1032932, rs4964658, rs678697, rs4140729, rs6742598, rs3847178, rs1168055, rs10204599, rs2512434, rs4772812, rs2290273, rs11856774, rs16969710, rs11712608, rs16975932, rs10143206, rs7144971, rs6700661, rs10474643, rs12233091, rs8045750, rs17152573, rs17674225, rs10860532, rs11110065, rs2201331, rs976368, rs2832478, rs7973562, rs1387082, rs12447542, rs10500355, rs1057521725, rs1064794750, rs11643447, rs11645781, rs11866781, rs12444931, rs12446308, rs12921846, rs12926282, rs1478693, rs17139207, rs17139244, rs17648524, rs1906060, rs3785234, rs4124065, rs4146812, rs4786816, rs4787008, rs6500742, rs6500744, rs6500818, rs6500882, rs6500963, rs716508, rs7191721, rs7403856, rs7498702, rs870288, rs889699, rs9302841, rs9924951, rs1478697, rs4736253, rs10180106, rs511841, rs10895475, rs7017126, rs3857923, rs13270196, rs964441, rs13394481, rs1541947, rs16879886, rs11922361, rs2093483, rs524045, rs4733373, rs16879886, rs2239037, rs2007044, rs9636107, rs971215, and any combination thereof.

### Embodiment 44

The method of any of embodiments, 34-41, wherein the subject has a polymorphic allele of a polymorphism associated with RBFOX1 (A2BP1), optionally wherein the polymorphism is selected from rs17674225 (e.g., where the allele is G/T), rs8057315 (e.g., where the allele is C/A/G/T), rs726476 (e.g., where the allele is G/A/C/T), rs8045750 (e.g., where the allele is G/A), rs9924951 (e.g., where the allele is G/A), rs10468333 (e.g., where the allele is C/G/T), rs9933246 (e.g., where the allele is G/C/T), rs8048158 (e.g., where the allele is C/G), rs11077179 (e.g., where the allele is T/C), rs9936248 (e.g., where the allele is C/A), rs11641748 (e.g., where the allele is G/A), rs10459843 (e.g., where the allele is G/A/C), rs9935875 (e.g., where the allele is G/A/C), rs9935962 (e.g., where the allele is C/A), rs11649628 (e.g., where the allele is C/T), rs28405182 (e.g., where the allele is C/A/G/T), rs8048519 (e.g., where the allele is A/G), rs2159535 (e.g., where the allele is G/C), rs11077183 (e.g., where the allele is C/A), rs11077184 (e.g., where the allele is A/C/G), rs7198769 (e.g., where the allele is G/A/T), rs4786173 (e.g., where the allele is G/A), rs4141146 (e.g., where the allele is G/A), rs9935875 (e.g., where the allele is G/A), rs9935962 (e.g., where the allele is C/A), rs8057315 (e.g., where the allele is C/A/G/T), rs8045750 (e.g., where the allele is A/G), rs17674225 (e.g., where the allele is C/G/T), rs12447542 (e.g., where the allele is A/G), rs10500355 (e.g., where the allele is A/T), rs1057521725 (e.g., where the allele is A/G), rs1064794750 (e.g., where the allele is G/C), rs11643447 (e.g., where the allele is A/T), rs11645781 (e.g., where the allele is A/G), rs11866781 (e.g., where the allele is C/T), rs12444931 (e.g., where the allele is A/G), rs12446308 (e.g., where the allele is A/G), rs12921846 (e.g., where the allele is A/T, rs12926282 (e.g., where the allele is A/C), rs1478693 (e.g., where the allele is A//C), rs17139207 (e.g., where the allele is A/G), rs17139244 (e.g., where the allele is A/G), rs17648524 (e.g., where the allele is C/G), rs1906060 (e.g., where the allele is C/T), rs3785234 (e.g., where the allele is C/T), rs4124065 (e.g., where the allele is G/T), rs4146812 (e.g., where the allele is C/T), rs4786816 (e.g., where the allele is A/G), rs4787008 (e.g., where the allele is A/G), rs6500742 (e.g., where the allele is C/T), rs6500744 (e.g., where the allele is C/T), rs6500818 (e.g., where the allele is C/T), rs6500882 (e.g., where the allele is G/T), rs6500963 (e.g., where the allele is C/T), rs716508 (e.g., where the allele is C/T), rs7191721 (e.g., where the allele is A/G), rs7403856 (e.g., where the allele is A/G), rs7498702 (e.g., where the allele is C/T), rs870288 (e.g., where the allele is A/G), rs889699 (e.g., where the allele is A/G), rs9302841 (e.g., where the allele is A/T), rs9924951 (e.g., where the allele is A/G), rs1478697 (e.g., where the allele is A/G/T), and combinations thereof.

### Embodiment 45

The method, kit, combination, or system of any of the foregoing embodiments, wherein the subject is of European ancestry, African ancestry, East Asian ancestry, or Mexican ancestry.

### EXAMPLES

The following Example is illustrative and is not intended to limit the claimed subject matter.

### Example 1

Genetic Predictors of Antipsychotic Response to Lurasidone Identified in a Genome Wide Association Study and by Schizophrenia Risk Genes

Reference is made to Li et al., “Genetic predictors of antipsychotic response to lurasidone identified in a genome wide association study and by schizophrenia risk genes,” Schizophr. Res., 192 (2018) 194-204, 19 Apr. 2017, the content of which is incorporated herein by reference in its entirety.

Abstract

Biomarkers which predict response to atypical antipsychotic drugs (AAPDs) increases their benefit/risk ratio. We sought to identify common variants in genes which predict response to lurasidone, an AAPD, by associating genome-wide association study (GWAS) data and changes (Δ) in Positive And Negative Syndrome Scale (PANSS) scores from two 6-week randomized, placebo-controlled trials of lurasidone in schizophrenia (SCZ) subjects. We also included SCZ risk SNPs identified by the Psychiatric Genomics Consortium using a polygenic risk analysis. The top genomic loci, with uncorrected p<10⁻⁴, include: 1) synaptic adhesion (PTPRD, LRRC4C, NRXN1, ILIRAP1, SLITRK1) and scaffolding (MAGI1, MAGI2, NBEA) genes, both essential for synaptic function; 2) other synaptic plasticity-related genes (NRG1/3 and KALRN); 3) the neuron-specific RNA splicing regulator, RBFOX1; and 4) ion channel genes, e.g. KCNA10, KCNAB1, KCNK9 and CACNA2D3). Some genes predicted response for patients with both European and African Ancestries. We replicated some SNPs reported to predict response to other atypical APDs in other GWAS. Although none of the biomarkers reached genome-wide significance, many of the genes and associated pathways have previously been linked to SCZ. Two polygenic modeling approaches, GCTA-GREML and PLINK-Polygenic Risk Score, demonstrated that some risk genes related to neurodevelopment, synaptic biology, immune response, and histones, also contributed to prediction of response. The top hits predicting response to lurasidone did not predict improvement with placebo. This is the first evidence from clinical trials that SCZ risk SNPs are related to clinical response to an AAPD. These results need to be replicated in an independent sample.

Introduction

Antipsychotic drugs (APDs) are more effective to treat positive (psychotic) than negative symptoms or cognitive impairment in schizophrenia (SCZ). Psychotic symptoms respond to APDs in approximately 70% of patients with SCZ who may be classified as non-treatment resistant SCZ (non-TRS). The other ˜30% have moderate-severe positive symptoms after two or more trials with APDs and are referred to as treatment resistant SCZ (TRS) (Meltzer, 2012). Individual genetic, epigenetic, adherence, and other factors which affect drug absorption, metabolism, and interaction with various concomitant treatments account for the large variation in extent and time course of clinical response to APDs. Identifying multiple genetic and other biomarkers which contribute to these differences would facilitate optimal drug choice and might also lead to novel targets for APDs.

Lurasidone is a novel atypical APD with a relatively benign side effect profile (Bruijnzeel et al., 2015). Three Phase III registration trials showed it to be significantly better than placebo in improving total psychopathology in acutely psychotic SCZ patients, as measured by the change in total Positive And Negative Syndrome Scale (PANSS) scores (Loebel et al., 2013b; Meltzer et al., 2011a; Nasrallah et al., 2013a). Pharmacologically, lurasidone can be characterized as a more potent serotonin (5-HT)_{2A} than dopamine (DA) D₂ receptor blocker, a potent 5-HT₇ antagonist, and a direct acting 5-HT_{1A} partial agonist (Ishibashi et al., 2010). These pharmacologic features are the principal determinants of its efficacy and differentiation from both typical APDs, e.g. haloperidol, and other atypical APDs, e.g. risperidone (Huang et al., 2014). Reliable genetic biomarkers would help to identify the optimal patient population to be treated with this drug.

Previous pharmacogenetic (candidate gene studies) and pharmacogenomic [non-hypothesis driven genome-wide association (GWAS)] studies have reported predictors of response to other APDs (Arranz and Munro, 2011; Hamilton, 2015). A GWAS based on a well-defined and operationalized intermediate or (endo)phenotype such as change in positive symptoms in acutely psychotic patients, and controlled for ethnicity, because the genes involved may have larger effect sizes, can produce meaningful results using sample sizes in this range. By contrast, many tens of thousands of subjects per group may be required to identify genetic risks with only moderate effect sizes (odds ratio=1.1-1.2) in complex diseases, like SCZ, using unselected groups of SCZ patients (Consortium, 2014). The genetic risks for SCZ are subject to natural selection and those deleterious variants with bigger effect size are reduced in the population over time because of the low fitness in patients with SCZ. However, common variants associated with APD efficacy have been less affected by natural selection because APDs have been utilized only within the past 70 years. When a GWAS lacks the power to identify genetic variants as biomarkers for response because of individual small effect sizes, supplementary techniques which have been utilized here, are able to assist in identification of meaningful biomarkers. These include pathway analysis and polygenic risk scoring (Wang et al., 2010), and examining data from the most and least improved/worsened patient groups, omitting those with intermediate change scores (Lavedan et al., 2009).

Several small scale GWAS studies with identified pharmacogenomics biomarkers which predict response to APDs in SCZ have been reported. A GWAS of a phase III study of the atypical APD, iloperidone, in acutely psychotic patients identified six significant loci (Lavedan et al., 2009), one of which was a SNP near the genomic region of the 5-HT₇ receptor (HTR7). This is of special interest to this study because lurasidone is a 5-HT₇ receptor antagonist and this mechanism has been shown to be relevant to its ability to improve psychotic-like behavior and cognitive impairment in established rodent models (Galici et al., 2008). Next, the CATIE trial, an effectiveness trial in chronic SCZ patients (Lieberman et al., 2005), which randomized them to one of five APDs, was the basis for pharmacogenetic (Grossman et al., 2008; Need et al., 2009) and pharmacogenomic analyses (Adkins et al., 2011; McClay et al., 2011a; McClay et al., 2011b; Sullivan et al., 2009). Another pharmacogenomics study of Caucasian patients (n=89) with schizophrenia reported the top marker associated with improvement in positive symptom from olanzapine or risperidone monotherapy was in the HLA region (p=1.76×10⁻⁵) (Le Clerc et al., 2015). However, this SNP is not in linkage disequilibrium with SNPs identified by the PGC GWAS for genetic risk for SCZ. Recently, Stevenson et al. conducted an exploratory GWAS on antipsychotic response after 6-week treatment with risperidone in 86 first-episode patients with mixed ethnicities and diagnoses of SCZ, bipolar disorder, or major depression. SNPs inside a gene encoding glutamate receptor delta 2 (GRID2) were identified as the top markers (the lowest p=1.10×10⁻⁸) associated with the change score in Brief Psychiatric Rating Scale (BPRS) (Stevenson et al., 2016).

A genetic overlap between risk for SCZ and APD mechanism of action has been recently reported and advocated as a means to identify both APD drug targets and potential biomarkers (Ruderfer et al., 2016). Previous studies conducted by the PGC have shown that the polygenic risk derived from SCZ GWAS could be, in part, related to genetic effects on disorganized and negative symptoms, leading the authors to conclude that the identified genes, which included HLA region genes, might be treatment targets (Fanous et al., 2012). Another study showed that the polygenic risk scores (PRS) derived from PGC GWAS were higher in clozapine responders than clozapine non-responders (Frank et al., 2015), suggesting that these risk genes might be targets for clozapine. Thus, there is a naturally utilizing for risk genes to identify biomarkers for drug response in well-controlled small clinical trials.

We reported here the results of a GWAS which analyzed data from two clinical trials of lurasidone in acutely psychotic SCZ patients with European or African Ancestry (AA) (Meltzer et al., 2011b; Nasrallah et al., 2013b). We identified SNPs and pathways associated with change in PANSS total (ΔPANSS-T) and PANSS subscales which predicted efficacy and identified possible novel drug targets. We determined whether the top biomarkers belong to functional networks and their relationship to expression of the HTR₇ gene because of its important for the mechanism of action of lurasidone. Based on two polygenic modeling approaches, we tested whether the genetic variants from PGC GWAS significantly contributed to the variation of change in PANSS-total, positive, and negative subscales.

Methods and Materials

The clinical trials, subjects, and genotyping. The two clinical trials used for this analysis are both six-week, randomized, double-blind, lurasidone, placebo-controlled, multicenter registration trials, of DSM-IV acutely psychotic SCZ patients (Meltzer et al., 2011b; Nasrallah et al., 2013b). Patients who met TRS criteria were excluded. There were four fixed-dose treatment arms: lurasidone 40 and 120 mg/day, another atypical APD, olanzapine, 15 mg/day, and placebo (Pearl 1) (Meltzer et al., 2011b) and lurasidone 40, 80, and 120 mg/day, and placebo (Pearl 2) (Nasrallah et al., 2013b). The percentages of patients who achieved the a priori determined response: ≥20% improvement in ΔPANSS-T, was 61% in both the Pearl 1 and 2 studies. A total of 171/63 Caucasian and 131/54 AA for lurasidone/placebo-treated patients consented to participate in the genetic study. Ethnicity was validated as described below.

The data from the two clinical trials were analyzed together and genotyped using the Affymetrix 6.0 SNP Array (Affymetrix, Santa Clara, Calif., USA). Details of the method and Quality Control are provided in Supplemental material that accompany Li et al., “Genetic predictors of antipsychotic response to lurasidone identified in a genome wide association study and by schizophrenia risk genes,” Schizophr. Res., 192 (2018) 194-204, 19 Apr. 2017.

Evaluation of treatment response. The primary measure of efficacy, ΔPANSS-Total, was the difference between baseline and last observation carried forward (LOCF) for those with at least one PANSS rating after baseline. Results could be pooled across drug dosage arms because clinical change was not dose related (data not presented). The intention-to-treat (ITT) population included 157, 73, and 156 patients who received 40, 80, and 120 mg/day doses of lurasidone, respectively. Subjects within the 30th percentiles for greatest or least improvement in PANSS-Total (referred to as best and worst responders, hereafter) were included in a secondary analysis as previously done for iloperidone (Lavedan et al., 2009). The five PANSS factors: Positive, Negative, Disorganization, Excitement, and Anxiety/Depression, were shown to be present at baseline in this sample (available upon request).

Data Analysis.

DATA QC was conducted to exclude samples with MAF<0.05, genotyping rate<0.95, and significant deviation from HWE (p<0.0001). Principal component analysis (PCA) and association testing were conducted by PLINK 1.9 (Purcell et al., 2007). Linear regression with an additive model of minor alleles, adjusted for covariates, race, gender, and dosage, was utilized. False discovery rate (FDR) corrections for multiple testing were calculated using the Benjamini and Hochberg (BH) procedure. An unadjusted (without correction for multiple testing) p-value b 1.0×10⁻⁴ was arbitrarily set as the cutoff in the association test with ΔPANSS-T. SNP imputing was performed by IMPUTE2/SHAPEIT using 1000 genome phase 1 (EUR or AFR) as reference genome. Genes were annotated from genomic loci by scanDB. For identified loci in intergenic regions, the gene closest to the LD block within 250 MB was chosen as the annotated gene.

We performed pathway analysis using all SNPs (original and imputed) passing QC with p-value passing the cutoff. Multiple Association Network Integration Algorithm (GeneMANIA), text-mining to identify the interactome (GRAIL), functional prediction such as cis-eQTL (Braincloud), coexpression network (SEEK), protein-protein interaction (STRING), and tissue-specific gene expression (GTEx) were used for functional characterization and/or pathway(s) identification of the top GWAS hits.

Differential gene expression of identified gene markers. The expression of identified gene markers was evaluated in two independent gene expression datasets, GSE12649 and GSE21138, derived from post-mortem dorsolateral prefrontal cortex (DLPFC; BA46) of SCZ and control subjects, available at NCBI GEO. The differential gene expression was analyzed with the R Limma package. Details of the method are described in Supplemental material that accompany Li et al., “Genetic predictors of antipsychotic response to lurasidone identified in a genome wide association study and by schizophrenia risk genes,” Schizophr. Res., 192 (2018) 194-204, 19 Apr. 2017.

Polygenic Risk Modeling.

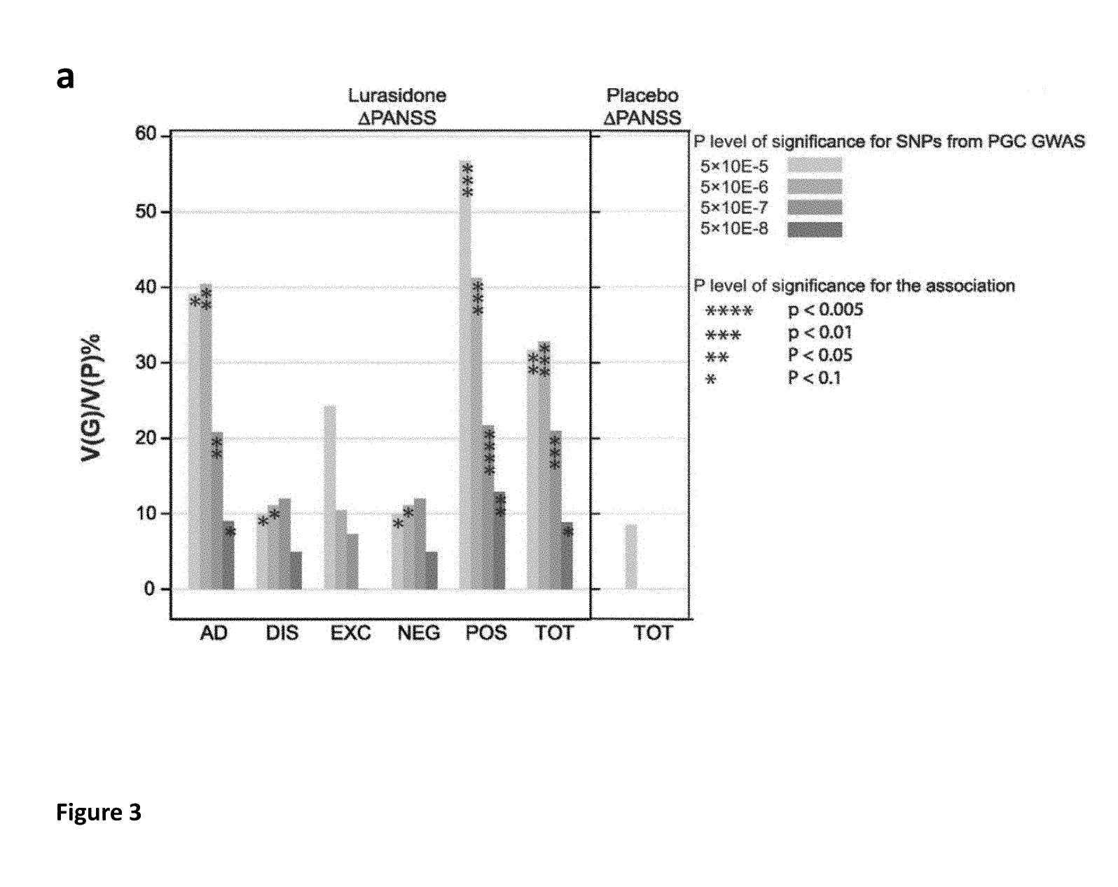

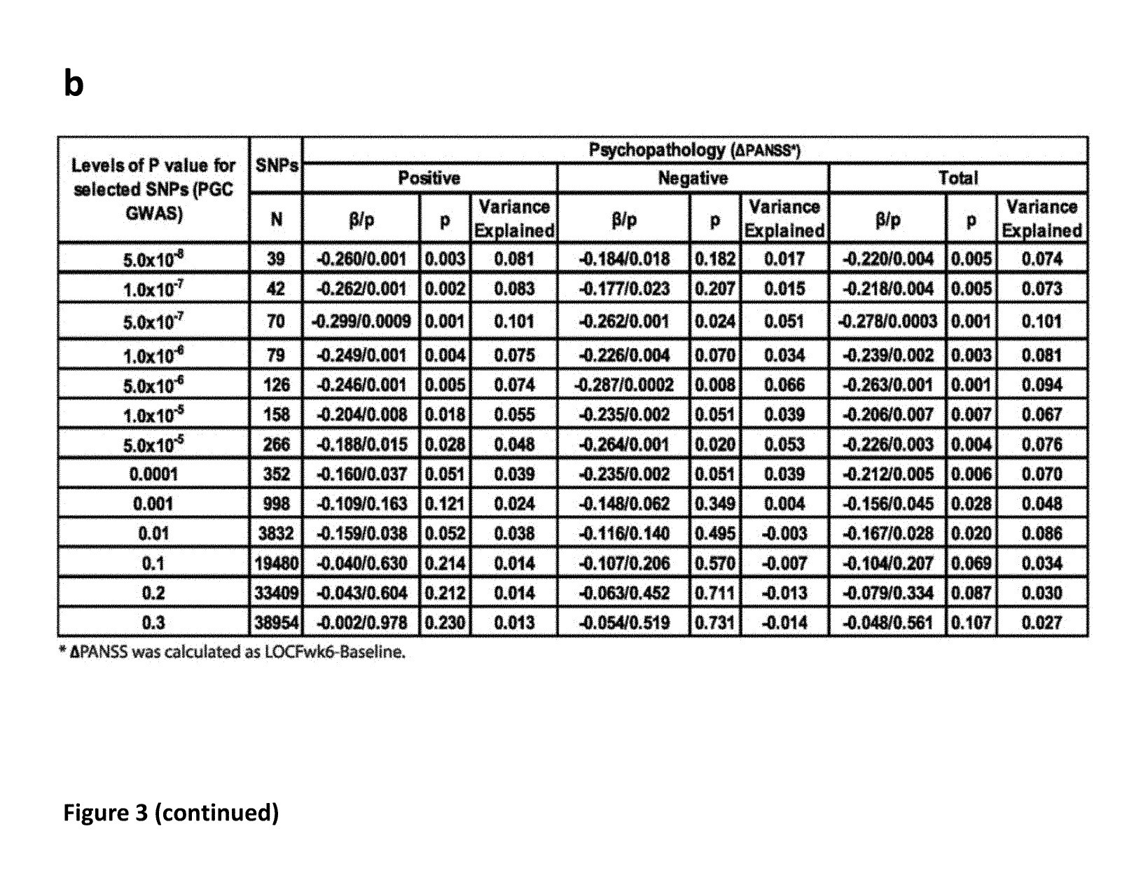

Polygenic risk was assessed with two popular polygenic modeling approaches, GCTA-GREML (a mixed-effects linear model) (Yang et al., 2016) and PLINK-Polygenic Risk Score (sum of the log odds) (Purcell et al., 2009). The polygenic risk modeling was only conducted on the Caucasian subjects from Pearl 1 and 2 (n=171). GCTA-GREML evaluated the regression relationship between the genetic and phenotypic similarity of pairs, after adjustment for covariates and relatedness of the subjects (genetic relationship matrix cutoff b0.05 for lurasidone and placebo samples). It can also be used to estimate specific components of heritability depending on the set of genetic markers. The estimation of the phenotypic variance explained by a subset of SNPs is presented as V(g)/V(p) (FIG. 3 and data not shown) and the effect size of individual SNPs was calculated by the best fitting effects of SNPs using a random effect model (BLUP) (data not shown). Polygenic Risk Score (PRS) was calculated based on the aggregated number of risk alleles identified from PGC GWAS (Consortium, 2014) after selection of SNPs at a step-wised p-level in the SNP-by-SNP association test, and then weighting the SNPs based on the log of Odds Ratio from PGC GWAS. The phenotypic variance of ΔPANSSLOCF was then predicted by logistic regression analysis of PRS plus covariates, including ancestry (PCs), gender, and dosage in the full model (Fanous et al., 2012). We downloaded the “Full SNP results” from the PGC “SCZ2 study”. This data includes imputed SNPs and their association with the risk for SCZ. Polygenic risk from two models was calculated based on the prune-in sets of SNPs after LD pruning of Pearl 1/2 Caucasian dataset by PLINK (−indep 50 5 1.5) as inclusion of correlated SNPs that do not contain independent signals can significantly reduce the predictive performance of models (Dudbridge, 2013).

Results

Population Stratification.

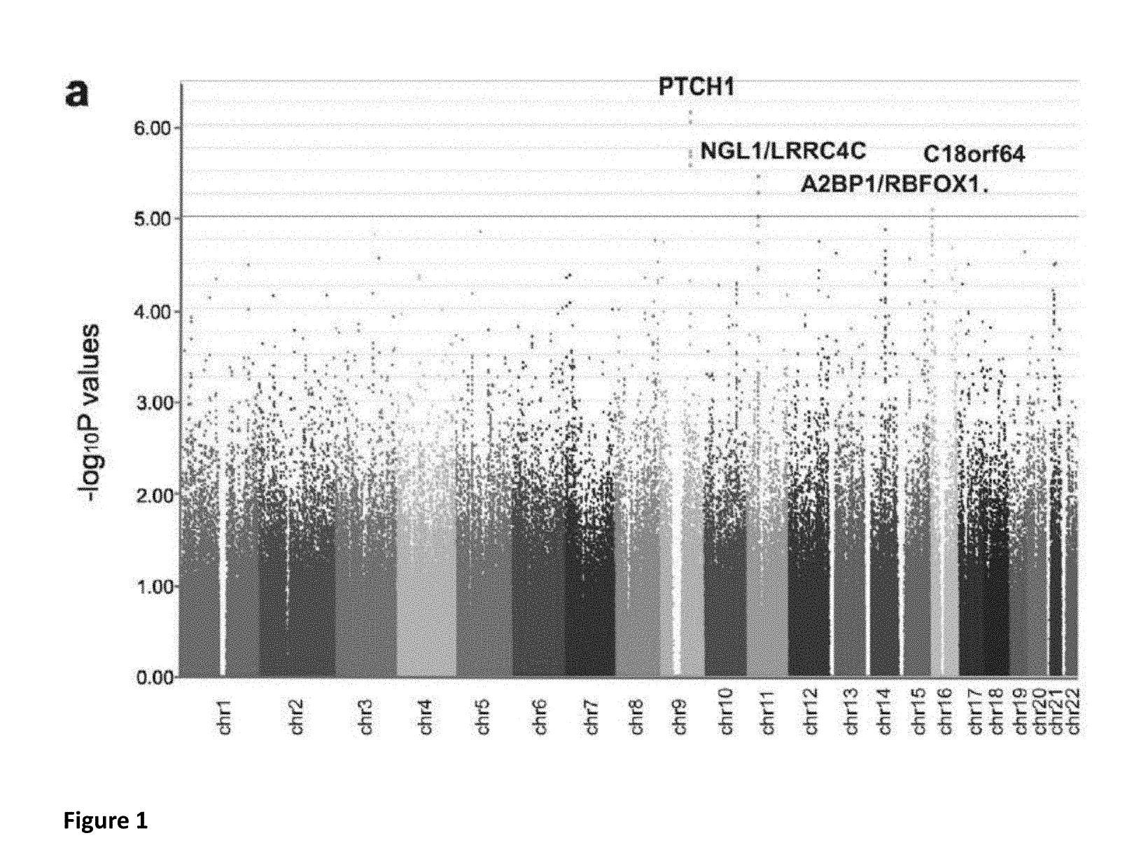

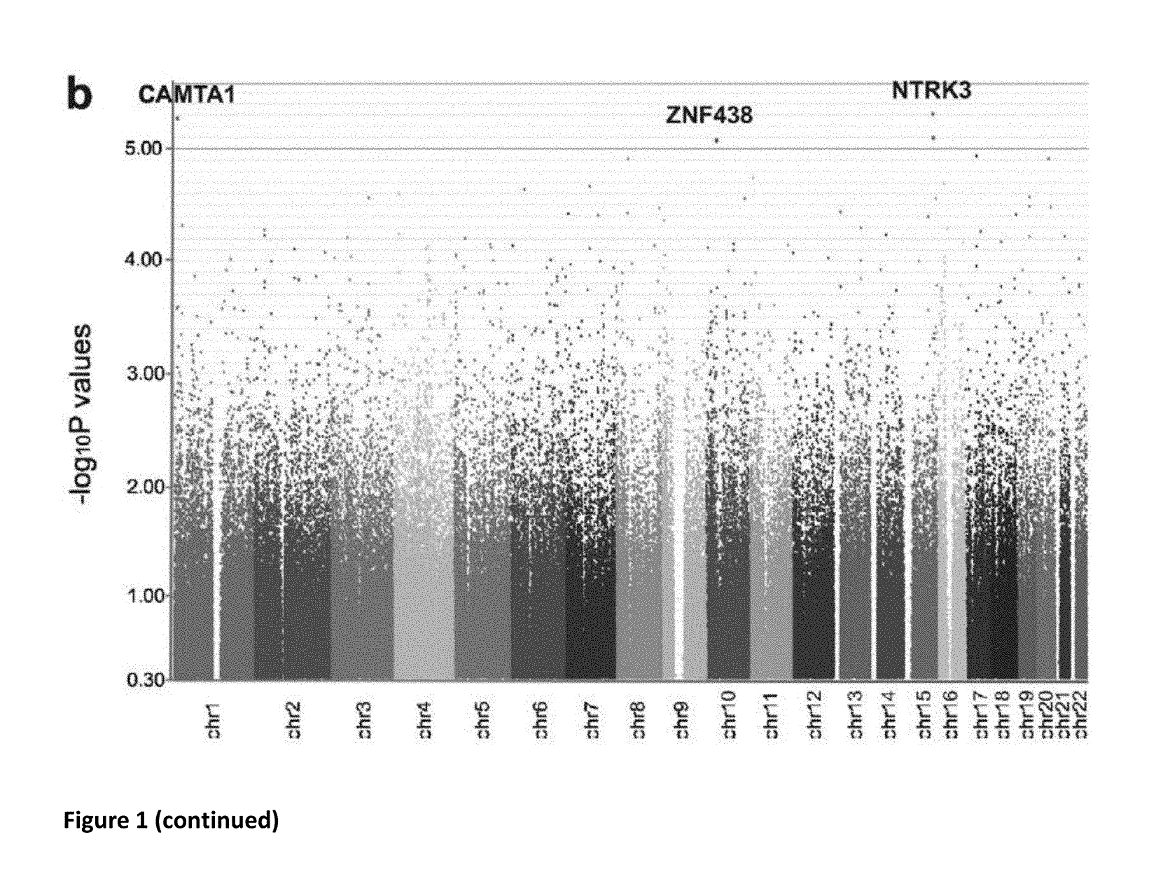

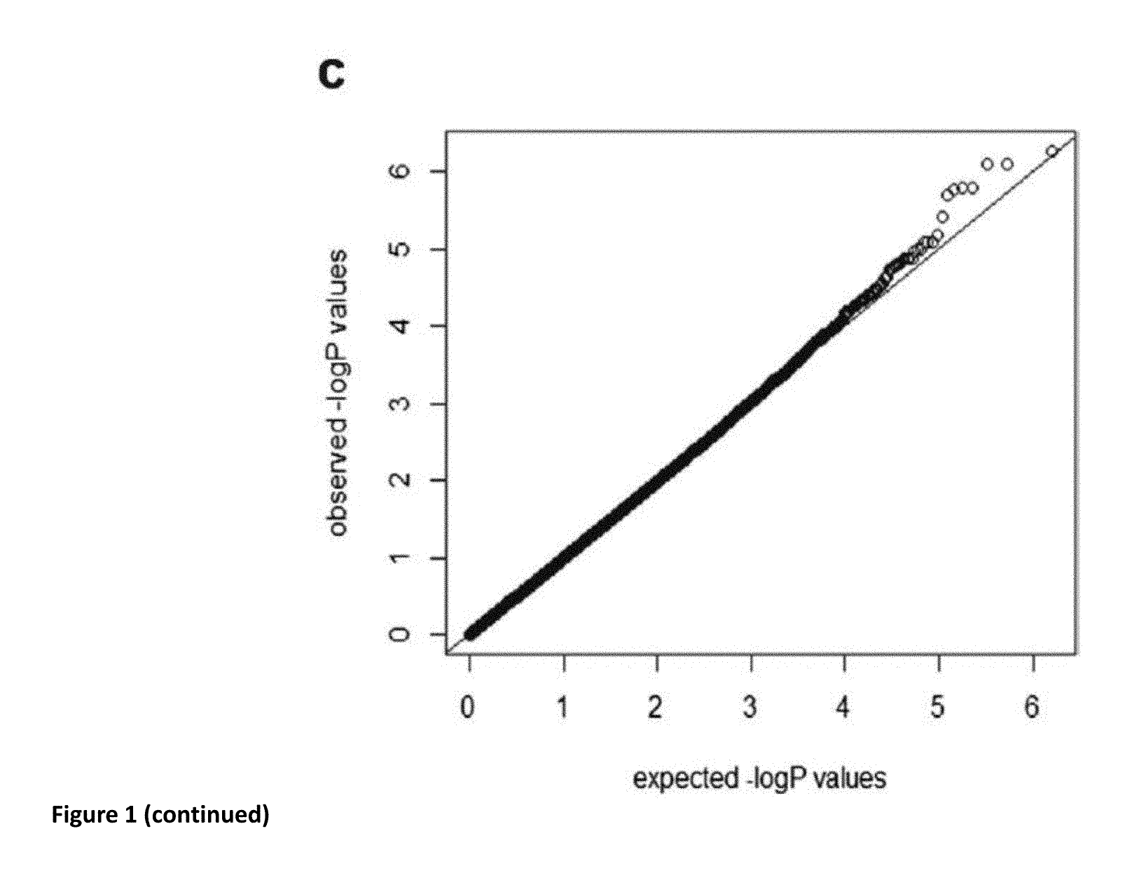

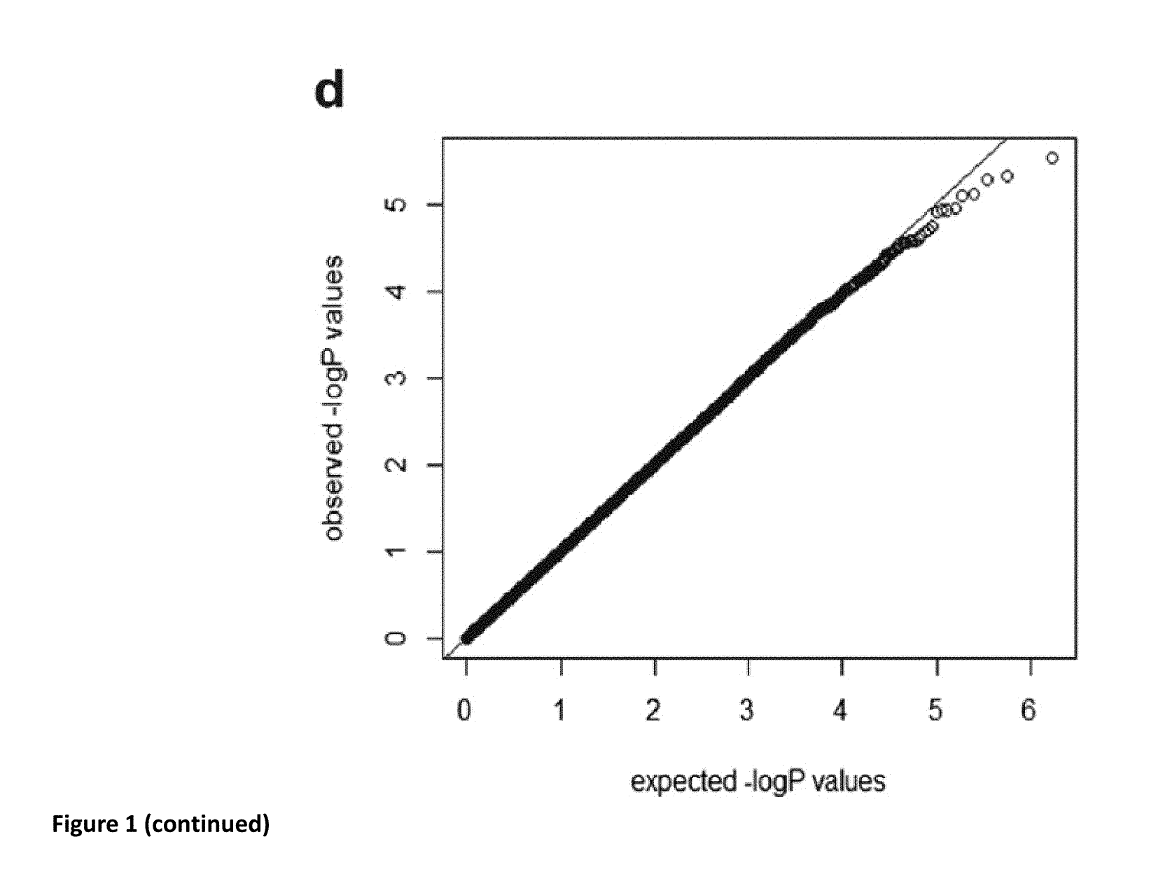

PCA revealed four major ethnic clusters: Caucasian, AA, East Asian, and Mexican (data not shown), matching the populations in 1000 Genome Phase 1. Population stratification and separation was employed throughout the following analyses. This process yielded 234 Caucasians, 171 and 63 in the lurasidone- and placebo-treated groups, respectively. There were 195 AAs, 131 and 54 in the lurasidone- and placebo groups, respectively. Adjustment using the top three PCs within each ethnic group was applied to the association tests. The QQ plots indicated no marked deviations of the observed distributions from the expected null distributions with genome inflation factor=1.00 for both ethnic groups (FIG. 1c, d). Both Kolmogorov-Smimov and Shapiro-Wilk tests indicated ΔPANSS-T followed the normal distribution with skewness and kurtosis indices between-1 and +1. Therefore, no subjects had to be excluded from the analysis. The demographic characteristics of each ethnic group are given in Table 1. ANOVA tests showed no significant difference in baseline total PANSS and ΔPANSS-T between the two groups.

TABLE 1
Clinical description of GWAS sample (n = 302)
of patients with schizophrenia treated with lurasidone.

| | | | African- |
|---|---|---|---|
| Ethnic group | | Caucasians | Americans |
| # of subjects (male/female) | | 171 (115/56) | 131 (99/32) |
| Study (Meltzer H Y et al./Nasrallah H. | | 119/52 | 80/51 |
| et al.) | | | |
| # of subjects in dosage (40/80/120 mg) | | 59/41/71 | 57/25/49 |
| Age (years) | | 40.6 ± 11.0 | 42.1 ± 10.0 |
| Days in study | All subjects | 32.8 ± 13.4 | 34.8 ± 12.3 |
| | Non-completers | 16.6 ± 9.1 | 17.0 ± 15.5 |
| # of enrolled (%) | Week 6 | 109 (63.7) | 91 (69.5) |
| | Week 5 | 114 (66.7) | 97 (74.0) |
| | Week 4 | 120 (70.2) | 102 (77.9) |
| | Week 3 | 136 (79.5) | 108 (88.4) |
| | Week 2 | 153 (89.5) | 118 (90.1) |
| | Week 1 | 163 (95.3) | 127 (96.9) |
| | Day 4 | 171 (100) | 131 (100) |
| CGI | Baseline | 4.9 ± 0.6 | 4.9 ± 0.7 |
| | Change (LOCF) | −1.0 ± 1.0 | −1.0 ± 1.0 |
| PANSS total | Baseline | 95.5 ± 8.8 | 94.6 ± 9.6 |
| | Change (LOCF) | −15.9 ± 17.0 | −18.0 ± 16.3 |
| PANSS positive | Baseline | 20.0 ± 3.1 | 20.5 ± 2.9 |
| | Change (LOCF) | −5.1 ± 4.9 | −5.3 ± 4.4 |
| PANSS negative | Baseline | 22.5 ± 4.6 | 22.3 ± 4.5 |
| | Change (LOCF) | −3.3 ± 4.8 | −4.2 ± 4.6 |
| PANSS | Baseline | 25.6 ± 4.2 | 24.1 ± 4.4 |
| disorganization | Change (LOCF) | −4.0 ± 4.2 | −4.0 ± 4.4 |
| PANSS excited | Baseline | 10.1 ± 3.0 | 10.5 ± 3.5 |
| | Change (LOCF) | −1.4 ± 3.7 | −1.2 ± 4.0 |
| PANSS anxiety/ | Baseline | 17.4 ± 3.5 | 17.2 ± 3.2 |
| depression | Change (LOCF) | −2.1 ± 4.2 | −3.3 ± 4.1 |

Association of genetic variants with treatment response. The top seven hits at the p<10⁻⁵ level for both Caucasians and AAs based on an initial linear regression analysis (MAF<0.05) with adjustment for the three covariates were PTCH1, NGL1 (also called LRRC4C), RBFOX1 (A2BP1), c18orf64 (see Manhattan plot in FIG. 1a), NTRK3, CAMTA1, and ZNF438 (see FIG. 1b). The top hits for each ethnic group with association p<10⁻⁴ were determined by preparing Circo plots (data not shown). Three genes, PTPRD,MAGI1, andCOL22A1/KCNK9, from the top-tier markers were significant (p<10⁻⁴) for both ethnic groups.

In order to enrich SNPs with biggest effect-size and determine their utility as potential biomarkers, we further examined the best and worst responders as previously defined (n=102/80 for Caucasian/AA). The second tract of the Circo plots illustrated the association of the markers derived from ΔPANSS-T for these cases. The individual associations between the top GWAS SNPs from ΔPANSS-T and improvement in the five PANSS subscales are reported in the third to the seventh tracts, which represent ΔPANSS-POS, ΔPANSS-NEG, ΔPANSS-DIS, ΔPANSS-AD, and ΔPANSS-EXC (data not shown). All the top SNPs from ΔPANSS-T were at least nominally associated with improvement in the five domains, and in the same direction. For example, our top genetic locus on RBFOX1 has a highly significant association (unadjusted p<0.001) with all five PANSS factors. On the other hand, the genetic locus on NRXN1 showed more variable association with the five factors, highest for negative symptoms (p=3.02×10⁻⁵) and least for anxiety/depression (p=0.08). The association of NRXN1 with ΔPANSS-T was stronger for the best responders than the entire sample. We also identified some genetic loci which showed increased association with ΔPANSS-T, exceeding the cutoff, p<10⁻⁴, in best and worst responders (data not shown), including MAGI2 (in Caucasians) and NBEA (in AAs), which have been previously linked to the risk for SCZ and/or Autism Spectrum Disorder (data not shown) (Castermans et al., 2010; Karlsson et al., 2012; Koide et al., 2012; Medrihan et al., 2009). It was noted that except for a genomic locus between SLC39A8 and NFKB1, the top hits identified in association with ΔPANSS-T in the lurasidone group showed either no significant association (p>0.05) with ΔPANSS-T in the placebo-treated group or the direction of the β weight for the minor allele was opposite to that in the lurasidone-treated group, suggesting that the top response markers were specific to lurasidone.

All significant associations at p<10⁻⁴ were located in noncoding regions. However, many affected gene expression and were inversely correlated to HTR7 expression, as will be discussed below. Genetic variants from the NRG1-(ERBB4)-KALRN signaling pathway (Penzes and Remmers, 2012) in Caucasians were individually associated with A PANSS-T, e.g. rs4733372/rs16879886/rs16879927/rs13266765 at intron region of NRG1 with unadjusted p=8.604×10⁻⁵. The identified SNPs in KALRN (i.e. rs1373606, rs7636024, rs13067494 and rs12636960, p=2.68×10-4 for ΔPANSS-T in Caucasians) are located in an intron region of Kalirin and serve as cis-eQTL only for Kalirin-7, the major isoform of KALRN, which has been identified as a key regulator of structural and functional plasticity of dendritic spines (Penzes and Remmers, 2012). Both NRG1 and KALRN are targets of RBFOX1 (Fogel et al., 2012), indicating that these pathways are highly interactive.

Functional Prediction and Clustering.

In order to predict the functional activity of the genetic variants, we utilized the gene expression database from DLPFC in Braincloud. More SNPs identified as top hits of Caucasians were potential cis-eQTLs than for those of AA patients (data not shown). GRAIL analysis suggested one of the functional categories predicting response consisted of ion channel genes, including KCNA10-KCNA2, KCNAB1, KCNK9, CACNA2D3, CACNA1S, and SCNN1B. GeneMANIA showed the functional categories with adjusted p-values<0.001 which predicted response to lurasidone were associated with “glutamate receptor activity” (GRIN2A, GRIK1, and NRXN1), “receptor clustering” (MAGI2, NRXN1), and “pre/post synaptic membrane organization” (PTPRD, IL1RAPL1, NRXN1) (data not shown).

Coexpression Analyses.

Coexpression enrichment analysis by SEEK was also used to functionally characterize the expression profiles of genes sharing common biological processes, function, or physical interaction. This is an indication of biological coherence in specific tissues. We meta-analyzed 34 gene expression datasets to identify the coexpression networks for PTPRD (data not shown) and RBFOX1 (data not shown). The top 500 coexpressed genes from 18,000 candidates, in red for positive, and blue for inverse correlation, with PTPRD, included a group of genes identified in this study as predictors of lurasidone response, e.g. NRXN1, NBEA, NRG1, FGF9, FUT8, MAGI1, NG1/LRRC4C, and SLITRK1. The genes coexpressed with PTPRD encode proteins forming synaptic adhesion complexes. The top ranked coexpressed genes for RBFOX1 include two previously identified SCZ targets, CAMTA1 and STXBP5L, with the putative binding motif, UGCAUGU, resulting in differential spliced exons in RBFOX1 knockout mice (Gehman et al., 2011). It is of particular interest that they were also the top hits associated with lurasidone response in AAs. Other lurasidone response markers reported to be RBFOX1-dependent genes (Fogel et al., 2012) are FGF9, DNAJA3, NRXN1, AP2B1, NTRK3, MAGI1, MAGI2, NBEA, GRID1, DLX2, and FBXO32. The coexpression of RBFOX1 and NRXN1 was tissue-specific (data not shown).

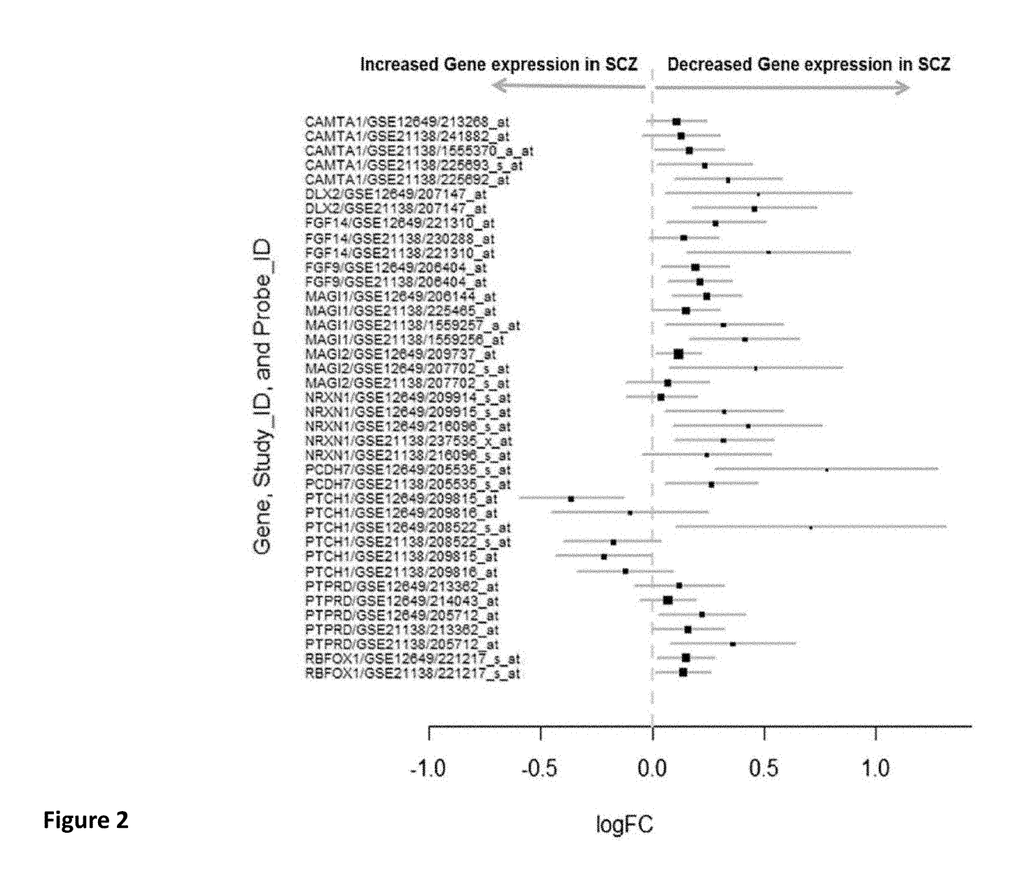

The HTR7-mediated signal transduction pathway has been reported to play an important role in the efficacy of lurasidone in multiple preclinical studies (Horiguchi et al., 2011; Huang et al., 2012; Ishibashi et al., 2010). Although we did not identify any genetic variants in the genomic region of HTR7 associated with treatment response to lurasidone, the expression of several GWAS-identified response predicting genes is significantly inversely correlated with expression of HTR7 in a brain region-specific manner (data not shown). Forty-five of the top 101 predictor genes show significant co-expression with HTR7, p<0.05. These include PTPRD, MAGI2, CAMTA1, FGF9, which are significantly decreased in SCZ patients, as shown in FIG. 2. The inverse correlations were most prominent in regions important for cognition: hippocampus, 9/13 (69.2%) and prefrontal cortex, 12/18 (66.7%).

Polygenic Risk Analysis.

Many of marker genes identified from this non-hypothesis-driven GWAS of PEARL 1 and 2 have been linked to SCZ or other psychiatric disorders (data not shown). Whether the genetic risk for SCZ identified by PGC GWAS, makes a significant contribution to treatment response to APDs has not been reported in prior pharmacogenomics studies. SNPs which individually show only modest association with treatment response, when combined, have potential to explain a significant portion of the variance. We, therefore, determined whether SCZ risk SNPs identified by the PGC GWAS, individually and collectively, had a significant effect on predicting lurasidone response. In the initial SNP by SNP association tests, some SNPs had nominal significant associations with ΔPANSS-TOTAL, −POS-NEG (p<0.05 or p<0.1) (data not shown). However, none of them survived Bonferroni correction. We next tested two polygenic modeling approaches: GCTA-GREML (a mixed-effects linear model) (Yang et al., 2016) and PLINK-PRS (sum of the log odds) (Purcell et al., 2009) using the Caucasian subjects from Pearl 1 and 2 studies (n=171). Both approaches showed that the genetic variants which had or were close to having genome-wide significant association with SCZ risk significantly contributed to the prediction of ΔPANSS-POS (FIGS. 3a and b). These SNPs also explained some of the variation in ΔPANSS-NEG. The SCZ risk genes which contributed to treatment response are related to neurodevelopment (TCF4, SOX2, and RBFOX1), synaptic biology (RBFOX1, IGSF9B, and CACNA1I), HLA, and histone. It is particularly noteworthy that rs12447542, located at RBFOX1, with p=1.122×10⁻⁶ as a marker for SCZ risk (PGC GWAS), was also related to treatment response to lurasidone (unadjusted p=0.046/0.096 for ΔPANSS_POS/ΔPANSS_NEG). Some of the risk SNPs which predicted lurasidone response were found to have a significant impact on gene expression and may be cis-eQTLs, according to Braincloud, BRAlNEAC or scanDB database in Caucasians. This include rs11222385 for response to lurasidone with unadjusted p=0.084/0.029 for ΔPANSS_POS/ΔPANSS_NEG as a cis-eQTL for SNX19 with p=4.1×10⁻²⁴ (BRAlNEAC). Both RBFOX1 and SNX19 are, therefore, of interest for understanding the mechanism of action of APDs. In support of this conclusion, SNX19 was recently identified by a novel method which integrated GWAS data from up to 339,224 individuals, and eQTL data from 5211 individuals as one of two prioritized genes related to SCZ (Zhu et al., 2016). rs749823, unadjusted p=0.0007 and 0.0096 for ΔPANSS_POS and ΔPANSS_NEG, respectively, located at HIST1H2BD, is a cis-eQTL for BTN3A2 (p=2.0×scanDB) and BTN3A3 (p=9.0×10⁻²⁴) and a methylation QTL for HIST1H4D (p=1.6×10⁻¹⁵, scanDB). Rs13198716 (unadjusted p=0.001 and 0.002 for ΔPANSS_POS and ΔPANSS_NEG, respectively) is a cis-eQTL for BTN3A2, BTN3A3, HLADQA1, HLA-DQA2, HIST1H4A/B/C/D/E/F/H/I/J/K/L, HIST2H4A/B and HIST4H4 (all p<4.0×10⁻⁵). GSF9B is a brain-specific adhesion molecule strongly expressed in GABAergic interneurons and it is coupled to neuroligin 2 via MAGI2, a top marker for lurasidone response, which has been shown to promote inhibitory synapse development (Woo et al., 2013). PRS with weighted effect size also showed results similar to GCTA-GREML (FIG. 3b). The negative value of 13 for SCZ PRS indicates acutely psychotic lurasidone responders after six weeks treatment have higher SCZ PRSs compared to lurasidone non-responders (the lowest p=0.002). We observed that risk alleles for SCZ in genes with bigger effect sizes are mostly associated with treatment response (data not shown). This is consistent with a previous study showing that the PRS for SCZ from the PGC GWAS were higher in clozapine responders compared to clozapine non-responders (Frank et al., 2015).

Replication of Top Hits from Previous Pharmacogenomics Studies.

As the five factor analysis of the PANSS reported for the CATIE study (McClay et al., 2011b) was shown to apply to this study as well, we compared the top SNPs for each PANSS factor from both studies. No population separation by ethnic groups was performed, as was the case for the CATIE study (McClay et al., 2011b). The MAFs between the CATIE and lurasidone trials are similar across all reported SNPs, because there was similar proportion of Caucasians in each study (57% in the lurasidone trial and 67% in CATIE). We replicated the top hits reported to be associated with positive symptom improvement in the ziprasidone group from the CATIE GWAS (Table 2). rs17390445 (p=0.02) and rs11722719 (p=0.0015), at the intergenic region close to PCDH7, proto-cadherin 7, were weakly associated with ΔPANSS-POS in both the CATIE (ziprasidone) and lurasidone datasets. After population stratification and separation of the lurasidone sample, we replicated a significant association in the AA population, e.g. rs17390445 (unadjusted p=0.05) and rs11722719 (p=0.005). In a reanalysis of two independent global gene expression datasets, we found the expression level of PCDH7 was significantly decreased in post-mortem tissue of the DLPFC in SCZ (FIG. 2). A candidate SNP, rs12122453, associated with treatment response to quetiapine for the PANSS anxiety/depression subscale, was also replicated in the lurasidone analysis for Caucasians.

TABLE 2
Summary of replication of some CATIE's top pharmacogenomic findings reported by McClay et al.

| Approach | APDs | Phenotype | SNP_ID | Gene | Minor allele | Race (N) | BETA | STAT | P |
|---|---|---|---|---|---|---|---|---|---|
| GWAS | Ziprasidone | ΔPOS | rs17390445 | near PCDH7 | T | ALL (301) | 0.976 | 2.426 | 0.016 |
| | | | | | C | AA (131) | −1.154 | −1.954 | 0.053 |
| | | | | | T | CEU (170) | 0.816 | 1.485 | 0.139 |
| GWAS | Ziprasidone | ΔPOS | rs11722719 | near PCDH7 | T | ALL (302) | 1.268 | 3.203 | 0.002 |
| | | | | | T | AA (131) | 1.602 | 2.844 | 0.005 |
| | | | | | T | CEU (171) | 0.900 | 1.639 | 0.103 |
| Candidate | Quetiapine | ΔAD | rs12122453 | FMO5 | C | ALL (301) | −0.562 | −1.62 | 0.106 |
| | | | | | C | AA (131) | 0.055 | 0.100 | 0.920 |
| | | | | | C | CEU (170) | −1.063 | −2.372 | 0.019 |
| Candidate | Perphenazine | ΔNEG | rs7829383 | NRG1 | G | ALL (302) | 0.929 | 1.973 | 0.049 |
| | | | | | G | AA (131) | 1.233 | 1.407 | 0.162 |
| | | | | | G | CEU (171) | 0.865 | 1.534 | 0.127 |

Another SNP, rs7829383, in NRG1, one of the top markers associated with negative symptom improvement with lurasidone, was borderline associated with ΔPANSS-NEG (unadjusted p=0.049) in the perphenazine-treated group in the CATIE candidate gene analysis (Need et al., 2009). ANKS1B, a top gene from the CATIE GWAS (McClay et al., 2011b), was also a top hit but with a different SNP (rs10860532 with unadjusted p=2.60×10⁻⁵) in this study.

Discussion

This study combined a non-hypothesis driven GWAS and recently identified genetic risk for SCZ with six week clinical trial data for lurasidone, an atypical APD, to identify a group of genetic biomarkers which significantly predicted improvement in PANSS Total and its five subscales, in Caucasian SCZ patients. Three genes, PTPRD, MAGI1, and COL22A1/KCNK9, from the top-tier markers were also significant (p<1e) in AA patients from the same trials. Top SNPs from the CATIE GWAS of other atypical APDs were partially replicated. SCZ risk SNPs from the PGC GWAS provided additional genetic biomarkers that predict response to lurasdione and provided the first evidence that SCZ risk SNPs from PGC GWAS are related to clinical response to an APD in a clinical trial. The key genes available to date identified by GWAS as predictors of response to lurasidone are related to synaptic development, plasticity, and maintenance, to ion channels, and are associated with cell and synaptic adhesion, scaffolding, and a key regulator of alternative splicing, which affects members of the previous two classes of proteins, among others. While none of the major hits reached genome wide significance or were from coding regions, many were eQTLs, providing further evidence that regulation of gene expression is likely to be a key mechanism of APD action (Martin et al., 2015) and further linked to SCZ by various types of evidence.

The clinical data utilized here are consistent with other studies of the efficacy of lurasidone in acutely psychotic patients, e.g. (Loebel et al., 2013a). The validity of these markers is supported by the finding that many of the annotated genes and enriched pathways which predict response to lurasidone have been previously implicated in treatment response to other APDs and/or the pathophysiology of SCZ (data not shown). We replicated several of the top biomarkers, e.g. NRG1, ˜PCDH7 and FMO5 reported in the CATIE pharmacogenomics study (McClay et al., 2011b). On the other hand, we did not replicate any of top six biomarkers found in a GWAS study of acutely psychotic patients treated with iloperidone (Lavedan et al., 2009). This could be due, in part, to pharmacologic differences between iloperidone and lurasidone, duration of observation and only a few markers reported from the iloperidone study. The finding that three genes, PTPRD, MAGI1, and COL22A1/KCNK9, from the most robust markers were significant for both ethnic groups provides additional support for their validity. That none of the hits identified here reached genome-wide significance is expected given the small sample size. This is a general limitation of pharmacogenomics studies. However, by choosing response to treatment during an acute exacerbation of psychosis rather than clinical change in relatively stable patients, with disease risk as the endophenotype, and selecting patients with the same ethnicity, the probability of finding reliable genetic biomarkers in a small sample was enhanced.

Relation of Response Genes to SCZ Pathophysiology.

Many of the annotated genes from the GWAS and enriched pathways which predicted response to lurasidone have been previously implicated in the pathophysiology of SCZ and/or treatment response to other APDs (data not shown). As will be discussed, this is particularly true for the synaptic adhesion genes, e.g. NRXN1, and the scaffolding genes, e.g., MAGI, MAGI2 (Karlsson et al., 2012; Koide et al., 2012). Functional gene network analysis also showed response to lurasidone was most strongly related to glutamate receptor activity, receptor clustering, and pre/post synaptic membrane organization. Our GWAS-based top markers include: 1)synaptic adhesion (NRXN1, PTPRD, LRRC4C, NTRK3, SLITRK1, IL1RAPL1, NCAM2, TSPAN13) and scaffolding genes (MAGI1, MAGI2, NBEA); [highlighted in a canonical signaling diagram (data not shown)] (Siddiqui and Craig, 2011; Takahashi and Craig, 2013; Um and Ko, 2013; Yamagata and Sanes, 2010); 2) alternative splicing genes, including RBFOX1 (Fogel et al., 2012); and 3) multiple ion channel genes, including a small group of potassium and calcium channel genes which play a key role in neurotransmission. Together, these genes and associated proteins organize synaptic composition, plasticity, and regulate the functional properties of excitatory and inhibitory synapses. Cell adhesion molecules are critical for cortical development, cognitive function (Yamagata and Sanes, 2010; Zheng et al., 2011), and synaptic maturation and plasticity (Danielson et al., 2012; Zheng et al., 2011). NRG1-ERBB4-KALRN-mediated, activity-dependent spine formation (Penzes and Remmers, 2012) and NMDARs postsynaptic complex (that is, GRIN2A, MAGI1, MAGI2) have been shown to be important to SCZ (Demontis et al., 2011; Karlsson et al., 2012; Koide et al., 2012). The scaffolding proteins have been particularly singled out for their role in the mechanism of action of atypical APDs, including clozapine (De Bartolomeis et al., 2013). MAGI1, MAGI2 (identified in the best and worst responders), and PTPRD were the top genes identified by GRAIL with the lowest pₜₑₓₜ (text-based GRAIL significance score) in rare or de novo deletions cases of SCZ and are functionally related to postsynaptic membrane/signaling complexes (Raychaudhuri et al., 2009). Although only three genetic regions (PTPRD, MAGI1, and COL22A1/KCNK9) from the top-tier markers were common to both ethnic groups, that the splicing targets of RBFOX1 apply to both ethnic groups is especially interesting. RBFOX1, a key regulator of neuron-specific alternative splicing in cell/synaptic adhesion molecules, has been repeatedly linked to SCZ, autism, and other neuropsychiatric disorders (Buizer-Voskamp et al., 2011; Melhem et al., 2011).

We previously reported that the synaptic adhesion gene, NRXN1, predicted improvement in psychopathology during clozapine treatment (Lett et al., 2011; Souza et al., 2010). These results were replicated by Jenkins et al. (Jenkins et al., 2014). NRXN1 neuroligins and related synaptic adhesion molecules could be promising targets for APD development.

HTR7.

There are many types of evidence linking HTR7 to lurasidone. HTR7 blockade would be expected to contribute to the ability of lurasidone to produce an antipsychotic effect (Galici et al., 2008). We have previously reported that HTR7 blockade is related to the ability of lurasidone to restore episodic memory in rats treated with subchronic phencyclidine (Horiguchi et al., 2011). A subeffective dose of the 5-HT₇ antagonist, SB269970, potentiated the ability of a subeffective dose of lurasidone to increase dopamine efflux in the mouse prefrontal cortex in awake freely moving mice (Huang et al., 2012). HTR7 blockade was shown to contribute to the ability of lurasidone to produce its antipsychotic effect in rodents (Galici et al., 2008). Lurasidone has been demonstrated to improve cognition in SCZ (Harvey et al., 2015) and improved the PANSS Cognitive subscale scores in the two studies included in this analysis. There is overlap between the genes which are associated with improvement in the PANSS Cognitive and Positive Subscale scores. The synaptic adhesion genes and other synaptic plasticity related genes which predicted response to lurasidone would also be expected to play a role in cognition. Thus, the genes which predict improvement in psychopathology in this study, e.g. NRXN1, could also be relevant to improvement in cognitive function (Mozhui et al., 2011). This inverse correlation between the expression of some lurasidone response genes and HTR7 suggests that the genes coexpressed with HTR7 could be downstream targets of HTR7 signaling.

Polygenic Risk Modeling.

For quantitative traits like treatment response, a number of SNPs individually show modest association, but when combined, may explain a significant portion of the variance in response. We assessed polygenic risk using two modeling approaches, GCTA-GREML (a mixed-effects linear model) (Yang et al., 2016) and PLINK-Polygenic Risk Score (sum of the log odds) (Purcell et al., 2009). We confirmed that genetic variants which have, or are close to having, genome-wide significant association with SCZ risk, contributed to the prediction of phenotypic variance in ΔPANSS-TOT, particularly in ΔPANSS-POS in Caucasian subjects. This conclusion did not hold for patients of African Ancestry (data not shown). The top SCZ risk genes which contributed to lurasidone response are related to neurodevelopment (TCF4, SOX2, and RBFOX1), synaptic biology (RBFOX1 and IGSF9B), HLAs, and histones. It is of interest that SNPs from the MHC region (approximately 26-33 Mb) identified as the top genetic risk genes for SCZ by PGC GWAS also contributed to prediction of treatment response to lurasidone. These SNPs have been previously reported as cis-eQTL for histone genes and HLA subtypes (Gejman et al., 2010), suggesting treatment response may be related to the epigenetic regulation of gene expression by histones and/or immunologic aspects of SCZ (Hasan et al., 2013).

Replication.

We replicated several of the top biomarkers reported in the CATIE pharmacogenomics study (Need et al., 2009). Non-replicated genetic markers of efficacy and side effects may be specific for only one rather than all APDs because of the unique pharmacologic properties of each APD. Some results from the CATIE study were replicated only when data from AA and Caucasians were combined, e.g. the biomarkers in NRG1. Other CATIE results were replicated when population stratification was conducted, e.g. the biomarkers near PCDH7 and FMO5. The other replication study we investigated was that for iloperidone in acutely psychotic SCZ patients. None of the top markers reported here in that study were replicated. However, this does not exclude the possibility of replication of biomarkers with smaller effect-sizes in other studies. Genome-wide meta-analysis examining data from studies with comparable design and patient populations, which tested multiple APDs, and employing population stratification and separation, may identify multiple biomarkers shared by more than one APD.

Conclusion.

In conclusion, we found that common genetic variants related to synaptic adhesion complexes, scaffolding, and the alternative splicing regulator, RBFOX1, are associated with treatment response to the atypical APD, lurasidone, in acutely psychotic SCZ patients. The combination of these genes with risk genes for SCZ as predictors of acute response to lurasidone strongly suggests that response to lurasidone in an acute exacerbation of the positive symptoms, and to other atypical APDs as well, targets the pathophysiology of SCZ that is also captured by risk genes for SCZ. The concordance of our findings with the risk genes for SCZ suggests that response to lurasidone and other atypical antipsychotic drugs could be related to the underlying pathology of the disorder.

We plan to test the GWAS results and SCZ risk SNPs from PGC GWAS utilizing data from another similarly designed trial for lurasidone which had the same entry criteria and the same method of assessing change in psychopathology (Loebel et al., 2013a). Such replication will provide an important test of whether or not small size studies of this type can provide meaningful information about choice of drugs for individual patients, drug targets, and the significance of SCZ risk genes. Supplementary data can be found online at dx.doi.org/10.1016/j.schres.2017.04.009 which accompanies Li et al., “Genetic predictors of antipsychotic response to lurasidone identified in a genome wide association study and by schizophrenia risk genes,” Schizophr. Res., 192 (2018) 194-204, 19 Apr. 2017.

### REFERENCES

Adkins, D. E., Åberg, K., McClay, J. L., Bukszár, J., Zhao, Z., Jia, P., Stroup, T. S., Perkins, D., McEvoy, J. P., Lieberman, J. A., 2011. Genomewide pharmacogenomic study of metabolic side effects to antipsychotic drugs. Mol. Psychiatry 16 (3), 321-332.
Arranz, M. J., Munro, J. C., 2011. Toward understanding genetic risk for differential antipsychotic response in individuals with schizophrenia. Expert. Rev. Clin. Pharmacol. 4 (3), 389-405.
Bruijnzeel, D., Yazdanpanah, M., Suryadevara, U., Tandon, R., 2015. Lurasidone in the treatment of schizophrenia: a critical evaluation. Expert. Opin. Pharmacother. 16 (10), 1559-1565.
Buizer-Voskamp, J. E., Muntjewerff, J.-W., Risk, G., Strengman, E., Sabatti, C., Stefansson, H., Vorstman, J. A., Ophoff, R A., 2011. Genome-wide analysis shows increased frequency of copy number variation deletions in Dutch schizophrenia patients. Biol. Psychiatry 70 (7), 655-662.
Castermans, D., Volders, K., Crepel, A., Backx, L, De Vos, R., Freson, K., Meulemans, S., Vermeesch, J. R., Schrander-Stumpel, C. T., De Rijk, P., 2010. SCAMPS, NBEA and AMISYN: three candidate genes for autism involved in secretion of large dense-core vesicles. Hum. Mol. Genet., ddq013 Consortium, S. W. G.o.t.P. G, 2014. Biological insights from 108 schizophrenia-associated genetic loci. Nature 511 (7510), 421-427.
Danielson, E., Zhang, N., Metallo, J., Kaleka, K., Shin, S. M., Gerges, N., Lee, S. H., 2012. SSCAM/MAGI-2 is an essential synaptic scaffolding molecule for the GluA2-containing maintenance pool of AMPA receptors. J. Neurosci. 32 (20), 6967-6980.
De Bartolomeis, A., Buonaguro, E., Iasevoli, F., 2013. Serotonin-glutamate and serotonin-dopamine reciprocal interactions as putativemolecular targets for novel antipsychotic treatments: from receptor heterodimers to postsynaptic scaffolding and effector proteins. Psychopharmacology 225 (1), 1-19.
Demontis, D., Nyegaard, M., Buttenschøn, Hedemand, A., Pedersen, C. B., Grove, J., Flint, T. J., Nordentoft, M., Werge, T., Hougaard, D. M., 2011. Association of GRIN1 and GRIN2A-D with schizophrenia and genetic interaction with maternal herpes simplex virus-2 infection affecting disease risk. Am. J. Med. Genet. B Neuropsychiatr. Genet. 156 (8), 913-922.
Dudbridge, F., 2013. Power and predictive accuracy of polygenic risk scores. PLoS Genet. 9 (3), e1003348.
Fanous, A. H., Zhou, B., Aggen, S. H., Bergen, S. E., Amdur, R. L., Duan, J., Sanders, A. R., Shi, J., Mowry, B. J., Olincy, A., 2012. Genome-wide association study of clinical dimensions of schizophrenia: polygenic effect on disorganized symptoms. Am. J. Psychiatry 169 (12), 1309-1317.
Fogel, B. L., Wexler, E., Wahnich, A., Friedrich, T., Vijayendran, C., Gao, F., Parikshak, N., Konopka, G., Geschwind, D. H., 2012. RBFOX1 regulates both splicing and transcriptional networks in human neuronal development. Hum. Mol. Genet. 21 (19), 4171-4186.
Frank, J., Lang, M., Witt, S., Strohmaier, J., Rujescu, D., Cichon, S., Degenhardt, F., Nothen, M., Collier, D., Ripke, S., 2015. Identification of increased genetic risk scores for schizophrenia in treatment-resistant patients. Mol. Psychiatry 20 (2), 150. Galici, R., Boggs, J. D., Miller, K. L., Bonaventure, P., Atack, J. R., 2008. Effects of SB-269970, a 5-HT7 receptor antagonist, in mouse models predictive of antipsychotic-like activity. Behav. Pharmacol. 19 (2), 153-159.
Gehman, Stoilov, P., Maguire, J., Damianov, A., Lin, C. H., Shiue, L, Ares Jr., M., Mody, I., Black, D. L., 2011. The splicing regulator Rbfox1 (A2BP1) controls neuronal excitation in the mammalian brain. Nat. Genet. 43 (7), 706-711.
Gejman, P. V., Sanders, A. R., Duan, J., 2010. The role of genetics in the etiology of schizophrenia. Psychiatr. Clin. N. Am. 33 (1), 35-66.
Grossman, I., Sullivan, P. F., Walley, N., Liu, Y., Dawson, J. R., Gumbs, C., Gaedigk, A., Leeder, J. S., McEvoy, J. P., Weale, M. E., 2008. Genetic determinants of variable metabolism have little impact on the clinical use of leading antipsychotics in the CATIE study. Genet. Med. 10 (10), 720-729.
Hamilton, S. P., 2015. The promise of psychiatric pharmacogenomics. Biol. Psychiatry 77 (1), 29-35.
Harvey, P. D., Siu, C. O., Ogasa, M., Loebel, A., 2015. Effect of lurasidone dose on cognition in patients with schizophrenia: post-hoc analysis of a long-term, double-blind continuation study. Schizophr. Res. 166 (1-3), 334-338.
Hasan, A., Mitchell, A., Schneider, A., Halene, T., Akbarian, S., 2013. Epigenetic dysregulation in schizophrenia:molecular and clinical aspects of histone deacetylase inhibitors. Eur. Arch. Psychiatry Clin. Neurosci. 263 (4), 273-284.
Horiguchi, M., Huang, M., Meltzer, H. Y., 2011. The role of 5-hydroxytryptamine 7 receptors in the phencyclidine-induced novel object recognition deficit in rats. J. Pharmacol. Exp. Ther. 338 (2), 605-614.
Huang, M., Horiguchi, M., Felix, A. R., Meltzer, H. Y., 2012. 5-HT1A and 5-HT7 receptors contribute to lurasidone-induced dopamine efflux. Neuroreport 23 (7), 436-440.
Huang, M., Panos, J. J., Kwon, S., Oyamada, Y., Rajagopal, L., Meltzer, H. Y., 2014. Comparative effect of lurasidone and blonanserin on cortical glutamate, dopamine, and acetylcholine efflux: role of relative serotonin (5-HT) 2A and DA D2 antagonism and 5-HT1A partial agonism. J. Neurochem. 128 (6), 938-949.
Ishibashi, T., Horisawa, T., Tokuda, K., Ishiyama, T., Ogasa, M., Tagashira, R., Matsumoto, K., Nishikawa, H., Ueda, Y., Toma, S., 2010. Pharmacological profile of lurasidone, a novel antipsychotic agent with potent 5-hydroxytryptamine 7 (5-HT7) and 5-HT1A receptor activity. J. Pharmacol. Exp. Ther. 334 (1), 171-181.
Jenkins, A., Apud, J. A., Zhang, F., Decot, H., Weinberger, D. R., Law, A. J., 2014. Identification of candidate single-nucleotide polymorphisms in NRXN1 related to antipsychotic treatment response in patients with schizophrenia. Neuropsychopharmacology 39 (9), 2170-2178.
Karlsson, R., Graae, L., Lekman, M., Wang, D., Favis, R., Axelsson, T., Gaiter, D., Belin, A. C., Paddock, S., 2012. MAGI1 copy number variation in bipolar affective disorder and schizophrenia. Biol. Psychiatry 71 (10), 922-930.
Koide, T., Banno, M., Aleksic, B., Yamashita, S., Kikuchi, T., Kohmura, K., Adachi, Y., Kawano, N., Kushima, I., Nakamura, Y., 2012. Common variants in MAGI2 gene are associated with increased risk for cognitive impairment in schizophrenic patients. PLoS One 7 (5), e36836.
Lavedan, C., Licamele, L., Volpi, S., Hamilton, J., Heaton, C., Mack, K., Lannan, R., Thompson, A., Wolfgang, C. D., Polymeropoulos, M. H., 2009. Association of the NPAS3 gene and five other loci with response to the antipsychotic iloperidone identified in a whole genome association study. Mol. Psychiatry 14 (8), 804-819.
Le Clerc, S., Taing, L., Fond, G., Meary, A., Llorca, P. M., Blanc, O., Beaune, P., Rajagopal, K., Jamain, S., Tamouza, R., Zagury, J. F., Leboyer, M., 2015. A double amino-acid change in the HLA-A peptide-binding groove is associated with response to psychotropic treatment in patients with schizophrenia. Transl. Psychiatry 5, e608.
Lett, T. A., Tiwari, A. K., Meltzer, H. Y., Lieberman, J. A., Potkin, S. G., Voineskos, A. N., Kennedy, J. L., Müller, D. J., 2011. The putative functional rs1045881 marker of neurexin-1 in schizophrenia and clozapine response. Schizophr. Res. 132 (2), 121-124.
Lieberman, J. A., Stroup, T. S., McEvoy, J. P., Swartz, M. S., Rosenheck, R. A., Perkins, D. O., Keefe, R. S., Davis, S. M., Davis, C. E., Lebowitz, B. D., 2005. Effectiveness of antipsychotic drugs in patients with chronic schizophrenia. N. Engl. J. Med. 353 (12), 1209-1223.
Loebel, A., Cucchiaro, J., Xu, J., Sarma, K., Pikalov, A., Kane, J. M., 2013a. Effectiveness of lurasidone vs. quetiapine XR for relapse prevention in schizophrenia: a 12-month, double-blind, noninferiority study. Schizophr. Res. 147 (1), 95-102.
Loebel, A., Cucchiaro, J., Xu, J., Sarma, K., Pikalov, A., Kane, J. M., 2013b. Effectiveness of lurasidone vs. quetiapine XR for relapse prevention in schizophrenia: a 12-month, double-blind, noninferiority study. Schizophr. Res. 147 (1), 95-102.
Martin, M N., Mimics, K., Nisenbaum, L. K., Vawter, M. P., 2015. Olanzapine reversed brain gene expression changes induced by phencyclidine treatment in non-human primates. Mol. Neuropsychiatry 1 (2), 82-93.
McClay, J. L., Adkins, D. E., Aberg, K., Bukszar, J., Khachane, A. N., Keefe, R. S., Perkins, D. O., McEvoy, J. P., Stroup, T. S., Vann, R. E., Beardsley, P. M., Lieberman, J. A., Sullivan, P. F., van den Oord, E. J., 2011a. Genome-wide pharmacogenomic study of neurocognition as an indicator of antipsychotic treatment response in schizophrenia. Neuropsychopharmacology 36 (3), 616-626.
McClay, J. L., Adkins, D. E., Aberg, K., Stroup, S., Perkins, D. O., Vladimirov, V. I., Lieberman, J. A., Sullivan, P. F., van den Oord, E. J., 2011b. Genome-wide pharmacogenomic analysis of response to treatment with antipsychotics. Mol. Psychiatry 16 (1), 76-85.
Medrihan, L., Rohlmann, A., Fairless, R., Andrae, J., airing, M., Missler, M., Zhang, W., Kilimann, M. W., 2009. Neurobeachin, a protein implicated in membrane protein traffic and autism, is required for the formation and functioning of central synapses. J. Physiol. 587 (21), 5095-5106.
Melhem, N., Middleton, F., McFadden, K., Klei, L., Faraone, S. V., Vinogradov, S., Tiobech, J., Yano, V., Kuartei, S., Roeder, K., Byerley, W., Devlin, B., Myles-Worsley, M., 2011. Copy number variants for schizophrenia and related psychotic disorders in Oceanic Palau: risk and transmission in extended pedigrees. Biol. Psychiatry 70 (12), 1115-1121.
Meltzer, H. Y., 2012. Clozapine: balancing safety with superior antipsychotic efficacy. Clin. Schizophr. Relat. Psychoses 6 (3), 134-144.
Meltzer, H. Y., Cucchiaro, J., Silva, R., Ogasa, M., Phillips, D., Xu, J., Kalali, A. H., Schweizer, E., Pikalov, A., Loebel, A., 2011a. Lurasidone in the treatment of schizophrenia: a randomized, double-blind, placebo- and olanzapine-controlled study. Am. J. Psychiatry 168 (9), 957-967.
Meltzer, H. Y., Cucchiaro, J., Silva, R., Ogasa, M., Phillips, D., Xu, J., Kalali, A. H., Schweizer, E., Pikalov, A., Loebel, A., 2011b. Lurasidone in the treatment of schizophrenia: a randomized, double-blind, placebo- and olanzapine-controlled study. Am. J. Psychiatr. 168 (9), 957-967.
Mozhui, K., Wang, X., Chen, J., Mulligan, M., Li, Z., Ingles, J., Chen, X., Lu, L., Williams, R., 2011. Genetic regulation of Nmx1 expression: an integrative cross-species analysis of schizophrenia candidate genes. Transl. Psychiatry 1 (7), e25.
Nasrallah, H. A., Silva, R., Phillips, D., Cucchiaro, J., Hsu, J., Xu, J., Loebel, A., 2013a. Lurasidone for the treatment of acutely psychotic patients with schizophrenia: a 6-week, randomized, placebo-controlled study. J. Psychiatr. Res. 47 (5), 670-677.
Nasrallah, H. A., Silva, R., Phillips, D., Cucchiaro, J., Hsu, J., Xu, J., Loebel, A., 2013b. Lurasidone for the treatment of acutely psychotic patients with schizophrenia: a 6-week, randomized, placebo-controlled study. J. Psychiatr. Res. 47 (5), 670-677.
Need, A. C., Keefe, R. S., Ge, D., Grossman, I., Dickson, S., McEvoy, J. P., Goldstein, D. B., 2009. Pharmacogenetics of antipsychotic response in the CATIE trial: a candidate gene analysis. Eur. J. Hum. Genet. 17 (7), 946-957.
Penzes, P., Remmers, C., 2012. Kalirin signaling: implications for synaptic pathology. Mol. Neurobiol. 45 (1), 109-118.
Purcell, S., Neale, B., Todd-Brown, K., Thomas, L., Ferreira, M. A., Bender, D., Maller, J., Sklar, P., De Bakker, P. I., Daly, Mi., 2007. PLINK: a tool set forwhole-genome association and population-based linkage analyses. Am. J. Hum. Genet. 81 (3), 559-575.
Purcell, S. M., Wray, N. R., Stone, J. L., Visscher, P. M., O'Donovan, M. C., Sullivan, P. F., Sklar, P., Ruderfer, D. M., McQuillin, A., Morris, D. W., 2009. Common polygenic variation contributes to risk of schizophrenia and bipolar disorder. Nature 460 (7256), 748-752.
Raychaudhuri, S., Plenge, R. M., Rossin, E. J., Ng, A. C., Purcell, S. M., Sklar, P., Scolnick, E. M., Xavier, R. J., Altshuler, D., Daly, M. J., 2009. Identifying relationships among genomic disease regions: predicting genes at pathogenic SNP associations and rare deletions. PLoS Genet. 5 (6), e1000534.
Ruderfer, D. M., Charney, A. W., Readhead, B., Kidd, B. A., Käller, A. K., Kenny, P. J., Keiser, M. J., Moran, J. L., Hultman, C. M., Scott, S. A., 2016. Polygenic overlap between schizophrenia risk and antipsychotic response: a genomic medicine approach. Lancet Psychiatry 3 (4), 350-357.
Siddiqui, T. J., Craig, A. M., 2011. Synaptic organizing complexes. Curr. Opin. Neurobiol. 21 (1), 132-143.
Souza, R. P., Meltzer, H. Y., Lieberman, J. A., Le Foll, B., Kennedy, J. L., 2010. Influence of neurexin 1 (NRXN1) polymorphisms in clozapine response. Hum. Psychopharmacol. 25 (7-8), 582-585.
Stevenson, J., Reilly, J., Harris, M., Patel, S., Weiden, P., Prasad, K., Badner, J., Nimgaonkar, V., Keshavan, M., Sweeney, J., 2016. Antipsychotic pharmacogenomics in first episode psychosis: a role for glutamate genes. Transl. Psychiatry 6 (2), e739.
Sullivan, P., Lin, D., Tzeng, J., van den Oord, E., Perkins, D., Stroup, T., Wagner, M., Lee, S., Wright, F., Zou, F., 2009. Genomewide association for schizophrenia in the CATIE study: results of stage 1. Mol. Psychiatry 14 (12), 1144.
Takahashi, H., Craig, A M., 2013. Protein tyrosine phosphatases PTPdelta, PTPsigma, and LAR: presynaptic hubs for synapse organization. Trends Neurosci. 36 (9), 522-534.
Urn, J. W., Ko, J., 2013. LAR-RPTPs: synaptic adhesion molecules that shape synapse development. Trends Cell Biol. 23 (10), 465-475.
Wang, K., Li, M., Hakonarson, H., 2010. Analysing biological pathways in genome-wide association studies. Nat. Rev. Genet. 11 (12), 843-854.
Woo, J., Kwon, S.-K., Nam, J., Choi, S., Takahashi, H., Krueger, D., Park, J., Lee, Y., Bae, J. Y., Lee, D., 2013. The adhesion protein IgSF9b is coupled to neuroligin 2 via S-SCAM to promote inhibitory synapse development. J. Cell Biol. 201 (6), 929-944.
Yamagata, M., Sanes, J. R., 2010. Synaptic localization and function of sidekick recognition molecules require MAGI scaffolding proteins. J. Neurosci. 30 (10), 3579-3588.
Yang, J., Lee, S. H., Wray, N. R., Goddard, M. E., Visscher, P. M., 2016. GCTA-GREML accounts for linkage disequilibrium when estimating genetic variance from genome-wide SNPs. Proc. Natl. Acad. Sci. 113 (32), E4579-E4580.
Zheng, C. Y., Seabold, G. K., Horak, M., Petralia, R. S., 2011. MAGUKs, synaptic development, and synaptic plasticity. Neuroscientist 17 (5), 493-512.
Zu, Z., Zhang, F., Hu, H., Bakshi, A., Robinson, M. R., Powell, J. E., Montgomery, G. W., Goddard, M E., Wray, N. R., Visscher, P. M., 2016. Integration of summary data from GWAS and eQTL studies predicts complex trait gene targets. Nat. Genet. 48 (5), 481-487.

### Example 2

RBFOX1/A2BP1 as Drug Target for Treatment of Schizophrenia and Other Disorders and Genetic Polymorphisms in RBFOX1/A2BP1 Predicting Treatment Response to Lurasidone

RNA binding protein, fox-1 homolog (C. elegans) 1, known as RBFOX1, A2BP1 or Fox-1, is a RNA-binding protein that regulates alternative splicing events by binding to 5′-UGCAUGU-3′ elements and regulates alternative splicing of tissue-specific exons and differentially spliced exons. Rbfox1 is specifically expressed in brain (neurons), heart and muscle according to GTEx Portal and controls neuronal excitation in the mammalian brain.

Accumulated evidence provides an extensive genetic connection between RBFOX1 and the etiology of multiple neurological and psychiatric diseases. Rare copy number variations (CNVs) in RBFOX1 have been associated with schizophrenia and autism. Rare (exonic) deletions have been linked to idiopathic generalized epilepsy, autism, and intellectual disability. Other structural variations have been identified in mental retardation, epilepsy and ADHD. Gain or Loss of function studies also has connected RBFOX1 to abnormal behavior and cognitive impairment.

Our recent study on Lurasidone pharmacogenomics reported that a number of genetic variants at RBFOX intronic regions are top markers associated with treatment response to Lurasidone, a potent serotonin 5-HT7 receptor antagonist, in schizophrenic patients in two combined clinical trials. The top ranked coexpression genes for RBFOX1 include two previously identified targets, CAMTA1 and STXBP5L with the putative binding motif, UGCAUGU, resulting in differential spliced exons in RBFOX1 knockout mice. Interestingly they were the top hits associated with treatment response to lurasidone in Afro-Americans. Other identified markers which are reported as RBFOX1-dependent genes are FGF9, DNAJA3, NRXN1, AP2B1, NTRK3, MAGI1, MAGI2, GRID1, DLX2, and FBXO32. The coexpression between RBFOX1 and NRXN1 was found to be tissue-specific. SNP×SNP interaction by conditional linear regression also provided evidence of genetic interaction. The significant association between rs2160409, a genetic variants of NRXN1, and ΔPANSS-T was decreased from p=8.11×10⁻⁵ to p=1.32×10⁻², with the biggest magnitude of increase of all the markers with p<0.001 after controlling for the RBFOX1 SNP, rs17674225 (SNPs locally in LD with rs17674225 were not included in the analysis). Some (NRXN1, FGF9, CAMTA1, PTPRD, DLX2, MAGI1, and MAGI2) have been reported to be significantly decreased in post-mortem tissue of SCZ, and others (PTPRD, CAMTA1, FGF9, and MAGI2) are significantly inversely correlated with HTR7 gene expression in brain areas relevant to schizophrenia. Most of them have previously linked to schizophrenia and/or cognition. This inverse correlation suggests that HTR7 may be negatively related to treatment response to lurasidone, and RBFOX1-related genes identified here could be the downstream target of HTR7 antagonism. Alternatively, lurasidone may exert its antipsychotic effect via suppression of HTR7-mediated, RBFOX1-dependent signaling and enhance the activity of the target genes in schizophrenic patients.

These findings have implications for developing novel drugs for treating schizophrenia based on high-throughput screening of small molecules which promote RBFOX1 expression and/or activity. These findings also have implications for the development of diagnostic tests to predict treatment response to APDs with HTR7 antagonism.

There is a huge unmet need for novel drug target(s) to treat psychosis, one of the major components of schizophrenia and other psychotic disorders. Although there are drugs that are effective for this, they are not effective in all patients and have a variety of side effects. There are also no existing biomarkers which facilitate the identification of patients who will or won't respond to them. We have identified RBFOX1 as a target to develop a small molecule that would be an effective antipsychotic in a variety of disorders, including schizophrenia, bipolar disorder and autism. Because of our information about linkage of RBFOX1 to other genes we believe it may be a useful treatment for autism and cognitive impairment. We believe SNPs identified in RBFOX1 will also be useful biomarkers based upon our discovery process.

### Example 3

Identifying the genetic risk factors for treatment response to lurasidone by genome-wide association study: A meta-analysis of samples from three independent clinical trials

Reference is made to the Li et al., “Identifying the genetic risk factors for treatment response to lurasidone by genome-wide association study: A meta-analysis of samples from three independent clinical trials,” Schizophr. Res. 2018 September; 198: 203-213, epub May 2, 2018, the content of which is incorporated by reference herein in its entirety.

Abstract

A genome-wide association study (GWAS) of response of schizophrenia patients to the atypical antipsychotic drug, lurasidone, based on two double-blind registration trials, identified SNPs from four classes of genes as predictors of efficacy, but none were genome wide significant (GWS). After inclusion of data from a third lurasidone trial, meta-analysis identified a GWS marker and other findings consistent with our first study. The primary end-point was change in Total Positive and Negative Syndrome Scale (PANSS) between baseline and last observation carried forward. rs4736253 (C/T single nucleotide variation on chromosome 8), a genetic locus near KCNK9, encoding the K_{2P}9.1 potassium channel, with a role in cognition and neurodevelopment, was the top marker in patients of European ancestry (EUR) (n=264), reaching GWS (p=4.78×10⁻⁸). rs10180106 (A/G single nucleotide variation on chromosome 2) (p=4.92×10⁻⁷), located at an intron region of CTNNA2, a SCZ risk gene important for dendritic spine stabilization, was one of other best response markers for EUR patients. SNPs at STXBP5L (rs511841, A/G single nucleotide variation on chromosome 3, p=2.63×10⁻⁷) were the top markers for patients of African ancestry (n=158). The association between PTPRD, NRG1, and MAGI1 previously reported to be related to response to lurasidone in the first two trials, showed a trend of significant association in the third trial. None of these genetic loci showed significant associations with clinical response in the corresponding placebo groups (n=107 for EUR; n=58 for AFR). This meta-analysis yielded the first GWAS-based GWS biomarker for lurasidone response and additional support for the conclusion that genes related to synaptic biology and/or risk for SCZ are the strongest predictors of response to lurasidone in schizophrenia patients.

Introduction

There is much variation in the response to antipsychotic drugs (APDs) in patients with schizophrenia (SCZ), leading to multiple trials to obtain desired response. Identification of biomarkers which predict response to APDs is beneficial to patients since failed trials increase the cost of treatment and prolong patient distress. The identification of reliable genetic marker for APDs based on clinical trials has been unsuccessful for many reasons, including relatively small sample sizes for association studies. These sizes, approximately 200 patients per group, contrasts with the sample sizes which have proven necessary to identify risk genes for SCZ (Ripke et al., 2014). Previous reports rarely examine both pharmacodynamic and pharmacokinetic factors, both of which contribute to response (Charlab and Zhang, 2013). Nevertheless, pharmacogenetic (Arranz and de Leon, 2007) and pharmacogenomics studies, alone or combined (Stevenson et al., 2016), have identified some biomarkers for APD response. For example, the NIMH-sponsored CATIE study which collected pharmacogenomics data from five APDs, one of which, perphenazine, a typical APD, differs significantly in mechanism of action from the other four atypical APDs (AAPDs) clozapine, olanzapine, risperidone, and ziprasidone (McClay et al., 2011a; McClay et al., 2011b; Need et al., 2009; Sullivan et al., 2009). There are a few pharmacogenomics studies of individual APDs, including iloperidone (Lavedan et al., 2009), lurasidone (Li et al., 2018), risperidone (Sacchetti et al., 2017), and paliperidone (Li et al., 2017), as well as one other with multiple APDs combined (Le Clerc et al., 2015). Two studies (Li et al., 2017; Stevenson et al., 2016) have identified genome-wide significant (GWS, p<5×10⁻⁸) markers, which were not replicated at the time of initial publication. Some of the reported GWS markers not GWS have been replicated, but did not reach GWS (Li et al., 2018; Sacchetti et al., 2017).

Schizophrenia risk genes have been reported by us (Li et al., 2018) and others to be among the predictors of clinical response to AAPDs. Most genes reported to predict response to APDs are related to synaptic transmission, calcium channels, pruning of synaptic spines, transcriptional factors, or known APD targets, e.g. DRD2, the gene which encodes dopamine (DA) D2 receptors (Ripke et al., 2014). An exome-sequencing study has demonstrated a polygenic burden of rare variants from these pathways which contributes to the risk for SCZ (Purcell et al., 2014). Gene-Sets Enrichment Analyses of rare and common variants identified in the PGC samples highlight known APD targets and novel predictors, including CACNA1C, GRIN2A, AKT3, and HCN1. These results suggest some genes that contribute to risk for SCZ and its pathogenesis may also contribute to the mechanism of APDs action (Ruderfer et al., 2016). However, there is no evidence that their contribution is greater than that of the non-risk genes. Thus, there is a need to identify specific biomarkers for individual APD, although some of these will be predictive of efficacy for more than one APD.

Lurasidone is an AAPD approved for treatment of SCZ and bipolar disorder (Meyer et al., 2009; Sanford, 2013). It shares some pharmacology with other AAPDs, i.e. more potent serotonin (5-HT)_{2A} than DA D₂ receptor antagonism (Meltzer, 2012); it is also a potent 5-HT₇ antagonist and 5-HT_{1A} partial agonist (Ishibashi et al., 2010), both of which may be relevant to its efficacy (Ishibashi et al., 2010; Meltzer et al., 2011b). We recently reported a pharmacogenomic study using data from PEARL 1 and 2, two double-blind, randomized, placebo-controlled phase 3 clinical trials in acutely psychotic SCZ patients (Li et al., 2018). The top predictors included common variants in multiple genes related to cell and synaptic adhesion and scaffolding proteins, ion channels, and an alternative splicing regulator, RBFOX1, in patients with European (EUR) or African (AFR) ancestry. Many response genes identified in that study, e.g. PTPRD, NRXN1, NRG1, MAGI1, had previously been associated with SCZ or other psychiatric disorders, or APD actions.

To test the robustness of the findings in that study, DNA from a third lurasidone clinical trial, PEARL 3 (Loebel et al., 2013b), which utilized the same inclusion and exclusion criteria as PEARL 1 and 2 trials, was included in a meta-analysis. The goal of this study was to validate or not the results of our previous pharmacogenomics study of response to lurasidone by combining samples in a meta-analysis and to seek GWS markers.

Materials and Methods

Participants. The three clinical trials (Loebel et al., 2013a; Meltzer et al., 2011a; Nasrallah et al., 2013) used in this analysis were six-week, randomized, double-blind, placebo-controlled, multicenter registration trials, of DSM-IV acutely psychotic SCZ patients. The primary measure of efficacy, ΔPANSS-TOT, was the difference between baseline and last observation carried forward (LOCF) for those with at least one PANSS rating at, or after two weeks of treatment, (ΔPANSS-TOT_{LOCF6WK}=PANSSTOT_{LOCF6WK}−PANSS-TOT_{Baseline}). The overall mean percentage of patients who achieved a priori determined response: >20% improvement in ΔPANSS-TOT_{LOCF6WK}, was 61% in the PEARL 1, 2 and 3 studies. A total of 587 subjects with valid treatment response data and verified race from two ethnic populations, EUR and AFR, gave written informed consent to participate in genetics study (368 of 545 (67.5% with EUR ancestry and 219 of 403 (54.3% with AFR ancestry). These trials included a small number of patients randomized to treatment with olanzapine (PEARL 1) or quetiapine (PEARL 3). There were too few subjects with genetic data to include them as a separate group in this analysis.

Genotyping and Population Stratification.

Genome-wide genotyping data were generated with Illumina Omni5Exome-4v1beadchip. Principal Component Analysis (PCA) revealed that self-identified EUR or AFR subjects from the three clinical trials clustered with EUR or AFR from the 1KG sample of the reference genome (data not shown). The samples (n=165/124 for EUR/AFR) from the lurasidone group of the PEARL 1 and 2 trials had been genotyped with Affymetrix 6.0 SNP array (n=171/131 for EUR/AFR). The concordance rates of the genotypes reported from each array platform were observed to be >99.95% across all SNPs (data not shown).

The demographic characteristics of the patients for each ethnicity are given in Table 3A for EUR and Table 3B for AFR.

TABLE 3
Demographic data for GWAS subjects selected from 3 clinical trials of Lurasidone and matched placebo by race. Table 3A, patients with
European Ancestry; Table 3B, patients with African Ancestry.

| | | | | | | | | |
|---|---|---|---|---|---|---|---|---|
| | | | P value for | P value | | | P value for | P value |
| | | | Chi-Square | for | | | Chi-Square | for |
| Clinical Trials | Pearl 1, 2 | Pearl 3 | or ANOVA | Levene | Pearl 1, 2 | Pearl 3 | or ANOVA | Levene |

A

| Patients with | # of cases | 165 | 99 | | | 58 | 46 | | |
|---|---|---|---|---|---|---|---|---|---|
| European | dosage | 34.5%/24.2%/ | 0%/52.5%/ | | | Placebo | Placebo | | |
| ancestry | (40/80/120/ | 41.2%/0% | 0%/47.5% | | | | | | |
| | 160 mg/d) | | | | | | | | |
| | % male | 66.10% | 61.60% | 0.465 | NA | 74.14% | 60.87% | 0.149 | NA |
| GWAS | # of SNPs | 1731293 | 1740224 | NA | NA | 1735614 | | NA | NA |
| data | included for | | | | | | | | |
| | meta-analyses | | | | | | | | |
| | λ GC | 1.01 | 1.00 | NA | NA | 1.00 | | NA | NA |
| Baseline | PANSS_TOT | 95.47(8.83) | 98.61(9.87) | 0.008 | 0.086 | 97.16(10.74) | 97.85(9.013) | 0.727 | 0.332 |
| Psychopathology | PANSS_POS | 19.96(3.14) | 19.23(3.01) | 0.064 | 0.652 | 20.28(3.24) | 18.87(2.35) | 0.015 | 0.033 |
| | PANSS_NEG | 22.58(4.56) | 23.32(4.56) | 0.198 | 0.888 | 22.52(4.39) | 23.76(3.73) | 0.128 | 0.604 |
| Δ change | PANSS_TOT | −15.88(16.97) | −21.95(18.15) | 0.007 | 0.846 | −14.67(19.05) | −8.54(19.03) | 0.106 | 0.814 |
| of | PANSS_POS | −5.15(4.93) | −5.71(4.35) | 0.35 | 0.141 | −4.50(4.96) | −2.50(4.91) | 0.044 | 0.916 |
| Psychopathology | PANSS_NEG | −3.26(4.82) | −3.85(4.88) | 0.34 | 0.896 | −3.00(4.80) | −2.04(4.18) | 0.291 | 0.322 |
| % change | PANSS_TOT | −16.55(17.79) | −22.47(19.05) | 0.011 | 0.732 | −15.11(19.49) | −8.71(18.92) | 0.095 | 0.774 |
| of | PANSS_POS | −25.80(24.91) | −30.04(22.51) | 0.166 | 0.243 | −22.30(25.10) | −13.47(25.61) | 0.083 | 0.769 |
| Psychopathology | PANSS_NEG | −13.44(22.75) | −15.66(22.26) | 0.441 | 0.845 | −13.42(19.84) | −8.12(17.01) | 0.155 | 0.191 |

B

| Patients with | # of cases | 124 | 34 | | | 49 | 12 | | |
|---|---|---|---|---|---|---|---|---|---|
| African | dosage | 44.35%/ | 0%/50%/ | | | Placebo | Placebo | | |
| ancestry | (40/80/120/ | 18.55%/ | 0%/50% | | | | | | |
| | 160 mg/d) | 37.10%/0% | | | | | | | |
| | % male | 75.81% | 76.47% | 0.936 | NA | 75.00% | 75.51% | 0.971 | NA |
| GWAS | # of SNPs | 1994688 | 1935575 | NA | NA | 1849733 | | NA | NA |
| data | included for | | | | | | | | |
| | meta-analyses | | | | | | | | |
| | λ GC | 1.00 | 1.00 | NA | NA | 1.01 | | NA | NA |
| Baseline | PANSS_TOT | 94.56(9.28) | 97.21(11.43) | 0.165 | 0.097 | 93.29(9.27) | 92.83(9.80) | 0.727 | 0.881 |
| Psychopathology | PANSS_POS | 20.50(2.90) | 21.65(2.78) | 0.041 | 0.911 | 20.16(3.33) | 21.08(2.61) | 0.015 | 0.377 |
| | PANSS_NEG | 22.08(4.44) | 23.21(5.27) | 0.211 | 0.139 | 22.31(4.31) | 22.33(4.68) | 0.128 | 0.985 |
| Δ change | PANSS_TOT | −15.88(16.97) | −21.95(18.15) | 0.007 | 0.846 | −12.78(18.47) | −5.00(17.56) | 0.106 | 0.192 |
| of | PANSS_POS | −5.15(4.93) | −5.71(4.35) | 0.35 | 0.141 | −3.92(4.57) | −3.00(4.55) | 0.044 | 0.535 |
| Psychopathology | PANSS_NEG | −3.26(4.82) | −3.85(4.88) | 0.34 | 0.896 | −3.00(4.81) | −1.25(4.59) | 0.291 | 0.260 |
| % change | PANSS_TOT | −16.55(17.79) | −22.47(19.05) | 0.011 | 0.732 | −13.64(18.77) | −5.11(18.91) | 0.095 | 0.164 |
| of | PANSS_POS | −25.80(24.91) | −30.04(22.51) | 0.166 | 0.243 | −18.94(21.53) | −15.25(22.86) | 0.083 | 0.600 |
| Psychopathology | PANSS_NEG | −13.44(22.75) | −15.66(22.26) | 0.441 | 0.845 | −12.98(21.24) | −4.96(20.21) | 0.155 | 0.242 |

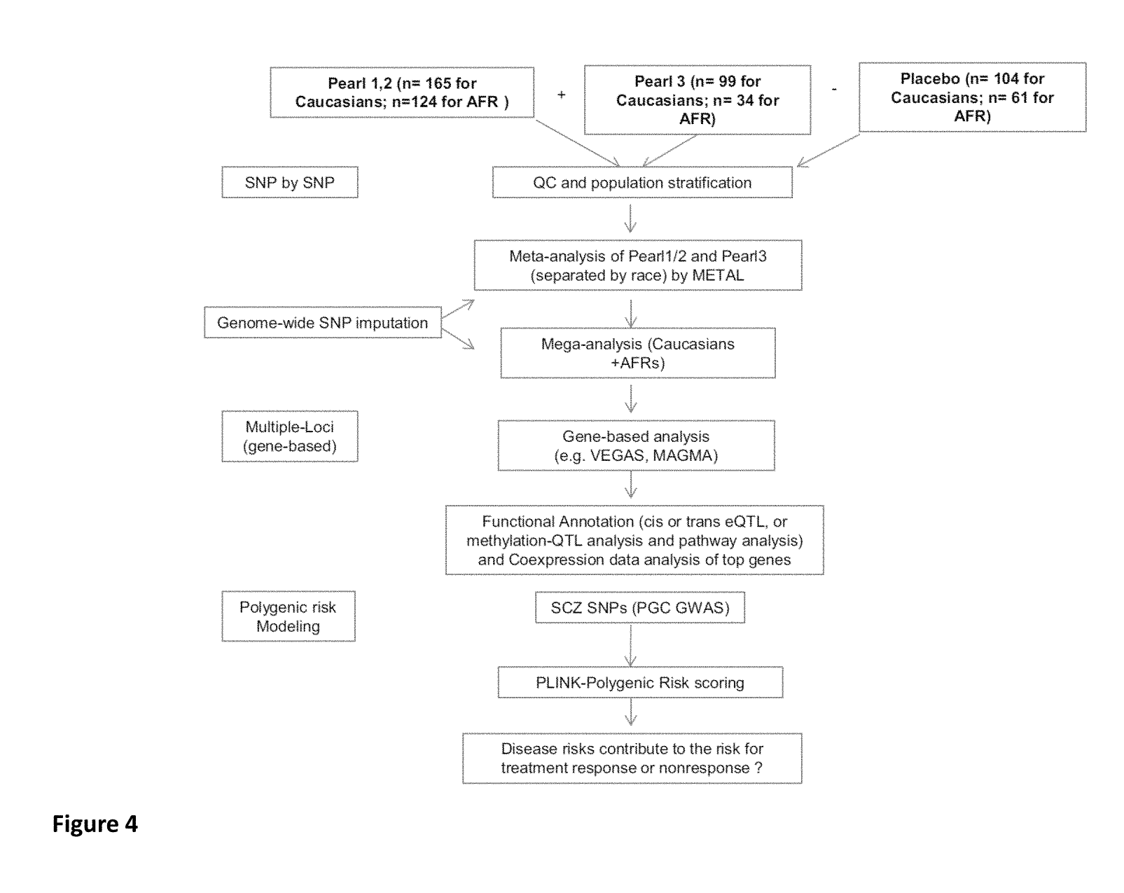

Pooled samples from PEARL 1 and 2 served as the discovery dataset because no significant differences were observed in the baseline psychopathological data (Li et al., 2018). PEARL 3 was considered an independent dataset due to a significant difference in PANSS-TOT_{Baseline}, inclusion of a higher dose of lurasidone (160 mg/d), and an unequal variance, compared to PEARL 1/2, in ΔPANSS-TOT_{LOCF6WK} by Leven test (Table 1). Samples from the corresponding placebo groups of all three trials were pooled together due to a relatively small sample size per trial and absence of significant differences in demographic and clinical data. Thus, three groups (PEARL 1+2, PEARL 3 and Placebo) per ethnicity were independently examined for quality control (QC), PCA for genetic architectures, and GWAS analyses before determining summary statistics at the individual SNP level. Both Kolmogorov-Smimov and Shapiro-Wilk tests indicated ΔPANSS-TOT_{LOCF6WK} followed a normal distribution with skewness and kurtosis indices between −1 and +1. Therefore, no subjects were excluded from the analysis. Illustration of the study and analytical pipeline is reported in FIG. 4.

Overview of the Method for Data Analysis.

Quality control for genotyping data was conducted to exclude SNPs with MAF<0.05, genotyping rate<0.95, and significant deviation from Hardy-Weinberg Equilibrium (HWE, p<0.0001). As shown in Table 1, covariates including gender, PANSS-TOT_{Baseline}, and dosage, were tested as a single covariate for ΔPANSS-TOT_{LOCF6WK}. Only dosage demonstrated a significant association with ΔPANSS-TOT_{LOCF6WK}. Therefore, a linear regression with an additive model for minor alleles, adjusted for dosage and genetic architecture (5major PCs), was utilized to test the association between the common genetic variants (MAF N 0.05) and ΔPANSS-TOT_{LOCF6WK}.

Genome-wide SNP imputation (IMPUTE2) and association testing (PLINK1.9) was performed in the genomic regions flanking the top loci with the genome-wide (p<5×10⁻⁸) or close to genome-wide (p<5×10⁻⁷) significance using the latest 1KG phase III integrated variant set (April 2014) as reference genome. Pre-phasing haplotypes was performed by SHAPEIT. For post-imputation SNP filtering, info value≥0.85 was considered as cutoff.

Meta-analyses within each ethnic group were accomplished using METAL (Willer et al., 2010) with fixed effect, inverse variance weighting, and genomic control. Mega-analysis among different ethnic groups was conducted based on the summary statistics of the meta-analyses.

For Gene-based multiple loci analysis, VEGAS (vegas2v2) (Mishra and Macgregor, 2015) and MAGMA (ver1.06) (de Leeuw et al., 2015), were conducted based on the p values from the meta-analysis within each ethnic groups.

Functional annotation was conducted including cis or trans eQTL (Braincloud, Braineac, and LIBD eQTL browser), coexpression network and pathway enrichment analysis of targets by SEEK (Zhu et al., 2015). We also used Cytoscape/ClusterOne (Nepusz et al., 2012) to visualize and cluster evidenced-based functional interaction of CTNNA2-coexpressed genes in dorsolateral prefrontal cortex (DLPFC) from Braincloud.

Finally, PLINK polygenic risk scoring (PRS) was used for polygenic risk modeling but only in the EUR patients. It was calculated based on the aggregated number of risk alleles identified from PGC GWAS after selection of SNPs at a step-wised p-level in the SNP-by-SNP association test, and then weighting the SNPs based on the Log of Odds Ratio from the PGC GWAS. The phenotypic variance of ΔPANSS was then predicted by linear regression analysis of PRS plus covariates, including PCs, dosage and study in the full model (Fanous et al., 2012). “Full SNP results” from the PGC “SCZ2 study” was acquired. Polygenic risk was calculated based on the prune-in sets of SNPs after LD pruning of PEARL 1/2/3 dataset by PLINK command (−indep 50 5 1.5), as inclusion of correlated SNPs that do not contain independent signals can significantly reduce the predictive performance of models (Dudbridge, 2013, and data not shown).

Results

Top Hits with Genome-Wide or Close to Genome-Side Significant Association.

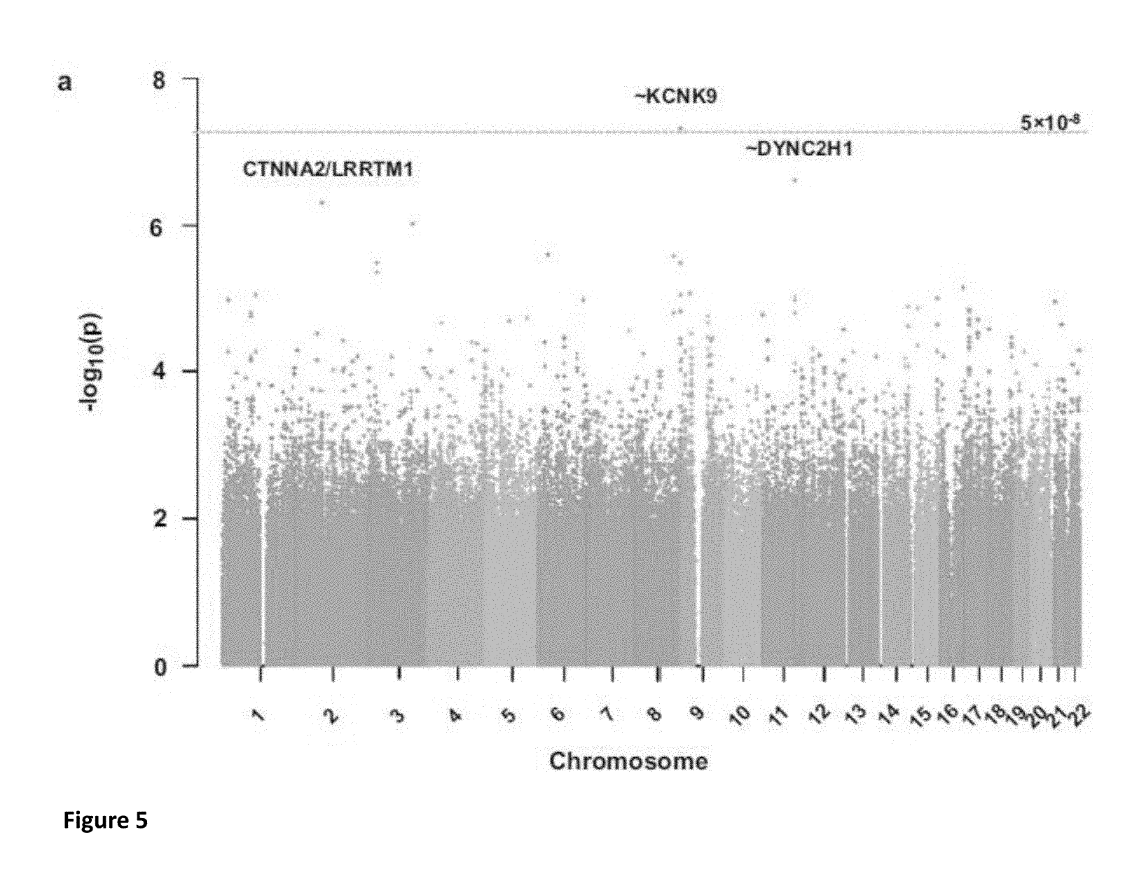

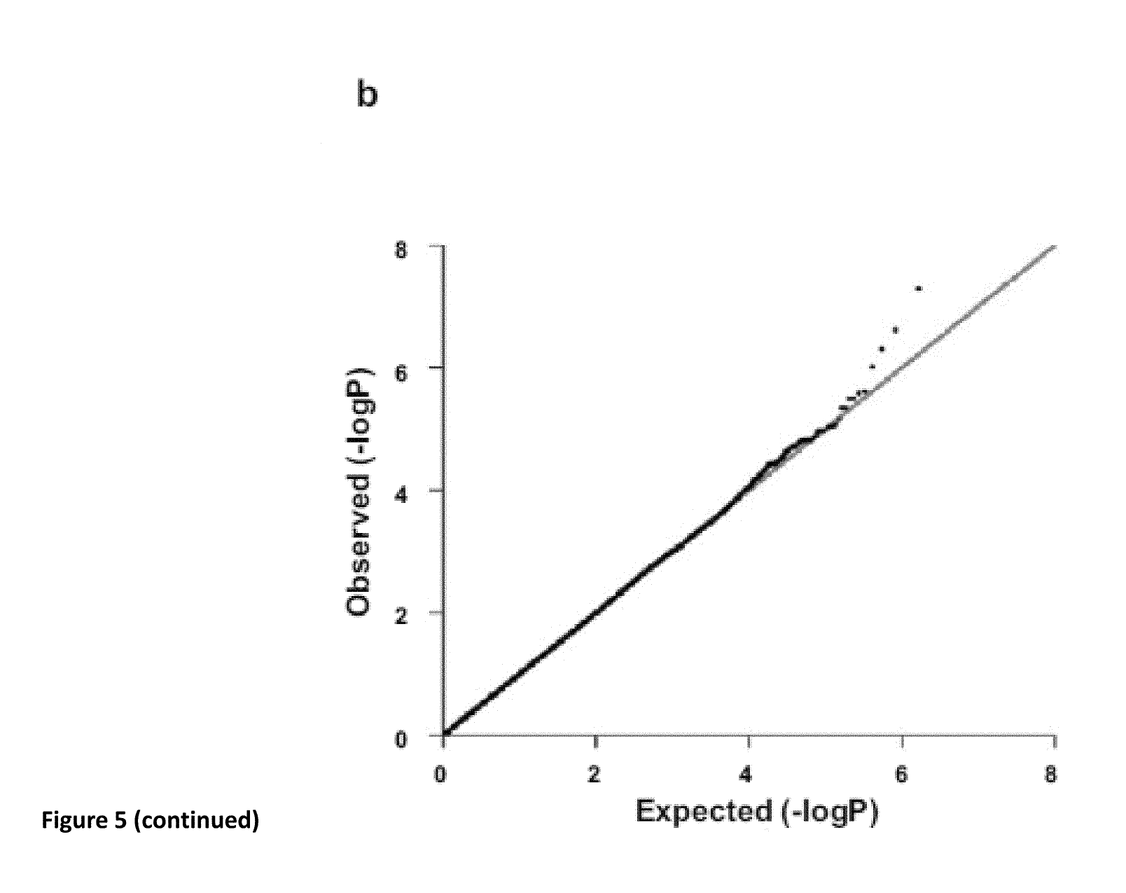

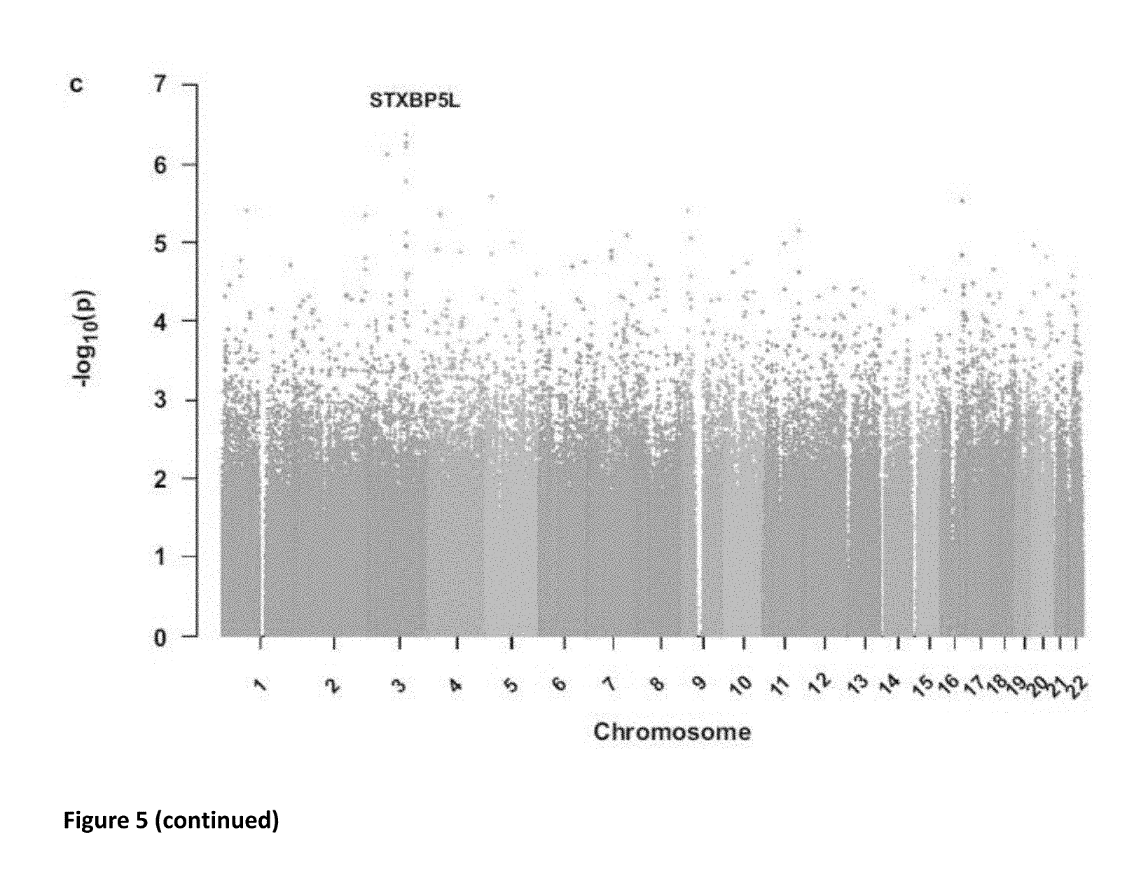

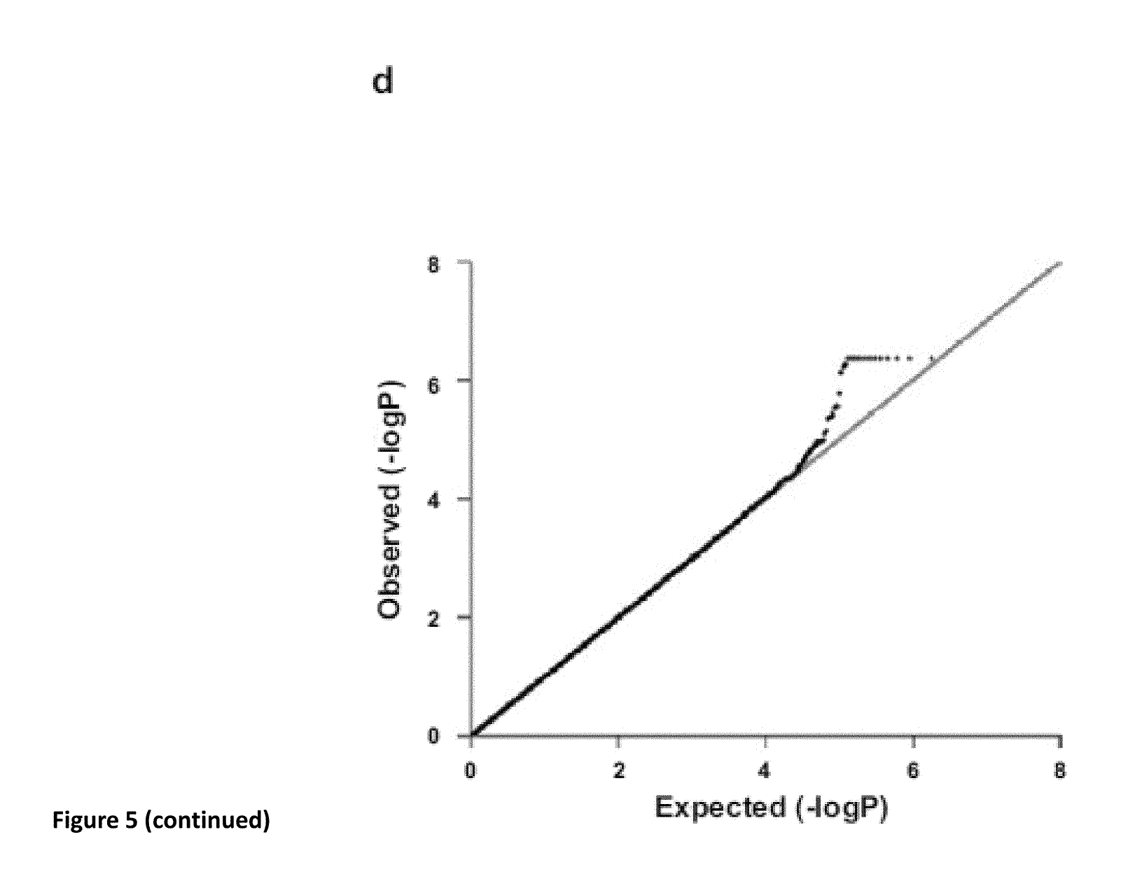

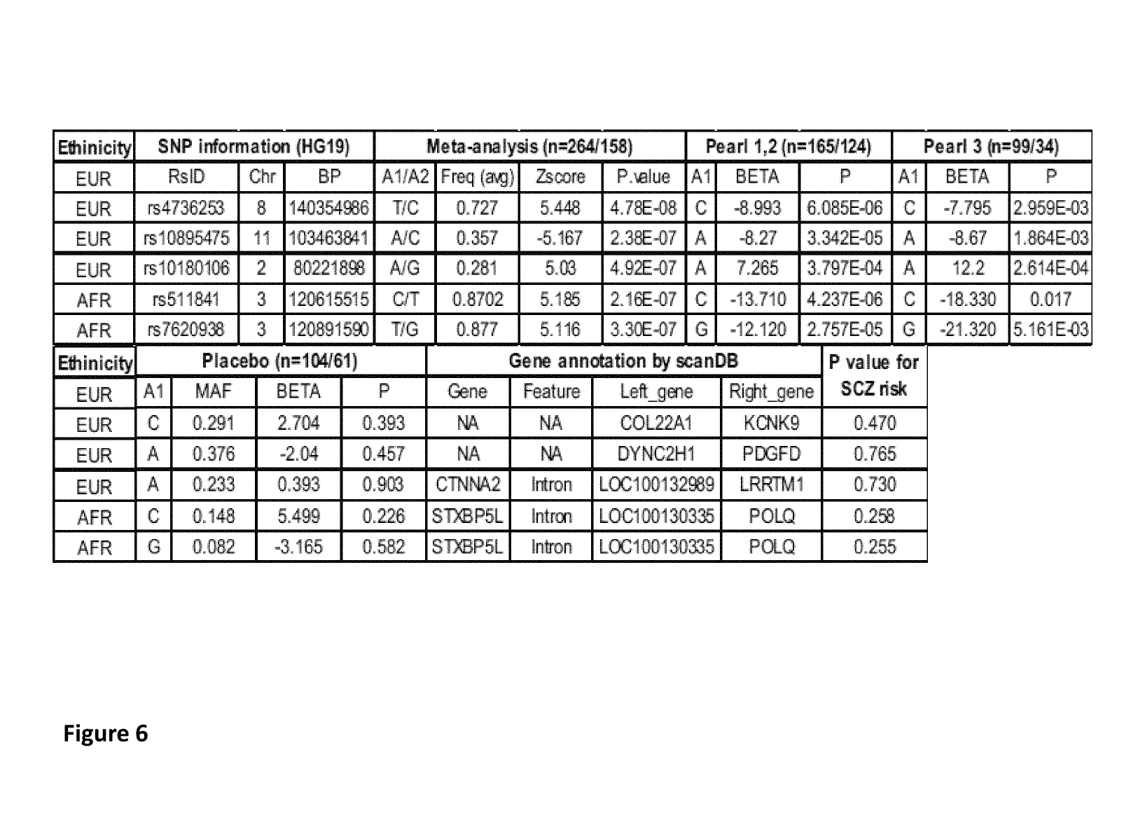

Top hits with genome-wide or close to genome-wide significant association are indicated in the Manhattan plots for EUR and AFR (FIGS. 5A and 5C).

Associations with clinical response in the EUR patients rs4736253, a genetic locus at 8q24, reached GWS (p=4.78×10⁻⁸, data not shown) in the EUR patients, followed by rs10895475 located near DYNC2H1 (p=2.38×10⁻⁷ (data not shown)) and rs10180106, located at CTNNA2 (p=4.92×10⁻⁷, (data not shown)). The closest gene to rs4736253 is KCNK9 (˜360 kb away from the Transcription Start Site), which encodes K_{2P}9.1, a member of the two pore-domain potassium channel (K_{2P}) subfamily. This SNP showed partial LD with rs7017126 (r²=0.498 and D′=0.795) or its tag SNP rs3857923 (r²=0.450 and D′=0.717), a top marker previously identified in the PEARL 1/2 sample (Li et al., 2018) at this locus (data not shown).

According to Braineac, an eQTL database from EUR subjects, rs4736253 has a nominal effect on the expression of KCNK9, strongest in temporal cortex (p=0.001) (data not shown). This locus was previously identified in the PEARL 1/2 sample as one of three shared genomic regions (PTPRD, MAGI1, and COL22A1/KCNK9), associated with response to lurasidone in both EUR and AFR patients (Li et al., 2018). The minor allele of rs13270196, a proxy for rs9644441 located near KCNK9, was the top signal (p=2.668×n=124) for PEARL 1/2 at this locus and showed the same direction for 13 in PEARL 3, but was not significant (p=0.821, n=34) (data not shown). rs4736253 identified in EUR was not in LD with SNPs, rs13270196 or rs964441, identified in AFR. COL22A1 is another gene close to rs4736253 (data now shown). Its expression in various brain tissues (pituitary not included) is very low according to GTX and Allen Brain Atlas.

According to Braincloud, rs10180106 significantly affected the expression of CTNNA2 in the EUR patients (n=75, p=9.579×10⁻⁶), but not in the AFR patients (n=91, p>0.05), when a probe ID, 5492, flanking 3′UTR of CTNNA2 was selected. Homozygous AFR carriers showed the lowest expression of CTNNA2 compared with AG (p=2.847×10⁻⁶) or GG (p=2.196×10⁻⁶) carriers (data not shown). Interestingly, homozygous AFR patients showed less response to lurasidone than those with AG (p=0.0038) or GG (0.00014) genotypes in PEARL 1/2 (data not shown); Patients with heterozygous AG showed poor response compared to those with GG (p=0.074 for PEARL 1/2 and p=0.0034 for PEARL 3, (data not shown)). This impact on gene expression was also confirmed by Braineac (data not shown), using different probes, and in substantial nigra (p=0.0055), the origin of the DA neurons that project to the dorsal striatum. A nearby SNP, rs13394481, 4884 bp upstream of rs10180106, was the top signal (p=7.52×10⁻⁴) for AFR patients within the coding region±50 kb of CTNNA2 (data not shown). This SNP was not in LD with rs10180106 in either EUR (r²/D′=0.225/0.652) or AFR patients (r²/D′=0.054/0.330). A further conditional linear regression and eQTL analysis showed that GT-CC and GT-CT carriers of the rs13394481-rs1541947 genotype combination showed lower expression than other genotype combinations in AFR (data not shown). Therefore, both EUR and AFR patients identified as having a lower expression of CTNNA2, had diminished response to lurasidone. Through co-localization and a correlation analysis of eQTL (DLPFC, Braincloud) and GWAS signals (lurasidone pharmacogenomics) at the genomic region of CTNNA2 (Chr2: 79.50-80.73 MB), we confirmed that rs10180106 and rs13394481 were the shared top genetic risks for both traits in EUR or AFR patients respectively. This identifies CTNNA2 as a robust predictor of response to lurasidone, independent of ethnicity. Its significance is enhanced by the co-localization of the strongest eQTL and phenotypic association signals in this region as reported in other studies (Giambartolomei et al., 2014), suggesting it is a causal gene and the DLPFC is an area in which the CTNNA2 genetic polymorphism may have its greatest effect.

It is noteworthy that rs10180106, rs13394481, and rs1541947, are located in a genomic region which bi-directionally expresses CTNNA2 and LRRTM1. rs10180106, rs13394481 and rs1541947 showed no effect on the expression of LRRTM1 (data not show) in the EUR or AFR patients.

Associations with Clinical Response in the AFR Patients.

Multiple SNPs at STXBP5L (p=4.33×10⁻⁷) were the top markers for AFR (n=124/34 for PEARL 1+2/3). Through regional SNP imputation, rs511841 near 5′UTR of STXBP5L showed the strongest association with p=2.63×10⁻⁷ (data not shown). All other top signals came from the entire coding region of STXBP5L. 6 or 17 clumps formed after LD-based pruning with the clumping parameters set as r²=0.5/p1=0.0001 or r²=0.5/p1=0.01, suggesting there were several independent signals associated with clinical response at STXBP5L region. Therefore, a gene-based test, as shown later, is likely to perform better, based on multiple independent signals rather than a single signal identified from one gene. There was no evidence from Braincloud, an eQTL database based on the tissue collected from DLPFC of subjects mainly with EUR or AFR ancestries, to support the conclusion that those SNPs significantly impact expression of STXBP5L in brains of AFR subjects.

Comparison of Results from PEARL 3 with Those of PEARL 1/2.

Here, we only selected SNPs with p<1.00×10⁻⁴, previously reported to be top tier among those associated with ΔPANSS-TOT_{LOCF6WK}. The lurasidone samples genotyped in this study for PEARL 1/2 represent 96.5% (EUR) and 94.7% (AFR) of sample previously reported with Affymetrix 6.0 SNP array (Li et al., 2018). Both array platforms are designed to cover>85% of common variants of the genome for EUR. Through a proxy SNP search by SNAP (r²>0.8, distance limit<500 kb, CEU or YRI) using 1KG Pilot 1 data as the source, we found tagged SNPs in LD with 41 (EUR) or 40 (YRI) SNPs previously reported (data not shown). The direction of β in PEARL 3 for 24/41 (EUR) and 22/40 (AFR) SNPs was consistent with that in PEARL 1,2. 6/24 (EUR) or 5/22 (AFR) SNPs showed, at least, a trend of significant association in PEARL 3 (p<0.15). This included NRG1 (EUR, p=0.017 for rs16879886), MAGI1 (EUR, p=0.024 for rs11922361), a cell/synaptic scaffolding protein and PTPRD (EUR, p=0.132 for rs2093483), a synaptic adhesion molecular. According to LIBD eQTL browser for DLPFC, rs16879886 is an eQTL for NRG1 (β=−0.100, p=9.175×10⁻⁵, pFDR=0.008). All of the above SNPs showed no association to response in the corresponding placebo groups (n=104/61 for EUR/AFR).

Gene-Based Association Testing Using Summary Statistics from the Meta-Analysis Identified a GWS Gene, STXBP5L.

According to VEGAS and MAGMA, STXBP5L was the top gene ranked by p value which was GWS (p_{top10%}=1.00×10⁻⁶ with 1.00×10⁻⁶ simulations for VEGAS; p_{top10%}=1.67×10⁻⁵ with 6.6×10⁻⁵ simulations for MAGMA) in gene-based association testing for the AFR samples. No genes reached GWS in the EUR samples.

Trans-Ethnic Mega-Analysis.

The result of trans-ethnic mega-analysis are given in Table 4.

TABLE 4
Summary of trans-ethnic meg-analysis. Top variants were listed with
LD-based clumping (-clump-r² and -clubp-p1 2 × 10⁻⁵).

| SNP information (HG19) | | | | Mega-analysis | | Meta-analysis from EUR | | |
|---|---|---|---|---|---|---|---|---|
| RsID | Chr | BP | A1/A2 | Zscore | p_value | Freq_A1 | Zscore | p_value |
| rs524045 | 1 | 93,490,026 | A/G | 4.918 | 8.73E−07 | 0.1151 | 4.3 | 1.71E−05 |
| rs642516 | 11 | 79,000,128 | A/G | 4.803 | 1.57E−06 | 0.6926 | 3.252 | 0.001145 |
| rs4507566 | 6 | 66,262,055 | A/G | −4.756 | 1.98E−06 | 0.5584 | −3.44 | 0.000583 |
| rs10180106 | 2 | 80,221,897 | A/G | 4.719 | 2.37E−06 | 0.2813 | 5.03 | 4.92E−07 |
| rs4736253 | 8 | 140,354,985 | T/C | 4.718 | 2.38E−06 | 0.7266 | 5.448 | 4.78E−08 |
| rs60354593 | 4 | 149,594,421 | T/G | −4.557 | 5.19E−06 | 0.1435 | −3.984 | 6.77E−05 |
| rs26193 | 5 | 35,363,908 | A/G | 4.475 | 7.64E−06 | 0.3048 | 2.551 | 0.01074 |
| rs12857574 | 13 | 107,061,427 | A/C | 4.441 | 8.95E−06 | 0.3397 | 4.004 | 6.23E−05 |
| rs4767683 | 12 | 118,958,607 | A/G | 4.403 | 1.07E−05 | 0.1208 | 3.767 | 0.000165 |
| rs4968574 | 17 | 59,650,882 | T/C | −4.342 | 1.41E−05 | 0.7942 | −3.511 | 0.000446 |
| rs4733373 | 8 | 32,605,582 | A/G | −4.327 | 1.51E−05 | 0.8783 | −3.798 | 0.000146 |
| rs4532282 | 4 | 190,356,060 | T/C | −4.309 | 1.64E−05 | 0.7719 | −3.743 | 0.000182 |
| rs6142655 | 20 | 60,111,742 | A/G | −4.279 | 1.88E−05 | 0.8939 | −3.031 | 0.002437 |

| Meta-analysis from AFR | | | Gene annotation (scandb.org) | | | |
|---|---|---|---|---|---|---|
| Freq_A1 | Zscore | p_value | Gene | Feature | Left_gene | Right_gene |
| 0.1234 | 2.48 | 0.01314 | NA | NA | LOC100133115 | MTF2 |
| 0.7215 | 3.645 | 0.000268 | NA | NA | ODZ4 | LOC646112 |
| 0.5063 | −3.326 | 0.000882 | EGFL11 | Intron | RP11-74E24.2 | LOC442229 |
| 0.1784 | 1.206 | 0.228 | CTNNA2 | Intron | LOC100132989 | LRRTM1 |
| 0.8038 | 0.761 | 0.4466 | NA | NA | COL22A1 | KCNK9 |
| 0.3386 | −2.297 | 0.02161 | NA | NA | ASSP8 | LOC100130396 |
| 0.5411 | 4.013 | 5.99E−05 | NA | NA | PRLR | SPEF2 |
| 0.269 | 2.082 | 0.03731 | NA | NA | LOC728192 | LOC41604 |
| 0.1899 | 2.326 | 0.02001 | NA | NA | SUDS3 | KIAA1853 |
| 0.8829 | −2.557 | 0.01056 | NA | NA | TBX4 | NACA2 |
| 0.7089 | −2.165 | 0.03041 | NRG1 | Intron | LOC100127894 | MST131 |
| 0.9051 | −2.204 | 0.02752 | NA | NA | LOC285442 | HSP90AA4P |
| 0.6393 | −3.075 | 0.002107 | CDH4 | Intron | MTCO2L | RP11-429E11.3 |

No GWS markers were identified. The top variant, rs524045 (p=8.73×10⁻⁷) is located at the intergenic region between CCDC18 and MTF2 and is an eQTL for CCDC18 (p=4.117×10⁻⁷, FDR=6.617×10⁻⁵ from LIBD eQTL browser for DLPFC). rs642516 (p=1.57×10⁻⁶) near ODZ is ranked the 2nd. This SNP showed moderate effect on the gene expression of ODZ at Medulla (p=6.90×10⁻³, Braineac). Others in this category include SNPs at EYS, CDH4, CTNNA2, and NRG1. It is interesting that SNPs (rs4733373, rs4733372, rs16879886) in LD at NRG1 were initially discovered in PEARL 1/2 EUR patients (Li et al., 2018) and demonstrated a trend for significant association with response in PEARL 3 EUR (p=0.017 for rs4733373) and AFR (pₘₑₜₐ=0.03 for rs4733373) as they did for PEARL 1/2.

Polygenic SCZ Risk from PGC GWAS has a Limited Power to Contribute to the Treatment Response.

We next examined which SNPs identified by PGC GWAS as SCZ risk factors, were significantly associated with lurasidone response. We used a two-stage approach: 1) determination of the association between PGC GWAS SNPs and treatment response at the individual SNP level; and 2) determination of the association at the polygenic level by creating polygenic risk scores from SNPs with 17 consecutive levels of significant association with SCZ. 2145 SNPs with genome-wide significant association (p<5×10⁻⁸) with SCZ risk were interrogated for their association with ΔPANSSTOT_{LOCF6WK}. 78 of 108 SCZ risk loci were available in the PEARL datasets. For the initial SNP-by-SNP association test, those SNPs with uncorrected p value<0.03 were associated with ΔPANSSTOT_{LOCF6WX} but none survived Bonferroni correction (data not shown). The alleles, from those SNPs annotated for CACNA1C (rs2239037, pₘₑₜₐ=0.005; rs2007044, pₘₑₜₐ=0.041), TCF4 (rs9636107, pₘₑₜₐ=0.011), SNAP91 (rs971215, pₘₑₜₐ=0.019), ‘12q24’, ‘17p11’ which showed increased risk for SCZ, were associated with a poor response to lurasidone. Loci at SNX19, ‘3p21’, ‘1q21’, which showed increased risk for SCZ, were associated with a better response to lurasidone.

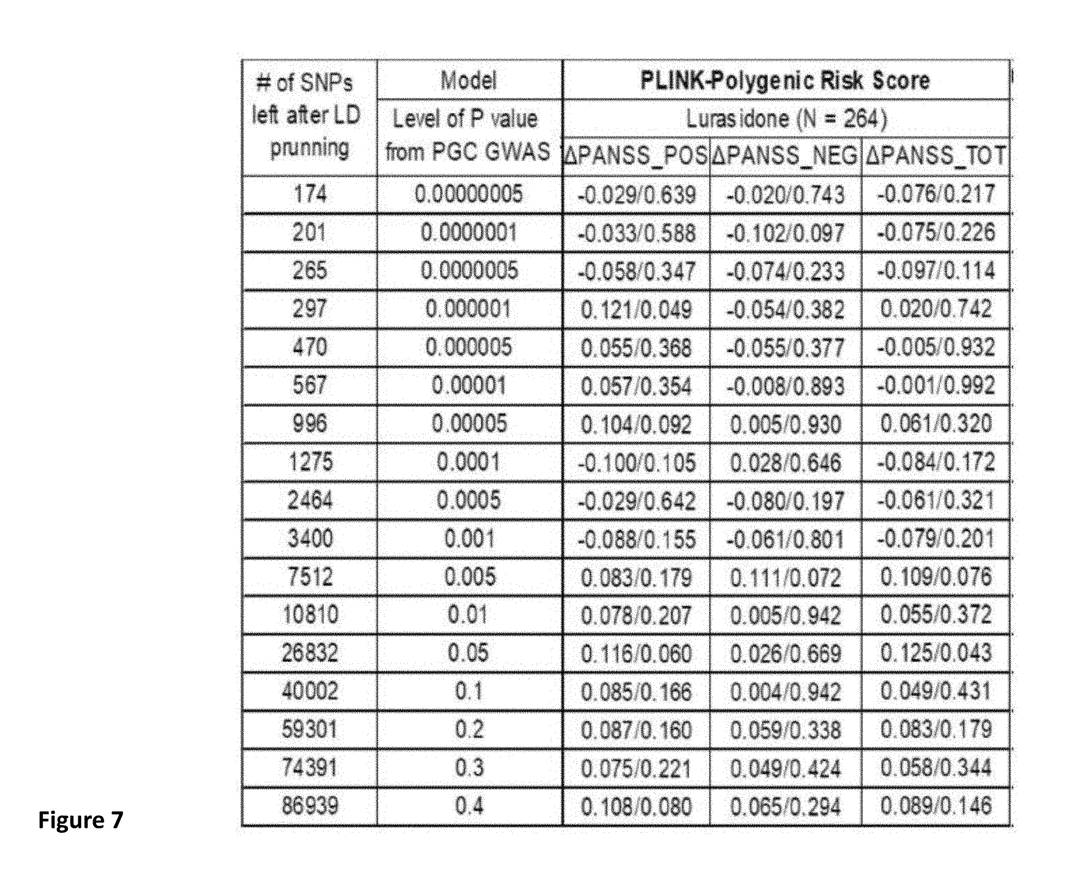

Although we observed a trend for an association between polygenic risk score (FIG. 7) and treatment response with increased overall SCZ risk having poor response to lurasidone (β>0), particularly in ΔPANSS-POS, this association was not statistically significant, suggesting that the common variants for SCZ risk so far identified by PGC GWAS, collectively, have limited power to predict response to lurasidone.

Discussion

The goal of this study was to determine whether we could validate or not the results of our previous pharmacogenomics study of response to lurasidone by including additional samples in a meta-analysis. The additional samples included in this meta-analysis enabled this to be the second largest study to identify predictors of antipsychotic response to a single APD. With the additional samples, we were able to identify SNPs from two genes linked to SCZ with GWS at the SNP or gene level in two ethnic groups. rs4736253, a genetic locus at 8q24, close to KCNK9 (p=4.78×10⁻⁸), was the top marker in EUR subjects, followed by rs10895475 near DYNC2H1 (p=2.38×10⁻⁷), and rs10180106 at CTNNA2 (p=4.92×10⁻⁷). SNPs at STXBP5L (p=2.63×10⁻⁷) were the top markers for AFR subjects. STXBP5L was the top gene ranked by p value with a GWS (p=1.00×10⁻⁶ by VEGAS and 1.67×10⁻⁵ by MAGMA) in the gene-based analyses. A number of the findings from PEARL 1/2 were supported as or more strongly with the additional data, suggesting that the findings are robust and may have clinical and theoretical significance.

Identifying the causal variants or causal genes can be made by confirmation of association signals through replication in independent samples from the same or different populations (Ioannidis et al., 2009). We also identified nearby SNPs from KCNK9 and CTNNA2 which showed independent predictive value in AFR subjects. SNPs identified from EUR or AFR subjects within CTNNA2 showed significant impact on gene expression in the corresponding ethnic group. Patients predicted to have lower expression level of CTNNA2, have diminished response to lurasidone compared to those with higher expression of CTNNA2, suggesting development of drugs which increase expression of CTNNA2 or which enhance its activity is a potential strategy for treatment of SCZ and related disorders. We also found shared genetic risk factors across ethnicities for treatment response. Although the trans-ethnic mega-analysis led to no GWS markers, SNPs from several genes with biological relevance to synaptic biology and/or SCZ were ranked at the top of our list of biomarkers for lurasidone response (TABLE 2). These include ODZ, EYS, NRG1, and CDH4. Of these NRG1 and CDH4 have a long pedigree of interest in schizophrenia research (data not shown). Together, these findings provide additional support that the mechanism of action of APDs is linked to pathogenesis of SCZ. This suggests that AAPDs treatment may affect SCZ disease progression, including relapse. If so, lurasidone and other AAPDs, may modify the course of the disease if the genes they interact with are of sufficient importance to influence the effects of genes or G (genes)×E (environmental factors) interaction.

Effect of Additional Samples on Prior Report of PEARL 1/2 Biomarkers.

The association of some gene markers previously reported in the pharmacogenomic study of PEARL 1/2 remained significant at the same level or were even stronger after the inclusion of PEARL 3 data in the meta-analysis. Those include PTPRD (EUR), a synaptic adhesion molecular, NRG1 (EUR/AFR), and MAGI1 (EUR), a cell/synaptic scaffolding protein. The NRG1-ERBB4 pathway has a profound impact on activity-dependent synaptic spine formation, connectivity in brain and neuro-muscular junction, and drug efficacy of AAPDs based on numerous preclinical (Agarwal et al., 2014; Zhang et al., 2016) and pharmacogenetics studies (Zai et al., 2017). Although many identified synapse-related genes such as STXBP5L, NRG1, PTPRD, CTNNA2, and SNAP91 were associated with treatment response to lurasidone and also are reported targets of RBFOX1 (Linden et al., 2008; Weyn-Vanhentenryck et al., 2014), previously identified SNPs from RBFOX1 were not validated in the meta-analyses.

The Biological Relevance of Top Genes to SCZ

KCNK9.

KCNK9, also known as TASK3, is a member of the two pore-domain potassium channel (K2P) subfamily (Kim et al., 2000), which form leak conductances that regulate neuronal excitability. KCNK9 has a brain-specific gene expression (GTEx) and has been shown to have a role in neurodevelopment and cognition (Barel et al., 2008; Berg et al., 2004; Goldstein et al., 2005; Kim et al., 2000; Linden et al., 2008; Pang et al., 2009). KCNK9 knockouts show upregulation of GABAA receptors (Linden et al., 2008) which might be related to the abnormalities in GABA function in schizophrenia (Bates et al., 2014; Chung et al., 2016).

CTNNA2.

The αN-catenin (CTNNA2) gene is a key regulator of synaptic spine turnover, formations and stability of synaptic contacts (Abe et al., 2004). CTNNA2 has been recognized as a SCZ risk gene based upon genetic linkage, gene expression and coexpression network (data not shown), in vitro functional studies, and in vivo knockout studies (Abe et al., 2004; Mexal et al., 2005; Park et al., 2002; Smith et al., 2005; Terracciano et al., 2011). Under or overexpression of αN-catenin produced abnormalities in spines would be expected to have adverse effects on the ability of AAPDs to normalize deficits in positive and negative symptoms and cognitive impairment. Coexpression network analysis by SEEK showed that the top enriched pathways from the top 200 genes co-expressed with CTNNA2 in prefrontal cortex are neuron differentiation (q value-0.039), cell projection organization (q value=0.0431), and negative regulation of microtubule depolymerization (q value=0.00426), further confirming its functional impact on spine formation and stabilization.

STXBP5L.

Converging evidence indicates STXBP5L, also known as tomosyn-2, may play an important role in vesicle trafficking and exocytosis in presynaptic neurons and neuromuscular junctions during early childhood development (Geerts et al., 2015; Kumar et al., 2015). Coexpression network analysis by SEEK showed that STXBP5L is significantly co-expressed with SNARE, multiple GABAA and GABAB subunits and GRIN2A in multiple brain regions. The top enriched pathways for STXBP5L from the 200 most co-expressed genes includes synaptic transmission (q=0.00382) and post Golgi vesicle mediated transport (q=0.00768).

It is of interest that all three of our top hits in the meta-analysis related to GABA, actin cytoskeleton, and synaptic morphology and function.

Polygenic Risk Modeling Using SCZ Risk SNPs as Candidates.

PLINK-PRS weighted the individual SNPs by including the direction and effect size of the risk alleles for risk for SCZ. Our goal was to determine whether increased load of genetic risk for SCZ affects treatment response. However, PRS analysis did not find a significant association between PRS and ΔPANSS-TOT_{LOCF6WK} or its two subscales, suggesting an overall increase in the genetic risk for SCZ did not predict response to lurasidone (data not shown). Previous studies (Li and Meltzer, 2014; Wimberley et al., 2017) and ours preliminary data (Li J, et al. unpublished) also showed no significant association between the PRS and Treatment-Resistant Schizophrenia. Therefore, this study does not provide support for the use of PRS derived from SCZ risk to predict response at the individual patient level (Wimberley et al., 2017).

TCF4 (Page et al., 2018), CACNA1C (Cosgrove et al., 2017), SNAP91 could be potential drug targets for the development of novel treatments for SCZ. A recent comparative genomic study showed that schizophrenia risk genes from PGC GWAS and historical candidate genes are differentially expressed following chronic haloperidol exposure (Kim et al., 2018). In this study, we found alleles from SNPs at TCF4, SNAP91 and CACNA1C, with increased risk for SCZ, had a poor response to lurasidone (data not shown).

Power Analyses.

A limitation of this study is exclusion of rare variants which may presumably have bigger effect sizes for prediction of lurasidone response. Due to the small sample size of this study, the power to detect the effect of these rare variants is low. Given the main effect of β_{G} (˜8 for top markers in EUR of Pearl 1/2 and Pearl 3), a type 1 error rate of 1×10⁻⁴ for nominal significance with a two sided test, on the continuous trait with mean±SD of ΔPANSS-T as −17±17, we conducted a power test using QUANTO. Our sample size of 264 EUR had >83% power to identify a significant association when MAF was equal to 0.26, and N>92% when MAF was equal to 0.36. The sample size of 158 AFR with effect size of ˜13 for the top markers in AFR had >80% power to identify a significant association when MAF was equal to 0.13.

It is to be hoped that a larger pharmacogenomic study of lurasidone would enable inclusion of rare variants in the polygenic model. If response genes to AAPDs are shared, then pooling the data from the lurasidone pivotal trials with those of similar drugs, e.g. risperidone, olanzapine, ziprasidone, quetiapine, might provide shared markers as well as those which are unique to each drug. The results reported here justify collection of DNA with appropriate consent from patients in future clinical APD trials. Combination of data from multiple GWAS not only improves the power of the association but also make possible identification of previously undetected associated loci. This could lead to the discovery of additional variants (Ioannidis et al., 2009). Here we showed that, CTNNA2 SNP was not reported as a top tier (p<10⁻⁴) marker in the PEARL 1/2. After the meta-analysis of data from all three trials, this SNP was ranked highly with close to GWS. More sophisticated machine learning models may provide a better simulation of G×G and G×E in a non-linear polygenic environment than linear models such as PLINK-PRS (Chatterjee et al., 2016; Cordell, 2009).

Conclusion and Limitation

In conclusion, this study provides some insight into the mechanism of action of lurasidone and most likely other AAPDs, suggesting that previously identified genes related to synaptic biology and schizophrenia risk genes may be related to improvement in overall psychopathology as measured by the PANSS in SCZ patients. Through an integration of GWASs from multiple clinical trials with a similar design, we have demonstrated it is possible to identify functionally relevant biomarkers and/or potential drug targets for APDs even with a relatively small sample. Although lurasidone has been found to improve cognitive impairment associated with schizophrenia (CIAS) in some clinical trials and in NMDAR antagonist models of CIAS, cognitive test results were available only for the third trial, Pearl 3, and insufficient to provide the power for a meaningful examination of genetic predictors of improvement in cognitive domains by lurasidone.

Supplementary data can be found online at doi.org/10.1016/j.schres.2018.04.006. which accompanies Li et al., “Identifying the genetic risk factors for treatment response to lurasidone by genome-wide association study: A meta-analysis of samples from three independent clinical trials,” Schizophr. Res. 2018 September; 198: 203-213, epub May 2, 2018.

### REFERENCES

Abe, K., Chisaka, O., Van Roy, F., Takeichi, M., 2004. Stability of dendritic spines and synaptic contacts is controlled by alpha N-catenin. Nat. Neurosci. 7 (4), 357-363.
Agarwal, A., Zhang, M., Trembak-Duff, I., Unterbamscheidt, T., Radyushkin, K., Dibaj, P., Martins de Souza, D., Boretius, S., Brzozka, M. M., Steffens, H., Berning, S., Teng, Z., Gummert, M. N., Tantra, M., Guest, P. C., Willig, K. I., Frahm, J., Hell, S. W., Bahn, S., Rossner, M. J., Nave, K. A., Ehrenreich, H., Zhang, W., Schwab, M. H., 2014. Dysregulated expression of neuregulin-1 by cortical pyramidal neurons disrupts synaptic plasticity. Cell Rep. 8 (4), 1130-1145.
Arranz, M. J., de Leon, J., 2007. Pharmacogenetics and pharmacogenomics of schizophrenia: a review of last decade of research. Mol. Psychiatry 12 (8), 707-747.
Barel, O., Shalev, S. A., Ofir, R., Cohen, A., Zlotogora, J., Shorer, Z., Mazor, G., Finer, G., Khateeb, S., Zilberberg, N., 2008. Maternally inherited Birk Barel mental retardation dysmorphism syndrome caused by a mutation in the genomically imprinted potassium channel KCNK9. Am. J. Hum. Genet. 83 (2), 193-199.
Bates, R. C., Stith, B. J., Stevens, K. E., Adams, C. E., 2014. Reduced CHRNA7 expression in C3H mice is associated with increases in hippocampal parvalbumin and glutamate decarboxylase-67 (GAD67) as well as altered levels of GABA(A) receptor subunits. Neuroscience 273, 52-64.
Berg, A. R, Talley, E. M., Manger, J. P., Bayliss, D. A., 2004. Motoneurons express heteromeric TWIK-related acid-sensitive K+(TASK) channels containing TASK-1 (KCNK3) and TASK-3 (KCNK9) subunits. The Journal of Neuroscience: The Official Journal of the Society for Neuroscience 24 (30), 6693-6702.
Charlab, R., Zhang, L, 2013. Pharmacogenomics: historical perspective and current status. Pharmacogenomics. Springer, pp. 3-22.
Chatterjee, N., Shi, J., Garcia-Closas, M., 2016. Developing and evaluating polygenic risk prediction models for stratified disease prevention. Nat. Rev. Genet. 17 (7), 392-406.
Chung, D. W., Fish, K. N., Lewis, D. A., 2016. Pathological basis for deficient excitatory drive to cortical parvalbumin interneurons in schizophrenia. Am. J. Psychiatry 173 (11), 1131-1139.
Cordell, H. J., 2009. Detecting gene-gene interactions that underlie human diseases. Nat. Rev. Genet. 10 (6), 392-404.
Cosgrove, D., Mothersill, O., Kendall, K., Konte, B., Harold, D., Giegling, I., Hartmann, A., Richards, A., Mantripragada, K., Owen, Mi., O'Donovan, M. C., Gill, M., Rujescu, D., Walters, J., Corvin, A., Morris, D. W., Donohoe, G., 2017. Cognitive characterization of schizophrenia risk variants involved in synaptic transmission: evidence of CACNA1C's role in working memory. Neuropsychopharmacology: Official Publication of the American College of Neuropsychopharmacology 42 (13), 2612-2622.
Dudbridge, F., 2013. Power and predictive accuracy of polygenic risk scores. PLoS Genet. 9 (3), e1003348.
Fanous, A. H., Zhou, B., Aggen, S. H., Bergen, S. E., Amdur, R. L., Duan, J., Sanders, A. R., Shi, J., Mowry, B. J., Olincy, A., Amin, F., Cloninger, C. R., Silverman, J. M., Buccola, N. G., Byerley, W. F., Black, D. W., Freedman, R., Dudbridge, F., Holmans, P. A., Ripke, S., Gejman, P. V., Kendler, K S., Levinson, D. F., 2012. Genome-wide association study of clinical dimensions of schizophrenia: polygenic effect on disorganized symptoms. Am. J. Psychiatry 169 (12), 1309-1317.
Geerts, C. J., Plomp, J. J., Koopmans, B., Loos, M., van der Pijl, E. M., van der Valk, M. A., Verhage, M., Groffen, A. J., 2015. Tomosyn-2 is required for normal motor performance in mice and sustains neurotransmission at motor endplates. Brain Struct. Funct. 220 (4), 1971-1982.
Giambartolomei, C., Vukcevic, D., Schadt, E. E., Franke, L., Hingorani, A. D., Wallace, C., Plagnol, V., 2014. Bayesian test for colocalisation between pairs of genetic association studies using summary statistics. PLoS Genet. 10 (5), e1004383.
Goldstein, S. A., Bayliss, D. A., Kim, D., Lesage, F., Plant, L. D., Rajan, S., 2005. International Union of Pharmacology. L V. Nomenclature and molecular relationships of two-P potassium channels. Pharmacol. Rev. 57 (4), 527-540.
Ioannidis, J. P., Thomas, G., Daly, Mi., 2009. Validating, augmenting and refining genomewide association signals. Nat. Rev. Genet. 10 (5), 318-329.
Ishibashi, T., Horisawa, T., Tokuda, K., Ishiyama, T., Ogasa, M., Tagashira, R., Matsumoto, K., Nishikawa, H., Ueda, Y., Toma, S., 2010. Pharmacological profile of lurasidone, a novel antipsychotic agent with potent 5-hydroxytryptamine 7 (5-HT7) and 5-HT1A receptor activity. J. Pharmacol. Exp. Ther. 334 (1), 171-181.
Kim, Y., Bang, H., Kim, D., 2000. TASK-3, a new member of the tandem pore K(+) channel family. J. Biol. Chem. 275 (13), 9340-9347.
Kim, Y., Giusti-Rodriguez, P., Crowley, J. J., Bryois, J., Nonneman, R. J., Ryan, A. K., Quackenbush, C. R., Iglesias-Ussel, M. D., Lee, P. H., Sun, W., de Villena, F. P., Sullivan, P. F., 2018. Comparative genomic evidence for the involvement of schizophrenia risk genes in antipsychotic effects. Mol. Psychiatry 23 (3), 708-712.
Kumar, R., Corbett, M A., Smith, N. J., Jolly, L. A., Tan, C., Keating, D. J., Duffield, M. D., Utsumi, T., Moriya, K., Smith, K. R., Hoischen, A., Abbott, K, Harbord, M. G., Compton, A. G., Woenig, J. A., Arts, P., Kwint, M., Wieskamp, N., Gijsen, S., Veltman, J. A., Bahlo, M., Gleeson, J. G., Haan, E., Gecz, J., 2015. Homozygous mutation of STXBP5L explains an autosomal recessive infantile-onset neurodegenerative disorder. Hum. Mol. Genet. 24 (7), 2000-2010.
Lavedan, C., Licamele, L., Volpi, S., Hamilton, J., Heaton, C., Mack, K., Lannan, R., Thompson, A., Wolfgang, C. D., Polymeropoulos, M. H., 2009. Association of the NPAS3 gene and five other loci with response to the antipsychotic iloperidone identified in a whole genome association study. Mol. Psychiatry 14 (8), 804-819.
Le Clerc, S., Taing, L., Fond, G., Meary, A., Llorca, P. M., Blanc, O., Beaune, P., Rajagopal, K., Jamain, S., Tamouza, R., Zagury, J. F., Leboyer, M., 2015. A double amino-acid change in the HLA-A peptide-binding groove is associated with response to psychotropic treatment in patients with schizophrenia. Transl. Psychiatry 5, e608.
de Leeuw, C A., Mooij, J. M., Heskes, T., Posthuma, D., 2015. MAGMA: generalized gene-set analysis of GWAS data. PLoS Comput. Biol. 11 (4), e1004219.
Li, J., Meltzer, H. Y., 2014. A genetic locus in 7p12. 2 associated with treatment resistant schizophrenia. Schizophr. Res. 159 (2), 333-339.
Li, Q., Wineinger, N. E., Fu, D. J., Libiger, O., Alphs, L., Savitz, A., Gopal, S., Cohen, N., Schork, N. J., 2017. Genome-wide association study of paliperidone efficacy. Pharmacogenet. Genomics 27 (1), 7-18.
Li, J., Yoshikawa, A., Brennan, M. D., Ramsey, T. L., Meltzer, H. Y., 2018. Genetic predictors of antipsychotic response to lurasidone identified in a genome wide association study and by schizophrenia risk genes. Schizophr. Res. 192, 194-204.
Linden, A. M., Aller, M. I., Leppa, E., Rosenberg, P. H., Wisden, W., Korpi, E. R., 2008. K+channel TASK-1 knockoutmice show enhanced sensitivities to ataxic and hypnotic effects of GABA(A) receptor ligands. J. Pharmacol. Exp. Ther. 327 (1), 277-286.
Loebel, A., Cucchiaro, J., Sarma, K., Xu, L., Hsu, C., Kalali, A. H., Pikalov, A., Potkin, S. G., 2013a. Efficacy and safety of lurasidone 80 mg/day and 160 mg/day in the treatment of schizophrenia: a randomized, double-blind, placebo- and active-controlled trial. Schizophr. Res. 145 (1), 101-109.
Loebel, A., Cucchiaro, J., Xu, J., Sarma, K., Pikalov, A., Kane, J. M., 2013b. Effectiveness of lurasidone vs. quetiapine XR for relapse prevention in schizophrenia: a 12-month, double-blind, noninferiority study. Schizophr. Res. 147 (1), 95-102.
McClay, J. L., Adkins, D. E., Aberg, K., Bukszar, J., Khachane, A. N., Keefe, R. S., Perkins, D. O., McEvoy, J. P., Stroup, T. S., Vann, R. E., Beardsley, P. M., Lieberman, J. A., Sullivan, P. F., van den Oord, E. J., 2011a. Genome-wide pharmacogenomic study of neurocognition as an indicator of antipsychotic treatment response in schizophrenia. Neuropsychopharmacology: Official Publication of the American College of Neuropsychopharmacology 36 (3), 616-626.
McClay, J. L., Adkins, D. E., Aberg, K., Stroup, S., Perkins, D. O., Vladimirov, V. I., Lieberman, J. A., Sullivan, P. F., van den Oord, E. J., 2011b. Genome-wide pharmacogenomic analysis of response to treatment with antipsychotics. Mol. Psychiatry 16 (1), 76-85.
Meltzer, H. Y., 2012. Serotonergic mechanisms as targets for existing and novel antipsychotics. Current Antipsychotics. Springer, pp. 87-124.
Meltzer, H. Y., Cucchiaro, J., Silva, R., Ogasa, M., Phillips, D., Xu, J., Kalali, A. H., Schweizer, E., Pikalov, A., Loebel, A., 2011a. Lurasidone in the treatment of schizophrenia: a randomized, double-blind, placebo- and olanzapine-controlled study. Am. J. Psychiatr. 168 (9), 957-967.
Meltzer, H. Y., Horiguchi, M., Massey, B. W., 2011b. The role of serotonin in the NMDA receptor antagonist models of psychosis and cognitive impairment. Psychopharmacology 213 (2-3), 289-305.
Mexal, S., Frank, M., Berger, R., Adams, C. E., Ross, R. G., Freedman, R., Leonard, S., 2005. Differential modulation of gene expression in the NMDA postsynaptic density of schizophrenic and control smokers. Brain Res. Mol. Brain Res. 139 (2), 317-332.
Meyer, J. M., Loebel, A. D., Schweizer, E., 2009. Lurasidone: a new drug in development for schizophrenia. Expert Opin. Investig. Drugs 18 (11), 1715-1726.
Mishra, A., Macgregor, S., 2015. VEGAS2: software for more flexible gene-based testing. Twin Research and Human Genetics: The Official Journal of the International Society for Twin Studies 18 (1), 86-91.
Nasrallah, H. A., Silva, R., Phillips, D., Cucchiaro, J., Hsu, J., Xu, J., Loebel, A., 2013. Lurasidone for the treatment of acutely psychotic patients with schizophrenia: a 6-week, randomized, placebo-controlled study. J. Psychiatr. Res. 47 (5), 670-677.
Need, A. C., Keefe, R. S., Ge, D., Grossman, I., Dickson, S., McEvoy, J. P., Goldstein, D. B., 2009. Pharmacogenetics of antipsychotic response in the CATIE trial: a candidate gene analysis. Eur. J. Hum. Genet. 17 (7), 946-957.
Nepusz, T., Yu, H., Paccanaro, A., 2012. Detecting overlapping protein complexes in protein-protein interaction networks. Nat. Methods 9 (5), 471-472.
Page, S. C., Hamersky, G. R., Gallo, R. A., Rannals, M. D., Calcaterra, N. E., Campbell, M. N., Mayfield, B., Briley, A., Phan, B. N., Jaffe, A. E., Maher, B. J., 2018. The schizophrenia and autism-associated gene, transcription factor 4 regulates the columnar distribution of layer 2/3 prefrontal pyramidal neurons in an activity-dependent manner. Mol. Psychiatry 23 (2), 304-315.
Pang, D. S., Robledo, C. J., Can, D. R., Gent, T. C., Vyssotski, A. L., Caley, A., Zecharia, A. Y., Wisden, W., Brickley, S. G., Franks, N. P., 2009. An unexpected role for TASK-3 potassium channels in network oscillations with implications for sleep mechanisms and anesthetic action. Proc. Natl. Acad. Sci. U.S.A 106 (41), 17546-17551.
Park, C., Falls, W., Finger, J. H., Longo-Guess, C. M., Ackerman, S. L., 2002. Deletion in Catna2, encoding alpha N-catenin, causes cerebellar and hippocampal lamination defects and impaired startle modulation. Nat. Genet. 31 (3), 279-284.
Purcell, S. M., Moran, J. L., Fromer, M., Ruderfer, D., Solovieff, N., Roussos, P., O'Dushlaine, C., Chambert, K., Bergen, S. E., Kahler, A., Duncan, L., Stahl, E., Genovese, G., Fernandez, E., Collins, M. O., Komiyama, N. H., Choudhary, J. S., Magnusson, P. K, Banks, E., Shakir, K., Garimella, K., Fennell, T., DePristo, M., Grant, S. G., Haggarty, S. J., Gabriel, S., Scolnick, E. M., Lander, E. S., Hultman, C. M., Sullivan, P. F., McCarroll, S. A., Sklar, P., 2014. A polygenic burden of rare disruptive mutations in schizophrenia. Nature 506 (7487), 185-190.
Ripke, S., Neale, B. M., Corvin, A., Schizophrenia Working Group of the Psychiatric Genomics Consortium, 2014. Biological insights from 108 schizophrenia-associated genetic loci. Nature 511 (7510), 421-427.
Ruderfer, D. M., Charney, A. W., Readhead, B., Kidd, B. A., Kahler, A. K., Kenny, P. J., Keiser, M. J., Moran, J. L., Hultman, C. M., Scott, S. A., Sullivan, P. F., Purcell, S. M., Dudley, J. T., Sklar, P., 2016. Polygenic overlap between schizophrenia risk and antipsychotic response: a genomic medicine approach. The Lancet. Psychiatry 3 (4), 350-357.
Sacchetti, E., Magri, C., Minelli, A., Valsecchi, P., Traversa, M., Calza, S., Vita, A., Gennarelli, M., 2017. The GRM7 gene, early response to risperidone, and schizophrenia: a genome-wide association study and a confirmatory pharmacogenetic analysis. Pharmacogenomics J. 17 (2), 146-154.
Sanford, M., 2013. Lurasidone. CNS Drugs 27 (1), 67-80. Smith, A., Bourdeau, L, Wang, J., Bondy, C. A., 2005. Expression of Catenin family members CTNNA1, CTNNA2, CTNNB1 and RIP in the primate prefrontal cortex and hippocampus. Brain Res. Mol. Brain Res. 135 (1-2), 225-231.
Stevenson, J. M., Reilly, J. L., Harris, M. S., Patel, S. R., Weiden, P. J., Prasad, K. M., Badner, J. A., Nimgaonkar, V. L., Keshavan, M. S., Sweeney, J. A., Bishop, J. R., 2016. Antipsychotic pharmacogenomics in first episode psychosis: a role for glutamate genes. Transl. Psychiatry 6, e739.
Sullivan, P., Lin, D., Tzeng, J., van den Oord, E., Perkins, D., Stroup, T., Wagner, M., Lee, S., Wright, F., Zou, F., 2009. Genomewide association for schizophrenia in the CATIE study: results of stage 1. Mol. Psychiatry 14 (12) (1144-1144).
Terracciano, A., Esko, T., Sutin, A. R., de Moor, M. H., Meirelles, O., Zhu, G., Tanaka, T., Giegling, I., Nutile, T., Realo, A., Allik, J., Hansell, N. K., Wright, M. J., Montgomery, G. W., Willemsen, G., Hottenga, J. J., Friedl, M., Ruggiero, D., Sorice, R., Sanna, S., Cannas, A., Raikkonen, K., Widen, E., Palotie, A., Eriksson, J. G., Cucca, F., Krueger, R. F., Lahti, J., Luciano, M., Smoller, J. W., van Duijn, C. M., Abecasis, G. R., Boomsma, D. I., Ciullo, M., Costa Jr., P. T., Ferrucci, L., Martin, N. G., Metspalu, A., Rujescu, D., Schlessinger, D., Uda, M., 2011. Meta-analysis of genome-wide association studies identifies common variants in CTNNA2 associated with excitement-seeking. Transl. Psychiatry 1, e49.
Weyn-Vanhentenryck, S. M., Mele, A., Yan, Q., Sun, S., Farny, N., Zhang, Z., Xue, C., Herre, M., Silver, P. A., Zhang, M. Q., Krainer, A R, Darnell, R. B., Zhang, C., 2014. HITS-CLIP and integrative modeling define the Rbfox splicing-regulatory network linked to brain development and autism. Cell Rep. 6 (6), 1139-1152.
Willer, C. J., Li, Y., Abecasis, G. R., 2010. METAL: fast and efficient meta-analysis of genomewide association scans. Bioinformatics 26 (17), 2190-2191.
Wimberley, T., Gasse, C., Meier, S. M., Agerbo, E., MacCabe, J. H., Horsdal, H. T., 2017. Polygenic risk score for schizophrenia and treatment-resistant schizophrenia. Schizophr. Bull. 43 (5), 1064-1069.
Zai, C. C., Tiwari, A. K., Chowdhury, N. I., Yilmaz, Z., de Luca, V., Muller, D. J., Potkin, S. G., Lieberman, J. A., Meltzer, H. Y., Voineskos, A. N., Remington, G., Kennedy, J. L., 2017. Genetic study of neuregulin 1 and receptor tyrosine-protein kinase erbB-4 in tardive dyskinesia. The World Journal of Biological Psychiatry: The Official Journal of the World Federation of Societies of Biological Psychiatry 1-5.
Zhang, Q., Yu, Y., Huang, X. R, 2016. Olanzapine prevents the PCP-induced reduction in the neurite outgrowth of prefrontal cortical neurons via NRG1. Sci. Rep. 6, 19581.
Zhu, Q., Wong, A. K., Krishnan, A., Aure, M. R., Tadych, A., Zhang, Corney, D. C., Greene, C. S., Bongo, L. A., Kristensen, V. N., Charikar, M., Li, K., Troyanskaya, O. G., 2015. Targeted exploration and analysis of large cross-platform human transcriptomic compendia. Nat. Methods 12 (3), 211-214 (213 p following 214).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method comprising:
(a) detecting a polymorphic allele in a sample from a subject and/or receiving results of a test that detects a polymorphic allele in a sample from a subject, wherein the polymorphic allele is associated with a polymorphism in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions; and
(b) administering an atypical antipsychotic drug to the subject after detecting the polymorphic allele and/or after receiving the results of the test.

2. The method of claim 1, wherein the gene is selected from a group consisting of RBFOX1 (A2BP1), PTPRD, LRRC4C, NRXN1, ILIRAPL1, SLITRK1, NTRK3, MAGI1, MAGI2, NBEA, NRG1/3, PCDH7, FGF9, DNAJA3, AP2B1, GRID1, DLX2, FBXO32, CAMATA1, STXBP5L, KALRN, KCNK9, and CTNNA2.

3. The method of claim 1, wherein the subject has a polymorphic allele of a polymorphism associated with RBFOX1 (A2BP1), optionally wherein the polymorphism is selected from rs17674225 (e.g., where the allele is G/T), rs8057315 (e.g., where the allele is C/A/G/T), rs726476 (e.g., where the allele is G/A/C/T), rs8045750 (e.g., where the allele is G/A), rs9924951 (e.g., where the allele is G/A), rs10468333 (e.g., where the allele is C/G/T), rs9933246 (e.g., where the allele is G/C/T), rs8048158 (e.g., where the allele is C/G), rs11077179 (e.g., where the allele is T/C), rs9936248 (e.g., where the allele is C/A), rs11641748 (e.g., where the allele is G/A), rs10459843 (e.g., where the allele is G/A/C), rs9935875 (e.g., where the allele is G/A/C), rs9935962 (e.g., where the allele is C/A), rs11649628 (e.g., where the allele is C/T), rs28405182 (e.g., where the allele is C/A/G/T), rs8048519 (e.g., where the allele is A/G), rs2159535 (e.g., where the allele is G/C), rs11077183 (e.g., where the allele is C/A), rs11077184 (e.g., where the allele is A/C/G), rs7198769 (e.g., where the allele is G/A/T), rs4786173 (e.g., where the allele is G/A), rs4141146 (e.g., where the allele is G/A), rs9935875 (e.g., where the allele is G/A), rs9935962 (e.g., where the allele is C/A), rs8057315 (e.g., where the allele is C/A/G/T), rs8045750 (e.g., where the allele is A/G), rs17674225 (e.g., where the allele is C/G/T), rs12447542 (e.g., where the allele is A/G), rs10500355 (e.g., where the allele is A/T), rs1057521725 (e.g., where the allele is A/G), rs1064794750 (e.g., where the allele is G/C), rs11643447 (e.g., where the allele is A/T), rs11645781 (e.g., where the allele is A/G), rs11866781 (e.g., where the allele is C/T), rs12444931 (e.g., where the allele is A/G), rs12446308 (e.g., where the allele is A/G), rs12921846 (e.g., where the allele is A/T), rs12926282 (e.g., where the allele is A/C), rs1478693 (e.g., where the allele is A//C), rs17139207 (e.g., where the allele is A/G), rs17139244 (e.g., where the allele is A/G), rs17648524 (e.g., where the allele is C/G), rs1906060 (e.g., where the allele is C/T), rs3785234 (e.g., where the allele is C/T), rs4124065 (e.g., where the allele is G/T), rs4146812 (e.g., where the allele is C/T), rs4786816 (e.g., where the allele is A/G), rs4787008 (e.g., where the allele is A/G), rs6500742 (e.g., where the allele is C/T), rs6500744 (e.g., where the allele is C/T), rs6500818 (e.g., where the allele is C/T), rs6500882 (e.g., where the allele is G/T), rs6500963 (e.g., where the allele is C/T), rs716508 (e.g., where the allele is C/T), rs7191721 (e.g., where the allele is A/G), rs7403856 (e.g., where the allele is A/G), rs7498702 (e.g., where the allele is C/T), rs870288 (e.g., where the allele is A/G), rs889699 (e.g., where the allele is A/G), rs9302841 (e.g., where the allele is A/T), rs9924951 (e.g., where the allele is A/G), rs1478697 (e.g., where the allele is A/G/T), and combinations thereof.

3. The method of claim 1, wherein: (i) detecting and/or the test comprises amplifying at least a portion of the gene from the nucleic acid sample and detecting the polymorphism in the amplified portion; (ii) detecting and/or the test comprises sequencing at least a portion of the gene from the nucleic acid sample or from an amplicon obtained by amplifying at least a portion of the gene from the nucleic acid sample; and/or (iii) detecting and/or the test comprises contacting nucleic acid comprising the polymorphism with a nucleic acid probe that hybridizes specifically to nucleic acid comprising the polymorphism.

4. The method of claim 1, wherein detecting and/or the test comprises determining whether the nucleic acid sample is homozygous for the polymorphic allele.

5. The method of claim 1, wherein detecting and/or the test comprises determining whether the nucleic acid sample is heterozygous for the polymorphic allele.

6. The method of claim 1, wherein the nucleic acid sample is obtained from blood or a blood product.

7. The method of claim 1, wherein the subject has a psychiatric disease or disorder selected from the group consisting of schizophrenia, bipolar disorder, and psychiatric depression.

8. The method of claim 1, wherein the subject has schizophrenia and is exhibited symptoms selected from the group consisting of positive symptoms, negative symptoms, cognitive symptoms, and any combination thereof.

9. The method of claim 1, wherein the APD is an atypical APD.

10. The method of claim 1, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

11. The method of claim 1, wherein the APD is an agonist or partial agonist for the 5-HT_{1A} receptor.

12. The method of claim 1, wherein the APD has negligible or no biological activity as a ligand for the H₁ receptor and/or mACh receptor (e.g., where the Kᵢ is >about 5 μM, 10 μM, 50 μM, 100 μM, or 500 μM).

13. The method of claim 1, wherein the atypical APD comprises lurasidone, ziprasidone, clozapine, olanzapine, risperidone, perphenazine, or serindole.

14. A kit or combination comprising:
(a) a nucleic acid reagent that hybridizes specifically to a polymorphic allele of a polymorphism in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions; and
(b) an antipsychotic drug (APD).

15. A method comprising administering an antipsychotic drug (APD) to a subject having a psychiatric disease or disorder after the subject has been determined to have a polymorphic allele in a gene encoding a protein associated with synaptogenic adhesion, scaffolding, neuron-specific splicing regulation, potassium channels which form leak conductances that regulate neuronal excitability, synaptic spine turnover and stability of synaptic contacts, and/or vesicle trafficking and exocytosis in presynaptic neurons and neuromuscular junctions.

16. The method of claim 15, wherein the subject has a polymorphic allele of a polymorphism associated with RBFOX1 (A2BP1), optionally wherein the polymorphism is selected from rs17674225 (e.g., where the allele is G/T), rs8057315 (e.g., where the allele is C/A/G/T), rs726476 (e.g., where the allele is G/A/C/T), rs8045750 (e.g., where the allele is G/A), rs9924951 (e.g., where the allele is G/A), rs10468333 (e.g., where the allele is C/G/T), rs9933246 (e.g., where the allele is G/C/T), rs8048158 (e.g., where the allele is C/G), rs11077179 (e.g., where the allele is T/C), rs9936248 (e.g., where the allele is C/A), rs11641748 (e.g., where the allele is G/A), rs10459843 (e.g., where the allele is G/A/C), rs9935875 (e.g., where the allele is G/A/C), rs9935962 (e.g., where the allele is C/A), rs11649628 (e.g., where the allele is C/T), rs28405182 (e.g., where the allele is C/A/G/T), rs8048519 (e.g., where the allele is A/G), rs2159535 (e.g., where the allele is G/C), rs11077183 (e.g., where the allele is C/A), rs11077184 (e.g., where the allele is A/C/G), rs7198769 (e.g., where the allele is G/A/T), rs4786173 (e.g., where the allele is G/A), rs4141146 (e.g., where the allele is G/A), rs9935875 (e.g., where the allele is G/A), rs9935962 (e.g., where the allele is C/A), rs8057315 (e.g., where the allele is C/A/G/T), rs8045750 (e.g., where the allele is A/G), rs17674225 (e.g., where the allele is C/G/T), rs12447542 (e.g., where the allele is A/G), rs10500355 (e.g., where the allele is A/T), rs1057521725 (e.g., where the allele is A/G), rs1064794750 (e.g., where the allele is G/C), rs11643447 (e.g., where the allele is A/T), rs11645781 (e.g., where the allele is A/G), rs11866781 (e.g., where the allele is C/T), rs12444931 (e.g., where the allele is A/G), rs12446308 (e.g., where the allele is A/G), rs12921846 (e.g., where the allele is A/T), rs12926282 (e.g., where the allele is A/C), rs1478693 (e.g., where the allele is A//C), rs17139207 (e.g., where the allele is A/G), rs17139244 (e.g., where the allele is A/G), rs17648524 (e.g., where the allele is C/G), rs1906060 (e.g., where the allele is C/T), rs3785234 (e.g., where the allele is C/T), rs4124065 (e.g., where the allele is G/T), rs4146812 (e.g., where the allele is C/T), rs4786816 (e.g., where the allele is A/G), rs4787008 (e.g., where the allele is A/G), rs6500742 (e.g., where the allele is C/T), rs6500744 (e.g., where the allele is C/T), rs6500818 (e.g., where the allele is C/T), rs6500882 (e.g., where the allele is G/T), rs6500963 (e.g., where the allele is C/T), rs716508 (e.g., where the allele is C/T), rs7191721 (e.g., where the allele is A/G), rs7403856 (e.g., where the allele is A/G), rs7498702 (e.g., where the allele is C/T), rs870288 (e.g., where the allele is A/G), rs889699 (e.g., where the allele is A/G), rs9302841 (e.g., where the allele is A/T), rs9924951 (e.g., where the allele is A/G), rs1478697 (e.g., where the allele is A/G/T), and combinations thereof.

17. The method of claim 15, wherein the APD is an antagonist for one or more of the following sites: α₁-adrenergic receptor, α_{2A}-adrenergic receptor, α_{2C}-adrenergic receptor, D₁ receptor, D₂ receptor, 5-HT_{2A} receptor, 5-HT_{2C} receptor, and 5-HT₇ receptor.

18. The method of claim 15, wherein the APD is an agonist or partial agonist for the 5-HT_{1A} receptor.

19. The method of claim 15, wherein the APD has negligible or no biological activity as a ligand for the H₁ receptor and/or mACh receptor (e.g., where the Kᵢ is >about 5 μM, 10 μM, 50 μM, 100 μM, or 500 μM).

20. The method of claim 15, wherein the atypical APD comprises lurasidone, ziprasidone, clozapine, olanzapine, risperidone, perphenazine, or serindole.
